(19) United States
(12) Patent Application Publication
(10) Pub. No.: US 2024/0385182 A1
(43) Pub. Date: Nov. 21, 2024

(54) MASKED FLUOROGENIC LIPID COMPOUNDS, LIPID PARTICLES COMPRISING THE SAME, AND METHODS OF USE THEREOF

(71) Applicant: Rutgers, The State University of New Jersey, New Brunswick, NJ (US)

(72) Inventors: Enver Cagri Izgu, Piscataway, NJ (US); Bryan Gutierrez, Piscataway, NJ (US); Huseyin Erguven, Piscataway, NJ (US); Liming Wang, Piscataway, NJ (US)

(21) Appl. No.: 18/665,906

(22) Filed: May 16, 2024

Related U.S. Application Data
(60) Provisional application No. 63/466,946, filed on May 16, 2023.

(51) Int. Cl. G01N 33/53; C07F 9/6558; C07F 9/6561; C07F 9/6596; G01N 33/543

(52) U.S. Cl. CPC G01N 33/5308; C07F 9/65583; C07F 9/65586; C07F 9/6561; C07F 9/6596; G01N 33/5432; G01N 2800/7004; G01N 2800/7009

(57) ABSTRACT

The present disclosure relates, in part, to masked fluorogenic lipid compounds, lipid particles (e.g., giant vesicles and/or lipid nanoparticles) comprising the same, and methods of use thereof for the detection of redox agents in a sample. In certain embodiments, the present disclosure provides a method of diagnosing a disease or disorder associated with oxidative, nitrosative, or nitrative stress.

### CROSS-REFERENCE TO RELATED APPLICATIONS

This application claims priority under 35 U.S.C. § 119(e) to U.S. Provisional Patent Application No. 63/466,946, filed May 16, 2023, which is incorporated herein by reference in its entirety.

### STATEMENT REGARDING FEDERALLY SPONSORED RESEARCH OR DEVELOPMENT

This invention was made with government support under EB029548 awarded by the National Institutes of Health. The government has certain rights in the invention.

### BACKGROUND

Lipid membranes have diverse biological functions and are among the most essential components of eukaryotic cells. Uncontrolled redox physiochemistry impacts the integrity and dynamics of these key components, and consequences of redox dysfunction are associated with critical human diseases.

There is thus a need in the art for compounds and compositions useful as bioimaging tools to enable real-time, subcellular investigation of lipid membranes and methods of use thereof. The present disclosure addresses this need.

### BRIEF SUMMARY

In one aspect, the disclosure provides a compound of formula (I), or a salt, solvate, stereoisomer, or isotopologue thereof, wherein R^{1a}, R^{1b}, R^{1c}, R^{2a}, R^{2b}, R³, X^{1a}, X^{1b}, X², m, n, and o are defined elsewhere herein:

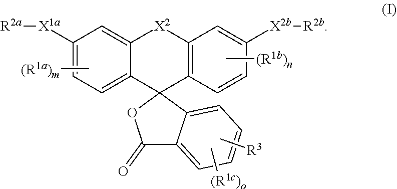

In another aspect, the disclosure provides a compound of formula (II), or a salt, solvate, stereoisomer, or isotopologue thereof, wherein R^{1a}, R^{1b}, R^{2a}, R³, X^{1a}, and m are defined elsewhere herein:

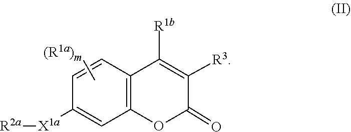

In another aspect, the disclosure provides a compound of formula (III), or a salt, solvate, stereoisomer, or isotopologue thereof, wherein R^{1a}, R⁴, R⁷, L¹, and p are defined elsewhere herein:

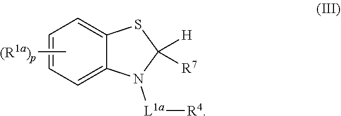

In another aspect, the disclosure provides a compound of formula (IV), or a salt, solvate, stereoisomer, or isotopologue thereof, wherein R^{1a}, R^{1b}, R^{2a}, R³, X^{1a}, and m are defined elsewhere herein:

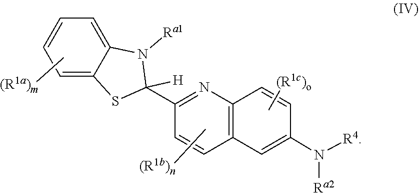

In another aspect, the disclosure provides a lipid particle. In certain embodiments, the lipid particle comprises at least one compound of any one of formulae (I)—(IV) and at least one additional lipid.

In another aspect, the disclosure provides a method of detecting a redox biomarker in a sample. In certain embodiments, the method comprises contacting the compound of any of Formulae (I)—(IV) of the disclosure and/or a lipid particle of the disclosure with the sample to provide a mixture. In certain embodiments, the method further comprises detecting a fluorescent signal in the mixture, wherein the fluorescent signal is indicative of a redox biomarker in the sample.

In another aspect, the disclosure provides a method of diagnosing a disease or disorder associated with oxidative, nitrosative, or nitrative stress in a subject. In certain embodiments, the method comprises contacting the compound of any of Formulae (I)—(IV) and/or a lipid particle of the disclosure with a sample obtained from the subject to provide a mixture. In certain embodiments, the method further comprises detecting a fluorescent signal in the mixture, wherein the fluorescent signal is indicative of a disease or disorder associated with oxidative, nitrosative, or nitrative stress.

### BRIEF DESCRIPTION OF THE FIGURES

The drawings illustrate generally, by way of example, but not by way of limitation, various embodiments of the present application.

FIG. 1: overview of redox sensors built from biomimetic lipid membranes. Each membrane system contains a mixture of natural lipids (e.g., mono-unsaturated phospholipids and cholesterol) and a synthetic lipid that specifically reacts with a target redox-active species (e.g., H₂S, H₂O₂, or ONOO⁻, inter alia).

FIG. 2A: chemical synthesis and fluorescence activation mechanism of TEG-TC-ONOO⁻ and DPPC-TC-ONOO⁻; THPTA (i.e., tris(3-hydroxypropyltriazolyl-methyl)amine). FIG. 2B: depicts peroxynitrite-mediated fluorescence generation from TEG-TC-ONOO⁻ and DPPC-TC-ONOO⁻.

FIG. 3: non-limiting syntheses of exemplary phospholipid-based H₂S probe DPPC-TC-H₂S and exemplary H₂O₂ probe DPPC-TC-H₂O₂ and non-amphiphilic analogues thereof (i.e., TEG-TC-H₂S and TEG-TC-H₂O₂, respectively).

FIG. 4: non-limiting proposed syntheses of a non-limiting exemplary redox probe for sensing H₂O₂.

FIG. 5: schemes depicting non-limiting reactions between non-limiting redox-sensing probes and their cognate redox species.

FIGS. 6A-6D: non-limiting proposed syntheses of non-limiting redox probes that sense ONOO⁻ (FIG. 6A), NO· (FIG. 6B), O₂·⁻ (FIG. 6C), and H₂S (FIG. 6D).

FIG. 7A: time-dependent change in relative fluorescence intensities for probe TEG-TC-ONOO⁻ (1 μM) and compound TEG-TC (1 μM) in Tris (50 mM, pH 7.5) upon addition of ONOO⁻ (600 μM). Filled square: TEG-TC, hollow square: TEG-TC+ONOO⁻, filled circle: TEG-TC-ONOO⁻, and hollow circle: TEG-TC-ONOO⁻+ONOO⁻. Error bars represent standard deviation, n=3. FIG. 7B: relative fluorescence intensity (F) with respect to the background fluorescence intensity (F0) of TEG-TC-ONOO⁻ in the absence or presence of redox species. Probe TEG-TC-ONOO⁻ (1 μM) was dissolved in Tris buffer (50 mM, pH 7.5) and subjected to RNS and ROS (600 μM): ONOO⁻, NO·, H₂O₂, HO·, ¹O₂, and O·⁻. Data represent FIFO measurements for samples incubated for 60 min. Plate reader was set to excitation at 405 nm and emission at 475 nm. Error bars represent standard deviation, n=3.

FIG. 8A: conceptual illustration of function of the model protocell. FIG. 8B: confocal images acquired for untreated (top panel) and peroxynitrite-treated (middle and bottom panels) GVs. FIG. 8C: confocal images acquired for GVs treated with other RNS and ROS. FIGS. 8B-8C: vesicles were prepared from POPC (98.5%, molar ratio) and DPPC-TC-ONOO⁻ (1%) via electroformation. Liss-Rhod PE dye (red, 560 nm excitation) was used at 0.5 mol % to label vesicle membranes. In the absence of peroxynitrite or in the presence of other RNS or ROS (100 μM), vesicles emit minimal fluorescence at DPPC-TC channel (405/475 nm). GVs are activated to emit strong fluorescence when peroxynitrite (100 μM) is present. Fluorescence images are shown for each field of view to demonstrate the localization of the fluorescent phospholipid DPPC-TC, generated by peroxynitrite in situ, with respect to the vesicle membranes. Scale bar=5 μm.

FIGS. 9A-9E: confocal imaging of lipid environments targeted by ONOO⁻ in live HeLa cells. Cells were seeded into glass microscope dishes, incubated with LNPs (50 μM) and stained with organelle trackers and/or actin dye prior to imaging. LNPs obtained from a 47.5:47.5:5.0 molar ratio of DOTMA, DOPE, and DPPC-TC-ONOO⁻. FIG. 9A: cells treated with LNPs, stimulated with IFN-γ/LPS/PMA. Time coarse images acquired upon PMA treatment. FIG. 9B: cells treated with LNPs and 1400 W. FIG. 9C: cells treated with LNPs, then with 1400 W and IFN-γ/LPS/PMA. FIGS. 9D-9E: quantitative colocalization study of cells treated with LNPs and stimulated with IFN-γ/LPS/PMA. Pearson's correlation coefficient (PCC) was calculated for quantitative assessment. Organelle trackers: ER-Tracker™, MitoTracker™, CellLight™ Golgi-RFP, LysoTracker™. Cell cytoskeleton was assessed with CellMask™ Deep Red Actin Tracking Stain. DPPC-TC channel: 405/475 nm. Scale bars=20 μm (FIGS. 9A-9D) or 5 μm (FIG. 9E).

FIGS. 10A-10E: confocal imaging of lipid environments targeted by ONOO⁻ in live RAW246.7 cells. Cells were seeded into glass microscope dishes, incubated with LNPs (50 μM) and stained with organelle trackers and/or actin dye prior to imaging. LNPs obtained from a 47.5:47.5:5.0 molar ratio of DOTMA, DOPE, and DPPC-TC-ONOO⁻. FIG. 10A: cells treated with LNPs, stimulated with LPS. Time coarse images acquired after addition of LPS. FIG. 10B: cells treated with LNPs and 1400 W. FIG. 10C: cells treated with LNPs, then with 1400 W and LPS. FIGS. 10D-10E: quantitative colocalization study of cells treated with LNPs and stimulated with LPS. Pearson's correlation coefficient (PCC) was calculated for quantitative assessment of colocalization using organelle trackers as described above. Scale bars=20 μm (FIGS. 10A-10D) or 5 μm (FIG. 10E).

FIGS. 11A-11B: flow cytometry analysis of myeloid-derived BAL cells for migratory phenotype and DPPC-TC-ONOO⁻ probe activity in murine model of ALI. FIG. 11A: conceptual illustration of ITB-induced ALI and LNP instillation. FIG. 11B: classification of myeloid-derived BAL cells by migratory phenotype (CD11b+) and DPPC-TC fluorescence. Quadrant plots are shown for individual mice, which were used to quantify DPPC-TC as a measure of peroxynitrite generation.

FIGS. 12A-12B: HRMS analyses of reaction mixtures comprising TEG-TC-H₂S and H₂S (FIG. 12A) or TEG-TF-H₂O₂ and H₂O₂(FIG. 12B). The spectral data were collected in positive ionization mode and through direct injection of reaction mixture aliquots taken after either 3 hours (FIG. 12A) or 1 h (FIG. 12B) of gentle mixing. Reaction conditions: TEG-TC-H₂S (20 μM), H₂S (600 μM), Tris (50 mM, pH 7.5) (FIG. 12A); and TEG-TF-H₂O₂ (50 PM), H₂O₂ (100 μM), Tris (50 mM, pH 7.5) (FIG. 12B).

FIGS. 13A-13D: specificity (FIGS. 13A-13B) and limit of detection (LoD) (FIGS. 13C-13D) of TEG-TC-H₂S and TEG-TF-H₂O₂. LoD measurements for the probes TEG-TC-H₂S (390/475 nm) and TEG-TF-H₂O₂ (485/525 nm) were performed in Tris buffer (50 mM, pH 7.5). Error bars represent the standard deviation; n=3. Single-tailed Student's t test: *, P<0.05; **, P<0.01; ***, P<0.001; ****, P<0.0001.

FIGS. 14A-14B: giant vesicles (GVs) that can sense reductive (FIG. 14A) or oxidative (FIG. 14B) environments. Confocal images acquired for H₂S-sensing GV at 405/475 nm (ex/em) (FIG. 14A) and H₂O₂ sensing GV at 490/520 nm (ex/em) (FIG. 14B). Lipid compositions of GVs: POPC, POPG, cholesterol, and DPPC-TC-H₂S (48:21:30:1 molar ratio; FIG. 14A); and POPC, POPG, cholesterol, and DPPC-TF-H₂O₂ (48.5:21:30:0.5; FIG. 14B). To label vesicle membranes, Liss-Rhod PE dye (563/580 nm) was introduced to the lipid mixture at an insignificant molar ratio (0.1 mol %). Concentrations of RSS and ROS were set to 500 and 100 μM, respectively. Scale bar=5 μm.

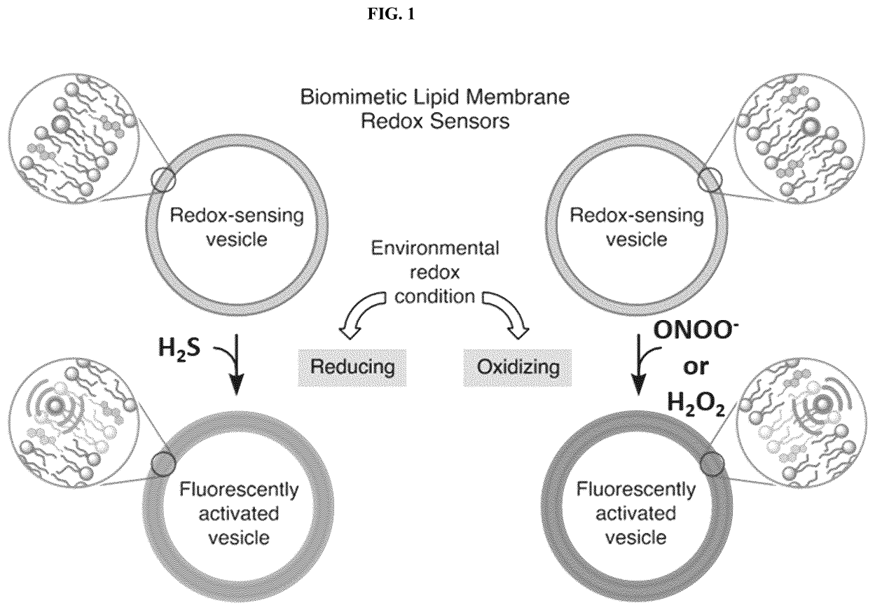

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### DESCRIPTION

In one aspect, the present disclosure relates to lipid compounds and/or lipid mimetic compounds comprising masked fluorophores. In certain embodiments, the masked fluorophores are unmasked to provide fluorogenic species upon chemical modification by a suitable redox agents and/or biomarkers (e.g., nitric oxide, hydrogen peroxide, and hydrogen sulfide, inter alia).

Peroxynitrite (ONOO⁻) Lipid membranes and lipid-rich environments are potential targets of reactive nitrogen and oxygens species (RNS and ROS). A major source of lipid membrane damage is peroxynitrite (ONOO−), which is produced through the reaction between nitric oxide (NO·) and superoxide (O₂·⁻) at near diffusional rates (˜5×10⁹ mol⁻¹·s⁻¹). The majority of ONOO− formation occurs within the lipid portion of the cell, as NO· is over a thousand times more soluble in hydrophobic environments. It has been proposed that over 90% of ONOO− formed within a biological system will occur in the membrane. At physiological pH, peroxynitrite is readily protonated, which has an estimated biological half-life of ˜10-20 ms. As a result, strategies to investigate the reactivity of peroxynitrite in biological systems are generally challenging and indirect.

Without wishing to be limited by any theory, the mechanism of peroxynitrite-mediated lipid damage has been proposed to involve hydrogen-abstraction from unsaturated lipids, such as linolenic acid and arachidonic acid, generating carbon-centered lipid radicals. These radicals react with nearby unsaturated lipids and are trapped by molecular oxygen, forming lipid peroxides. Peroxynitrite reactivity has been linked to protein aggregation in neurons associated with Parkinson's disease and synucleinopathies, while the same reactivities have also been associated with the progression of cardiovascular diseases like diabetic cardiomyopathy.

Inducible nitric oxide synthase (iNOS) catalyzes the production of NO· from L-arginine and molecular oxygen (O₂) in macrophages in response to various stimuli, including bacterial or viral infections, inflammation, and proinflammatory cytokines. In the case of RAW264.7, a widely used macrophage model, peroxynitrite production can be stimulated by lipopolysaccharide (LPS). LPS binds Toll-like receptor 4 (TLR4), which triggers the activation of nuclear factor kappa B (NF-κB) signalling, resulting in the production of iNOS and ROS. In HeLa, peroxynitrite production through iNOS is facilitated by the combination of interferon-γ (IFN-γ), LPS, and phorbol myristate acetate (PMA). The expression and activity of iNOS must be tightly regulated, because excessive or prolonged iNOS activation is detrimental to the host, leading to tissue damage and fibrosis. Macrophage upregulation of iNOS production in response to injury has been a characteristic of pro-inflammatory activation.

One such model is intratracheal bleomycin (ITB)-induced ALI. The extent of pulmonary ITB injury has also been demonstrated to be heavily iNOS dependent. This system thus presents a relevant biological model in which to examine the efficacy of ONOO− detection in vivo.

As peroxynitrite is emerging to be a critical reactive species involved in cellular stress and different forms of pathophysiologies, developing molecular tools to investigate its impact on lipid clusters, membranes, and lipid-rich organelles or tissues has become increasingly important. Although there is a significant and evolving interest in developing biosensing tools to study lipid membranes, technologies for investigating peroxynitrite reactivity in biological lipid membranes remain underdeveloped.

Furthermore, state-of-the-art methods measure redox in vivo through indirect techniques, such as immunohistochemistry, chemiluminesce, or EPR (electron paramagnetic resonance).

In one aspect, the present disclosure relates to the development, characterization, membrane localization, and multifaceted utilization of DPPC-TC-ONOO⁻, a high-fidelity and site specific peroxynitrite probe derived from 1,2-dipalmitoyl-rac-glycero-3-phosphocholine (rac-DPPC). This designer phospholipid senses ONOO⁻ proximal to the lipid membranes in both biomimetic systems and mammalian cells with diverse origins.

Using DPPC-TC-ONOO⁻ and 1-palmitoyl-2-oleoyl-sn-glycero-3-phosphocholine (POPC), synthetic mimics of protocell membranes in the form of giant vesicles (GVs) were prepared. These GVs responded to ONOO⁻ by lighting up at the membrane, with the vesicle structures remaining intact. Additionally, they displayed excellent selectivity against other redox species. Validating that the biophysical integrity of the membranes is retained before and after peroxynitrite sensing, DPPC-TC-ONOO⁻ was then utilized in live HeLa and RAW264.7 cells. Real-time confocal imaging demonstrated substantial fluorescence enhancement of the ER in HeLa under nitrative stress induced through endogenous stimulation with IFN-γ/LPS/PMA.

Assays for RAW264.7 cells used LPS-mediated stimulation, which led to enhanced fluorescence of lipid clusters in both the ER and Golgi. The redox selectivity of DPPC-TC-ONOO⁻ was confirmed to be iNOS-dependent via control experiments that use an inhibitor of iNOS. The biological potential of DPPC-TC-ONOO⁻ was further demonstrated through a murine model of ALI. Immunostaining of bronchoalveolar lavage (BAL) cells, which normally are 95% or more macrophages, indicated that DPPC-TC-ONOO⁻ responds to iNOS-dependent nitrative stress upon intratracheal bleomycin (ITB) challenge.

Non-Limiting Additional Redox Agents (e.g., H₂O₂ and H₂S)

Redox reactions (i.e., chemical transformations involving a change in atomic oxidation state) form a fundamental basis of physiochemistry in all known life forms. Monitoring redox-active species in proximity of lipid environments can provide insight into the diffusibility of these species across bilayer membranes, a critical physiochemical parameter for intercellular signaling. In addition, molecular probes that sense oxidative or reductive conditions and localize to lipid microenvironments allow for the investigation of the biophysical integrity of lipid membranes exposed to abrupt redox alterations. Hydrogen sulfide (H₂S), a reactive sulfur species or RSS, and hydrogen peroxide (H₂O₂), a reactive oxygen species or ROS, are two redox-active molecules with significant roles in diverse biological processes, ranging from cellular communication and metabolic regulation to pathophysiology. H₂S is a potent, nucleophilic, reducing agent that has cytoprotective properties against oxidative stress and can serve as a gasotransmitter in mammals. H₂O₂ is an aerobic metabolite that serves as an oxidant, primarily generated from superoxide radical (O₂·⁻) by superoxide dismutases. It is involved in a number of biological processes, including signal transduction, cell differentiation and proliferation, immune response, mitochondrial dysfunction, and oncogene activity.

There has been an increasing degree of appreciation for both H₂S and H₂O₂ due to their roles in cellular communication, yet the underlying mechanisms of how they modulate signaling are progressively evolving areas of research. As a gasotransmitter, H₂S is involved in signal transduction pathways pertaining to neurological and cardiovascular processes. Typically, it diffuses through lipid membranes without specialized channels. H₂O₂ transport across membranes occurs through simple diffusion or is facilitated by aquaporins. Outside-the-box chemical technologies could elucidate poorly understood mechanisms by which these transient inorganic molecules affect cell homeostasis, aging, pathology, and cellular signaling.

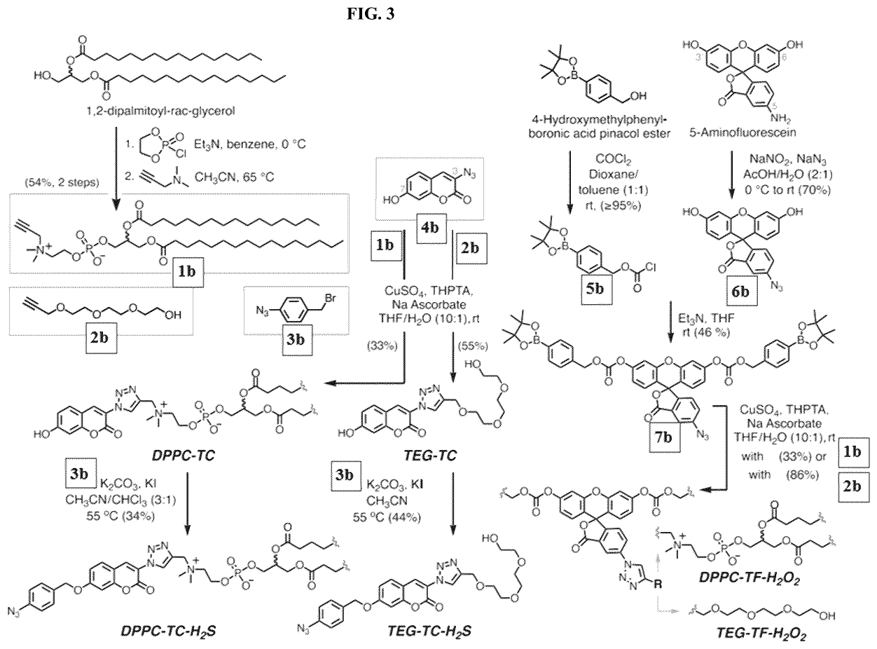

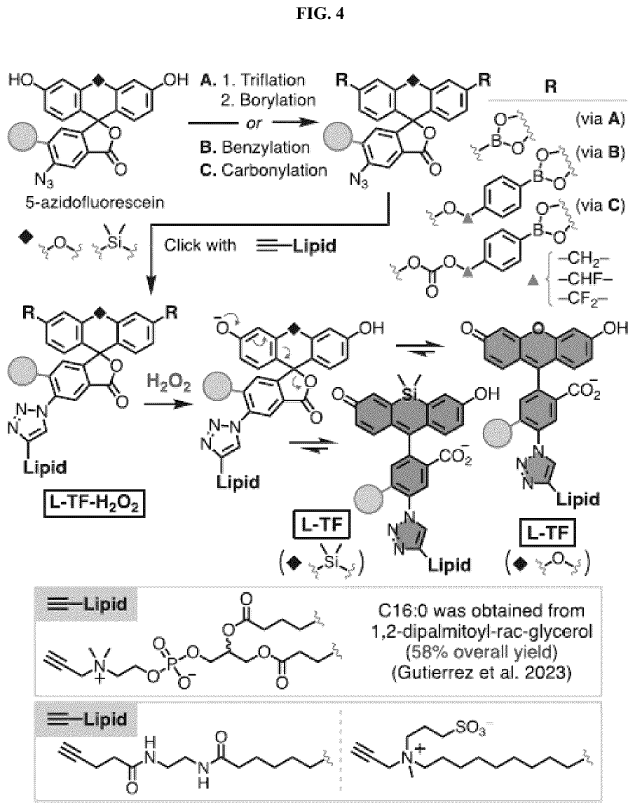

In one aspect, the present disclosure relates to the development of non-limiting, exemplary, designer phospholipids capable of activity-based redox sensing, each possessing a redox-sensitive fluorogenic headgroup and the 1,2-dipalmitoyl-rac-glycero-3-phosphocholine (rac-DPPC) amphiphile: DPPC-TC-H₂S and DPPC-TF-H₂O₂ (FIG. 3).

These designer lipids, along with POPC, 1-palmitoyl-2-oleoyl-sn-glycero-3-(phosphor-rac-(1-glycerol)) (POPG), and cholesterol, were used to construct giant vesicles (GVs) capable of sensing and responding to either H₂S or H₂O₂. When incubated with the target redox species, GVs lit up at the lipid bilayer membrane, with the vesicle structure remaining intact. They displayed good-to-excellent selectivity against other RSS or ROS that represent a large scope of physiologically relevant redox species.

This bottom-up approach enables the proof-of-concept development of lipid self-assemblies capable of sensing redox alterations, offering previously inaccessible functions to biomimetic membrane designs and soft materials.

### Definitions

The term “about” as used herein can allow for a degree of variability in a value or range, for example, within 10%, within 5%, or within 1% of a stated value or of a stated limit of a range, and includes the exact stated value or range.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “alkylene” or “alkylenyl” as used herein refers to a bivalent saturated aliphatic radical (e.g., —CH₂—, —CH₂CH₂—, and —CH₂CH₂CH₂—, inter alia). In certain embodiments, the term may be regarded as a moiety derived from an alkene by opening of the double bond or from an alkane by removal of two hydrogen atoms from the same (e.g., —CH₂—) different (e.g., —CH₂CH₂—) carbon atoms. Similarly, the terms “heteroalkylenyl”, “cycloalkylenyl”, “heterocycloalkylenyl”, and the like, as used herein, refer to a divalent radical of the moiety corresponding to the base group (e.g., heteroalkyl, cycloalkyl, and/or heterocycloalkyl). A divalent radical possesses two open valencies at any position(s) of the group, wherein each radical may be on a carbon atom or heteroatom. Thus, the divalent radical may form a single bond to two distinct atoms or groups, or may form a double bond with one atom.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol, and other anionic modifying groups joined to neutral lipids.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “cationic lipid” refers to any of a number of lipid species that carry a net positive charge at a selected pH, such as physiological pH (e.g., pH of about 7.0). It has been found that cationic lipids comprising alkyl chains with multiple sites of unsaturation, e.g., at least two or three sites of unsaturation, are particularly useful for forming lipid particles with increased membrane fluidity. A number of cationic lipids and related analogs, which are also useful in the present disclosure, have been described in U.S. Patent Publication Nos. 20060083780 and 20060240554; U.S. Pat. Nos. 5,208,036; 5,264,618; 5,279,833; 5,283,185; 5,753,613; and 5,785,992; and PCT Publication No. WO 96/10390, the disclosures of which are herein incorporated by reference in their entirety for all purposes. Non-limiting examples of cationic lipids are described in detail herein. In some cases, the cat-ionic lipids comprise a protonatable tertiary amine (e.g., pH titratable) head group, C₁₈ alkyl chains, ether linkages between the head group and alkyl chains, and 0 to 3 double bonds. Such lipids include, e.g., DSDMA, DLinDMA, DLenDMA, and DODMA.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

The term “giant vesicle” or “unilamellar liposome” as used herein refers to a spherical liposome or vesicle, bounded by a single bilayer of an amphiphilic lipid or a mixture of such lipids, containing aqueous solution inside the chamber, wherein the giant vesicle has a size range of 1-200 μm.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “helper lipid” as used herein refers to a lipid capable of increasing the effectiveness of delivery of lipid-based particles such as cationic lipid-based particles to a target, preferably into a cell. The helper lipid can be neutral, positively charged, or negatively charged. In certain embodiments, the helper lipid is neutral or negatively charged. Non-limiting examples of helper lipids include 1,2-distearoyl-sn-glycero-3-phosphocholine (DSPC), 1,2-di-(9Z-octadecenoyl)-sn-glycero-3-phosphoethanolamine (DOPE), POPC, and 1,2-dioleoyl-sn-glycero-3-phosphocholine (DOPC).

The term “heteroalkyl” as used herein by itself or in combination with another term, means, unless otherwise stated, a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) (e.g., O, N, P, and S) may be placed at any interior position of the heteroalkyl group or at either terminal position at which the group is attached to the remainder of the molecule.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heterocycloalkyl” as used herein refers to an aliphatic, partially unsaturated or fully saturated, 3- to 14-membered ring system, including single rings of 3 to 8 atoms and bi- and tricyclic ring systems where at least one of the carbon atoms of the ring is replaced with a heteroatom such as, but not limited to, nitrogen, oxygen, sulfur, or phosphorus. A heterocycloalkyl can include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited, to the following exemplary groups: pyrrolidinyl, pyrazolinyl, pyrazolidinyl, imidazolinyl, imidazolidinyl, piperidinyl, piperazinyl, azetidinyl, oxazolidinyl, isoxazolidinyl, morpholinyl, thiazolidinyl, isothiazolidinyl, and tetrahydrofuryl.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “ionizable lipid” as used herein refers to a lipid (e.g., a cationic lipid) having at least one protonatable or deprotonatable group, such that the lipid is positively charged at a pH at or below physiological pH (e.g., pH 7.4), and neutral at a second pH, preferably at or above physiological pH. It will be understood by one of ordinary skill in the art that the addition or removal of protons as a function of pH is an equilibrium process, and that the reference to a charged or neutral lipid refers to the nature of the predominant species and does not require that all of the lipid be present in the charged or neutral form. Generally, ionizable lipids have a pKₐ of the protonatable group in the range of about 4 to about 7.

The term “lipid” refers to a group of organic compounds that include, but are not limited to, esters of fatty acids and are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

The term “lipid particle” is used herein to refer to a lipid formulation that can be used to deliver an active agent or therapeutic agent, to a target site of interest.

The term “neutral lipid” refers to any of a number of lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

The term “non-cationic lipid” refers to any amphipathic lipid as well as any other neutral lipid or anionic lipid.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, O-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such compounds are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, PA), which is incorporated herein by reference.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, sulfonate groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

A “therapeutic” treatment is a treatment administered to a subject who exhibits signs of pathology, for the purpose of diminishing or eliminating those signs.

The term “therapeutic protein” as used herein refers to a protein or peptide which has a positive or advantageous effect on a condition or disease state of a subject when provided to the subject in a therapeutically effective amount. In some embodiments, a therapeutic protein or peptide has curative or palliative properties and may be administered to ameliorate, relieve, alleviate, reverse, delay onset of or lessen the severity of one or more symptoms of a disease or disorder. A therapeutic protein or peptide may have prophylactic properties and may be used to delay the onset of a disease or to lessen the severity of such disease or pathological condition. The term “therapeutic protein” includes entire proteins or peptides, and can also refer to therapeutically active fragments thereof. It can also include therapeutically active variants of a protein. Exemplary therapeutic proteins include, but are not limited to, an analgesic protein, an anti-inflammatory protein, an anti-proliferative protein, an proapoptotic protein, an anti-angiogenic protein, a cytotoxic protein, a cytostatic protein, a cytokine, a chemokine, a growth factor, a wound healing protein, a pharmaceutical protein, or a pro-drug activating protein.

The terms “treat,” “treating” and “treatment,” as used herein, means reducing the frequency or severity with which symptoms of a disease or condition are experienced by a subject by virtue of administering an agent or compound to the subject.

### Lipid Compounds and Particles

In one aspect, the present disclosure provides a compound selected from the group consisting of:

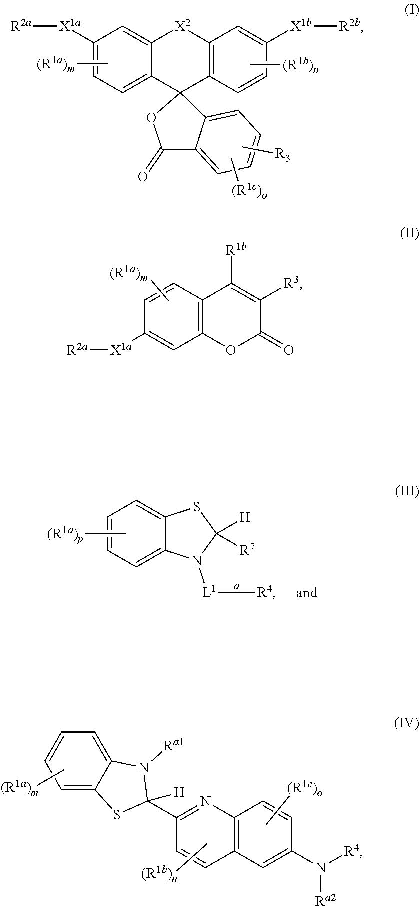

wherein:
R^{1a}, R^{1b}, R^{1c}, R^{1d}, R^{1e}, R^{1f}, and R^{1g}, if present, are each independently selected from the group consisting of optionally substituted C₁-C₆ alkyl, optionally substituted C₃-C₈ cycloalkyl, optionally substituted C₂-C₈ heterocycloalkyl, optionally substituted C₆-C₁₀ aryl, optionally substituted C₂-C₁₀ heteroaryl, halogen, CN, NO₂, OR^{A}, N(R^{A})(R^{B}), C(═O)OR^{A}, C(═O)N(R^{A})(R^{B}), S(═O)₂N(R^{A})(R^{B}), S(═O)N(R^{A})(R^{B}), OC(═O)R^{A}, and N(R^{A})C(═O)R^{B};
R^{2a} and R^{2b}, if present, are each independently selected from the group consisting of

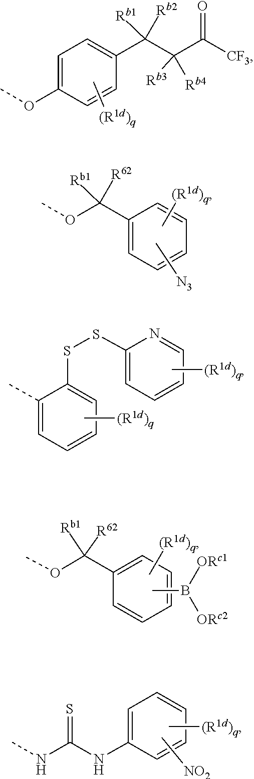

B(OR^{c1})(OR^{c2}), N(R^{A})(R^{B}), and OR^{A},
   wherein no more than one of R^{2a} and R^{2b} is N(R^{A})(R^{B}) or OR^{A}; R³, if present, is

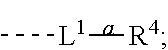

R⁴ is selected from the group consisting of optionally substituted C₁-C₃₀ alkyl, optionally substituted C₂-C₃₀ heteroalkyl, optionally substituted C₂-C₃₀ alkenyl, optionally substituted C₂-C₃₀ alkynyl,

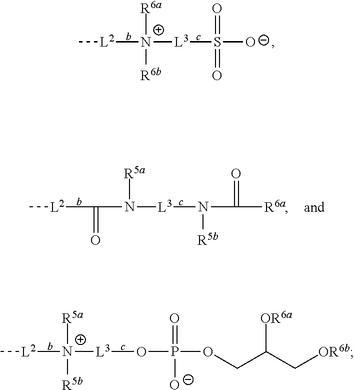

R^{5a} and R^{5b}, if present, are each independently selected from the group consisting of H, optionally substituted C₁-C₆ alkyl, optionally substituted C₃-C₈ cycloalkyl, optionally substituted C₆-C₁₀ aryl, and optionally substituted C₂-C₁₀ heteroaryl;
R^{6a} and R^{6b}, if present, are each independently selected from the group consisting of optionally substituted C₁-C₃₀ alkyl, optionally substituted C₂-C₃₀ heteroalkyl, optionally substituted C₂-C₃₀ alkenyl, optionally substituted C₂-C₃₀ alkynyl, C(═O)(optionally substituted C₁-C₃₀ alkyl), C(═O)(optionally substituted C₂-C₃₀ heteroalkyl), C(═O)(optionally substituted C₂-C₃₀ alkenyl), and C(═O)(optionally substituted C₂-C₃₀ alkynyl);
R⁷, if present, is

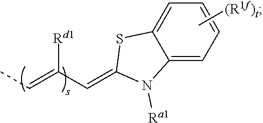

L¹, if present, is selected from the group consisting of

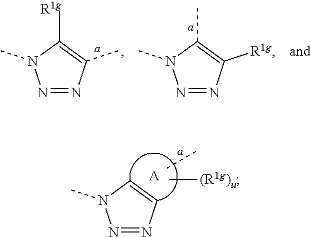

ring A, if present, is selected from the group consisting of C₅-C₁₂ cycloalkyl and C₂-C₁₀ heterocycloalkyl;
L² and L³ are each independently selected from the group consisting of optionally substituted C₁-C₁₂ alkylenyl, optionally substituted C₂-C₁₂ heteroalkylenyl, optionally substituted C₃-C₁₂ cycloalkylenyl, optionally substituted C₂-C₁₀ heterocycloalkylenyl, optionally substituted C₆-C₁₀ arylenyl, and optionally substituted C₂-C₁₂ heteroarylenyl;
X^{1a} and X^{1b}, if present, are each independently selected from the group consisting of a bond, —C(═O), —OC(═O)—, and —N(R^{A})C(═O)—;
X², if present, is selected from the group consisting of O and Si(R^{A})(R^{B});
R^{a1} and R^{a2} if present, are each independently selected from the group consisting of H, optionally substituted C₁-C₆ alkyl, C(═O)(optionally substituted C₁-C₆ alkyl), and hydrocarbyl;
R^{b1}, R^{b2}, R^{b3}, and R^{b4}, if present, are each independently selected from the group consisting of H and optionally substituted C₁-C₆ alkyl;
R^{c1} and R^{c2}, if present, are each independently selected from the group consisting of H and optionally substituted C₁-C₆ alkyl, or
   R^{c1} and R^{c2} combine with the atoms to which they are bound to form an optionally substituted C₂-C₈ heterocycloalkyl;
each occurrence of R^{d1}, if present, is independently selected from the group consisting of H and optionally substituted C₁-C₆ alkyl,
   wherein two R^{d1} groups having a 1,3-relationship can combine with the atoms to which they are bound to form an optionally substituted C₅-C₁₀ cycloalkenyl;
R^{A} and R^{B} are each independently selected from the group consisting of H, optionally substituted C₁-C₆ alkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₆-C₁₀ aryl, optionally substituted C₂-C₁₀ heteroaryl;
m, n, and o are each independently selected from the group consisting of 0, 1, 2, and 3;
p, q, r, and t are each independently selected from the group consisting of 0, 1, 2, 3, and 4;
s is selected from the group consisting of 1, 2, 3, and 4; and
u is selected from the group consisting of 0, 1, 2, 3, 4, 5, 6, 7, 8, 9, and 10;
or a salt, solvate, stereoisomer, or isotopologue thereof.

In certain embodiments, certain bonds are identified using a lower case letter (e.g., bond a, bond b, and bond c). In certain embodiments, bond

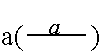

indicates a bond between L¹ and R⁴ (i.e.,

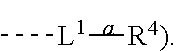

In certain embodiments, wherein R⁴ comprises

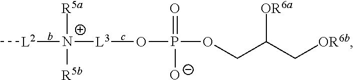

bond a indicates a bond between L¹ and L²

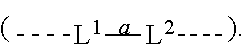

In certain embodiments, bond

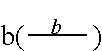

indicates a bond between L² and N in R⁴ (i.e.,

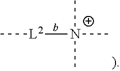

In certain embodiments, bond

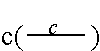

indicates a bond between L³ and O in R⁴ (i.e.,

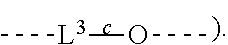

In certain embodiments, R⁴ is

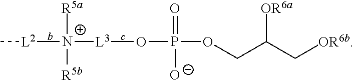

In certain embodiments, R⁴ is

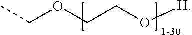

In certain embodiments, R⁴ is

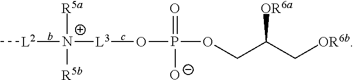

In certain embodiments, R⁴ is

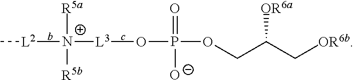

In certain embodiments, R⁴ is —(CH₂)₁₋₁₀C(═O)NH(CH₂)₂₋₁₀NHC(═O)(CH₂)₀₋₉CH₃. In certain embodiments, R⁴ is —(CH₂)₁₋₁₀N⁺[(CH₂)₀₋₉CH₃]₂(CH₂)₁₋₁₀S(═O)₂O⁻.

In certain embodiments, L² is —CH₂—. In certain embodiments, L² is —(CH₂)₂—. In certain embodiments, L² is

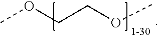

In certain embodiments, L² is

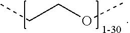

In certain embodiments, L³ is —CH₂—. In certain embodiments, L³ is —(CH₂)₂—. In certain embodiments, L³ is

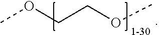

In certain embodiments, L³ is

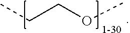

In certain embodiments, R^{5a} is CH₃. In certain embodiments, R^{5b} is CH₃.

In certain embodiments, R^{6a} is C(═O)(C₁₀-C₂₀ alkyl). In certain embodiments, R^{6a} is C(═O)(C₁₀-C₂₀ alkyl). In certain embodiments, R^{6a} is C(═O)(pentadecanyl). In certain embodiments, R^{6a} is C(═O)(pentadecanyl).

In certain embodiments, R⁴ is:

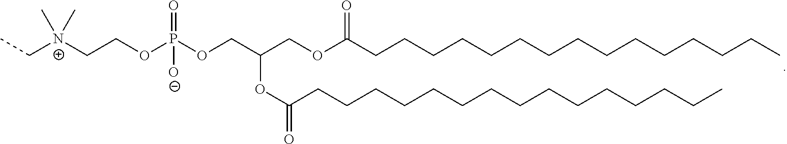

In certain embodiments, L¹ is

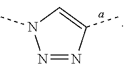

In certain embodiments, L¹ is

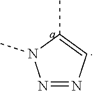

In certain embodiments, L¹ is

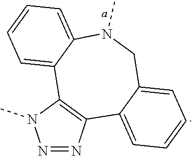

In certain embodiments, R^{2a} is B(OH)₂. In certain embodiments, R^{2a} is

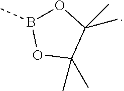

In certain embodiments, R^{2a} is

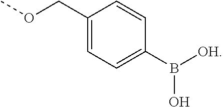

In certain embodiments, R^{2a} is

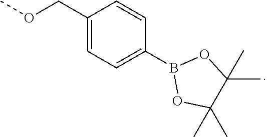

In certain embodiments, R^{2a} is

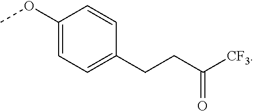

In certain embodiments, R^{2a} is

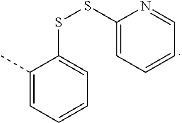

In certain embodiments, R^{2a} is

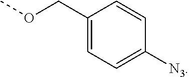

In certain embodiments, R^{2a} is

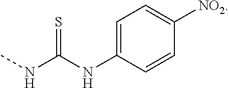

In certain embodiments, R^{2b} is B(OH)₂. In certain embodiments, R^{2b} is

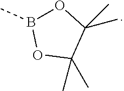

In certain embodiments, R^{2b} is

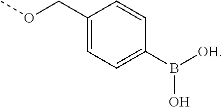

In certain embodiments, R^{2b} is

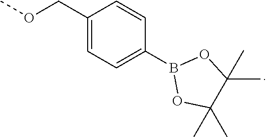

In certain embodiments, R^{2b} is

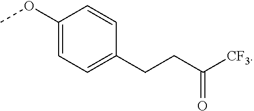

In certain embodiments, R^{2b} is

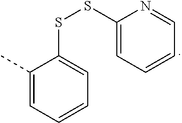

In certain embodiments, R^{2b} is

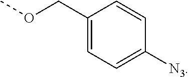

In certain embodiments, R^{2b} is

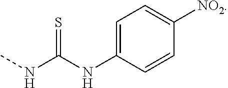

In certain embodiments, the compound is a compound of Formula (Ia):

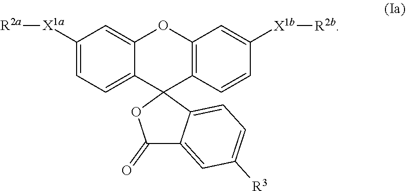

In certain embodiments, the compound is a compound of Formula (Ib):

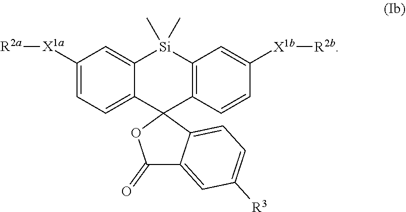

In certain embodiments, X^{1a} and X^{2a} combine to form

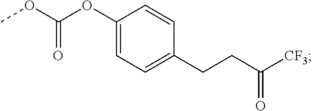

and X^{1b} and X^{2b} combine to form

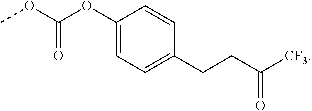

In certain embodiments, X^{1a} and X^{2a} combine to form

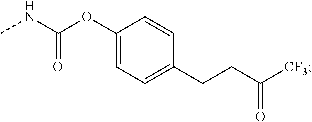

and X^{1b} and X^{2b} combine to form

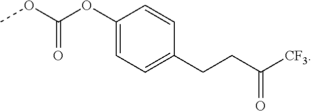

In certain embodiments, X^{1a} and X^{2a} combine to form

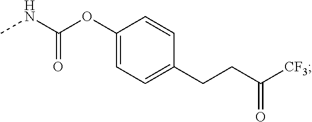

and X^{1b} and X^{2b} combine to form

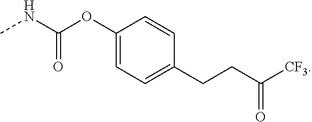

In certain embodiments, X^{1a} and X^{2a} combine to form

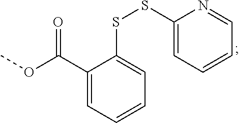

and X^{1b} and X^{2b} combine to form

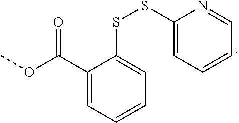

In certain embodiments, X^{1a} and X^{2a} combine to form

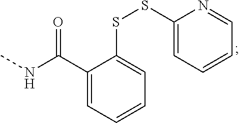

and X^{1b} and X^{2b} combine to form

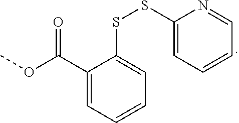

In certain embodiments, X^{1a} and X^{2a} combine to form

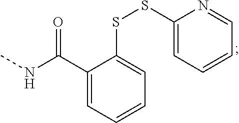

and X^{1b} and X^{2b} combine to form

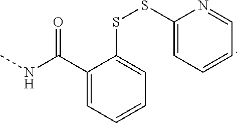

In certain embodiments, X^{1a} and X^{2a} combine to form

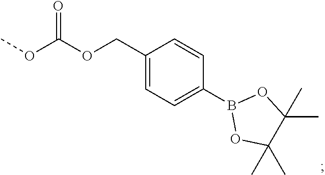

and X^{1b} and X^{2b} combine to form

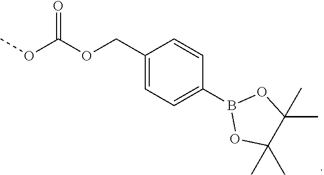

In certain embodiments, X^{1a} and X^{2a} combine to form

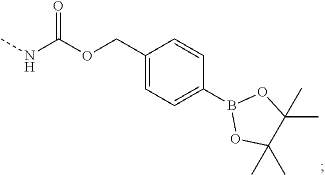

and X^{1b} and X^{2b} combine to form

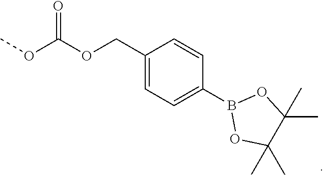

In certain embodiments, X^{1a} and X^{2a} combine to form

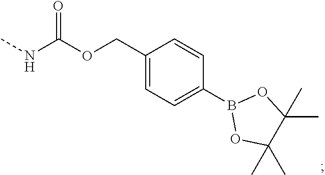

and X^{1b} and X^{2b} combine to form

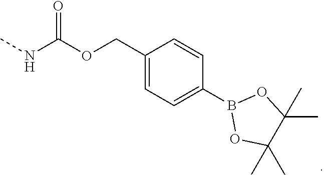

In certain embodiments, the compound is a compound of Formula (IIa):

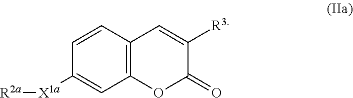

In certain embodiments, X^{1a} and X^{2a} combine to form

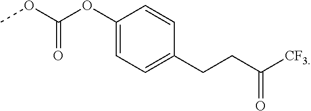

In certain embodiments, X^{1a} and X^{2a} combine to form

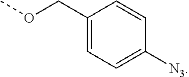

In certain embodiments, X^{1a} and X^{2a} combine to form

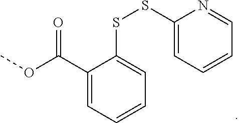

In certain embodiments, X^{1a} and X^{2a} combine to form

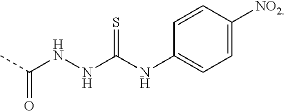

In certain embodiments, the compound is a compound of Formula (IIIa):

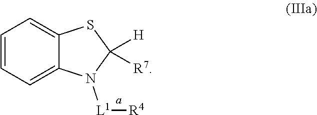

In certain embodiments, L¹ is —(CH₂)₂—.

In certain embodiments, R⁷ is

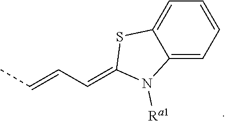

In certain embodiments, R⁷ is

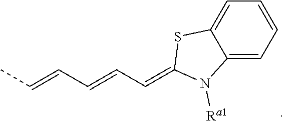

In certain embodiments, R⁷ is

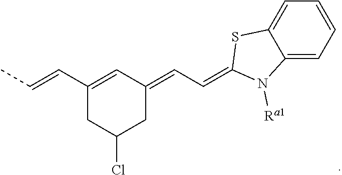

In certain embodiments, R^{a1} is H. In certain embodiments, R^{a1} is CH₃.

In certain embodiments, the compound is a compound of Formula (IVa):

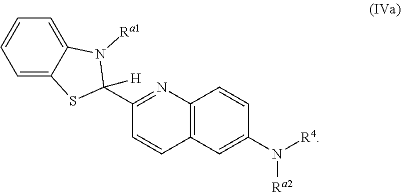

In certain embodiments, R^{a1} is H. In certain embodiments, R^{a1} is CH₃. In certain embodiments, R^{a2} is H. In certain embodiments, R^{a2} is CH₃.

In certain embodiments, the compound is at least one compound selected from Table 1.

TABLE 1
Exemplary masked fluorogenic lipid compounds
Compound

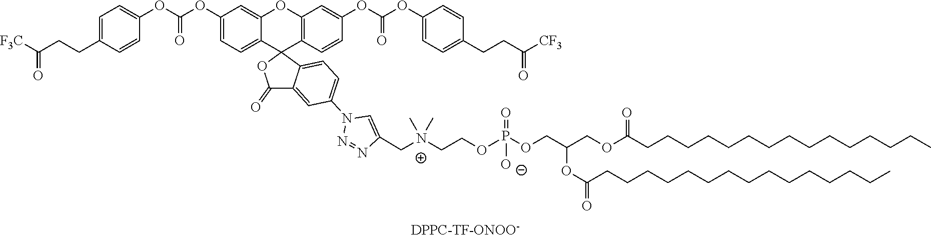 [SMILES: CCCCCCCCCCCCCCCC(=O)OCC(COP(=O)([O-])OCC[N+](C)(C)Cc1cn(-c2ccc3c(c2)C(=O)OC32c3ccc(OC(=O)Oc4ccc(CCC(=O)C(F)(F)F)cc4)cc3Oc3cc(OC(=O)Oc4ccc(CCC(=O)C(F)(F)F)cc4)ccc32)nn1)OC(=O)CCCCCCCCCCCCCCC ; "DPPC-TF-ONOO-"]

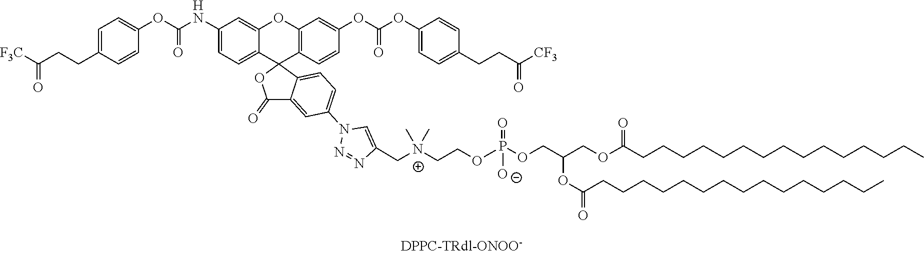 [SMILES: CCCCCCCCCCCCCCCC(=O)OCC(COP(=O)([O-])OCC[N+](C)(C)Cc1cn(-c2ccc3c(c2)C(=O)OC32c3ccc(NC(=O)Oc4ccc(CCC(=O)C(F)(F)F)cc4)cc3Oc3cc(OC(=O)Oc4ccc(CCC(=O)C(F)(F)F)cc4)ccc32)nn1)OC(=O)CCCCCCCCCCCCCCC]

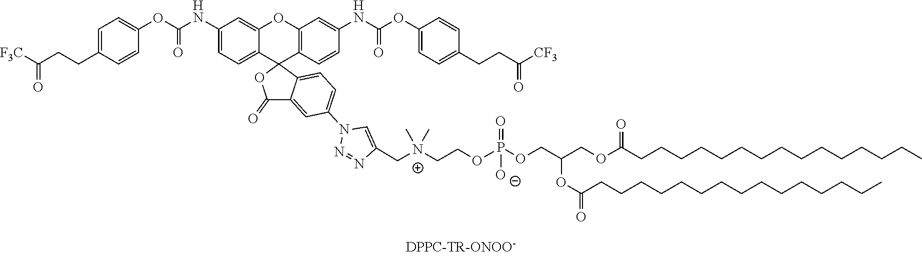 [SMILES: CCCCCCCCCCCCCCCC(=O)OCC(COP(=O)([O-])OCC[N+](C)(C)Cc1cn(-c2ccc3c(c2)C(=O)OC32c3ccc(NC(=O)Oc4ccc(CCC(=O)C(F)(F)F)cc4)cc3Oc3cc(NC(=O)Oc4ccc(CCC(=O)C(F)(F)F)cc4)ccc32)nn1)OC(=O)CCCCCCCCCCCCCCC]

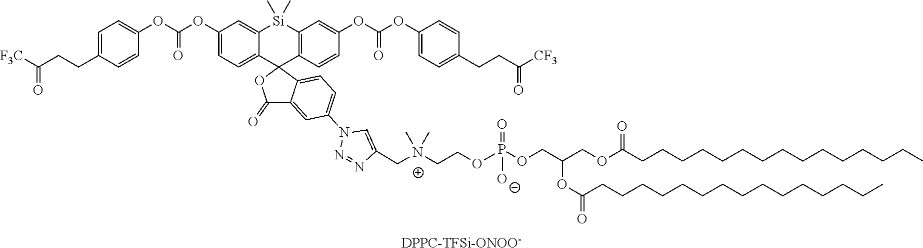 [SMILES: CCCCCCCCCCCCCCCC(=O)OCC(COP(=O)([O-])OCC[N+](C)(C)Cc1cn(-c2ccc3c(c2)C(=O)OC32c3ccc(OC(=O)Oc4ccc(CCC(=O)C(F)(F)F)cc4)cc3[Si](C)(C)c3cc(OC(=O)Oc4ccc(CCC(=O)C(F)(F)F)cc4)ccc32)nn1)OC(=O)CCCCCCCCCCCCCCC ; "DPPC-TFSi-ONOO-"]

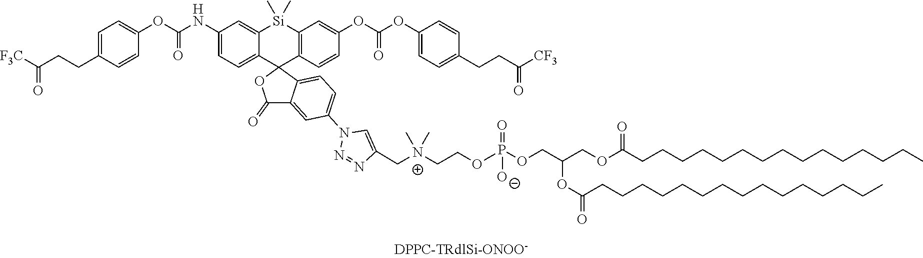 [SMILES: CCCCCCCCCCCCCCCC(=O)OCC(COP(=O)([O-])OCC[N+](C)(C)Cc1cn(-c2ccc3c(c2)C(=O)OC32c3ccc(NC(=O)Oc4ccc(CCC(=O)C(F)(F)F)cc4)cc3[Si](C)(C)c3cc(OC(=O)Oc4ccc(CCC(=O)C(F)(F)F)cc4)ccc32)nn1)OC(=O)CCCCCCCCCCCCCCC]

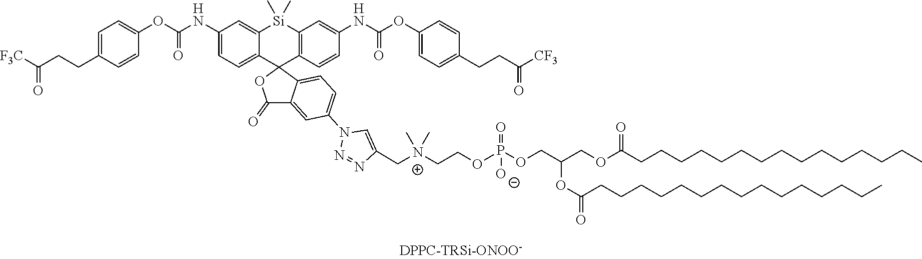 [SMILES: CCCCCCCCCCCCCCCC(=O)OCC(COP(=O)([O-])OCC[N+](C)(C)Cc1cn(-c2ccc3c(c2)C(=O)OC32c3ccc(NC(=O)Oc4ccc(CCC(=O)C(F)(F)F)cc4)cc3[Si](C)(C)c3cc(NC(=O)Oc4ccc(CCC(=O)C(F)(F)F)cc4)ccc32)nn1)OC(=O)CCCCCCCCCCCCCCC]

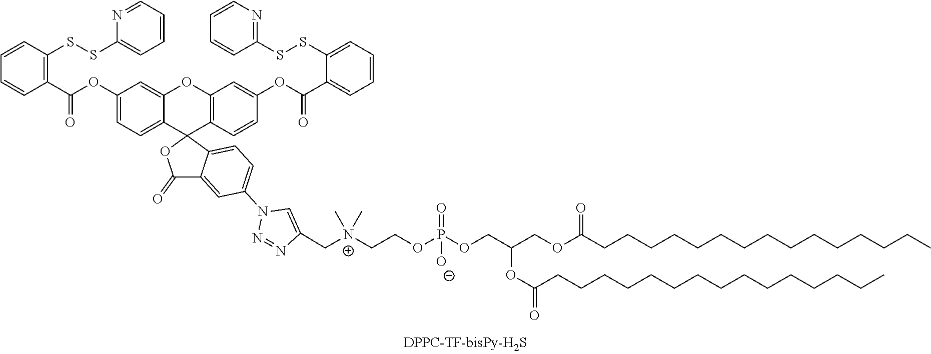 [SMILES: CCCCCCCCCCCCCCCC(=O)OCC(COP(=O)([O-])OCC[N+](C)(C)Cc1cn(-c2ccc3c(c2)C(=O)OC32c3ccc(OC(=O)c4ccccc4SSc4ccccn4)cc3Oc3cc(OC(=O)c4ccccc4SSc4ccccn4)ccc32)nn1)OC(=O)CCCCCCCCCCCCCCC]

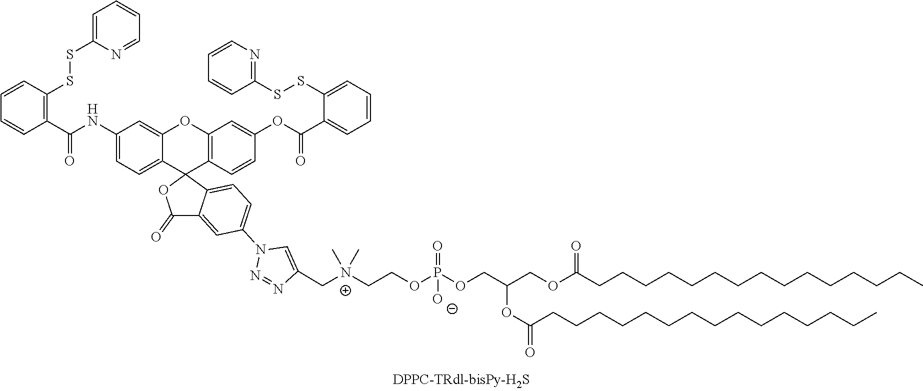 [SMILES: CCCCCCCCCCCCCCCC(=O)OCC(COP(=O)([O-])OCC[N+](C)(C)Cc1cn(-c2ccc3c(c2)C(=O)OC32c3ccc(NC(=O)c4ccccc4SSc4ccccn4)cc3Oc3cc(OC(=O)c4ccccc4SSc4ccccn4)ccc32)nn1)OC(=O)CCCCCCCCCCCCCCC]

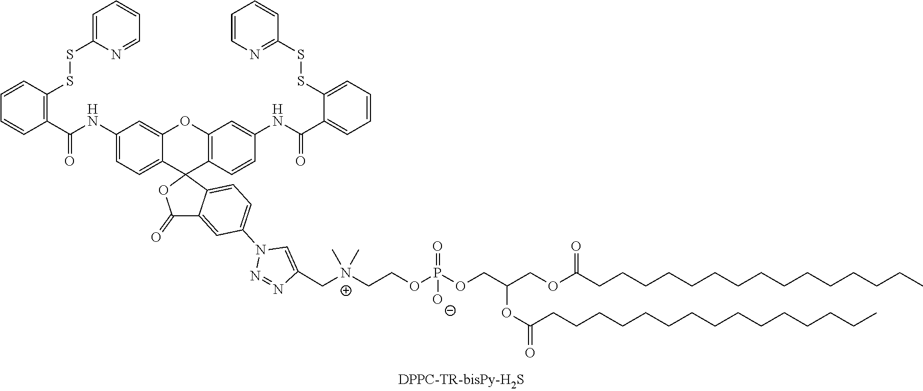 [SMILES: CCCCCCCCCCCCCCCC(=O)OCC(COP(=O)([O-])OCC[N+](C)(C)Cc1cn(-c2ccc3c(c2)C(=O)OC32c3ccc(NC(=O)c4ccccc4SSc4ccccn4)cc3Oc3cc(NC(=O)c4ccccc4SSc4ccccn4)ccc32)nn1)OC(=O)CCCCCCCCCCCCCCC]

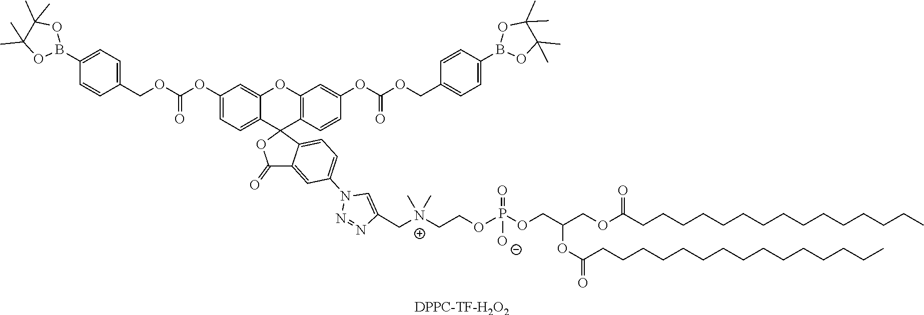 [SMILES: CCCCCCCCCCCCCCCC(=O)OCC(COP(=O)([O-])OCC[N+](C)(C)Cc1cn(-c2ccc3c(c2)C(=O)OC32c3ccc(OC(=O)OCc4ccc(B5OC(C)(C)C(C)(C)O5)cc4)cc3Oc3cc(OC(=O)OCc4ccc(B5OC(C)(C)C(C)(C)O5)cc4)ccc32)nn1)OC(=O)CCCCCCCCCCCCCCC ; "DPPC-TF-H2O2"]

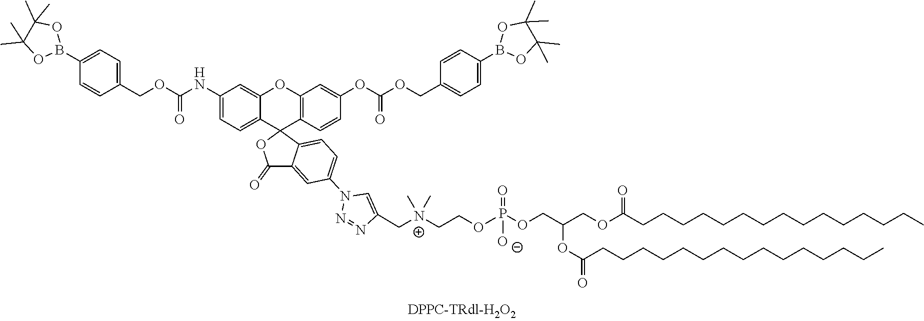 [SMILES: CCCCCCCCCCCCCCCC(=O)OCC(COP(=O)([O-])OCC[N+](C)(C)Cc1cn(-c2ccc3c(c2)C(=O)OC32c3ccc(NC(=O)OCc4ccc(B5OC(C)(C)C(C)(C)O5)cc4)cc3Oc3cc(OC(=O)OCc4ccc(B5OC(C)(C)C(C)(C)O5)cc4)ccc32)nn1)OC(=O)CCCCCCCCCCCCCCC]

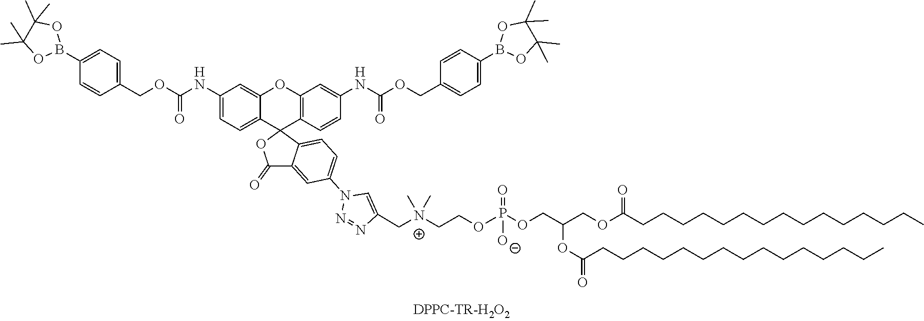 [SMILES: CCCCCCCCCCCCCCCC(=O)OCC(COP(=O)([O-])OCC[N+](C)(C)Cc1cn(-c2ccc3c(c2)C(=O)OC32c3ccc(NC(=O)OCc4ccc(B5OC(C)(C)C(C)(C)O5)cc4)cc3Oc3cc(NC(=O)OCc4ccc(B5OC(C)(C)C(C)(C)O5)cc4)ccc32)nn1)OC(=O)CCCCCCCCCCCCCCC]

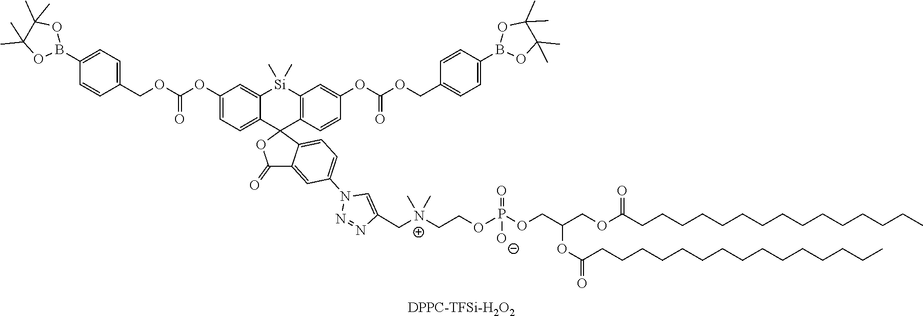 [SMILES: CCCCCCCCCCCCCCCC(=O)OCC(COP(=O)([O-])OCC[N+](C)(C)Cc1cn(-c2ccc3c(c2)C(=O)OC32c3ccc(OC(=O)OCc4ccc(B5OC(C)(C)C(C)(C)O5)cc4)cc3[Si](C)(C)c3cc(OC(=O)OCc4ccc(B5OC(C)(C)C(C)(C)O5)cc4)ccc32)nn1)OC(=O)CCCCCCCCCCCCCCC ; "DPPC-TFSi-H2O2"]

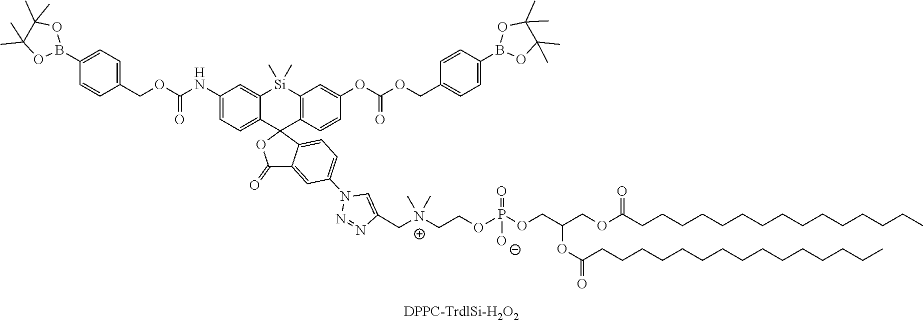 [SMILES: CCCCCCCCCCCCCCCC(=O)OCC(COP(=O)([O-])OCC[N+](C)(C)Cc1cn(-c2ccc3c(c2)C(=O)OC32c3ccc(NC(=O)OCc4ccc(B5OC(C)(C)C(C)(C)O5)cc4)cc3[Si](C)(C)c3cc(OC(=O)OCc4ccc(B5OC(C)(C)C(C)(C)O5)cc4)ccc32)nn1)OC(=O)CCCCCCCCCCCCCCC]

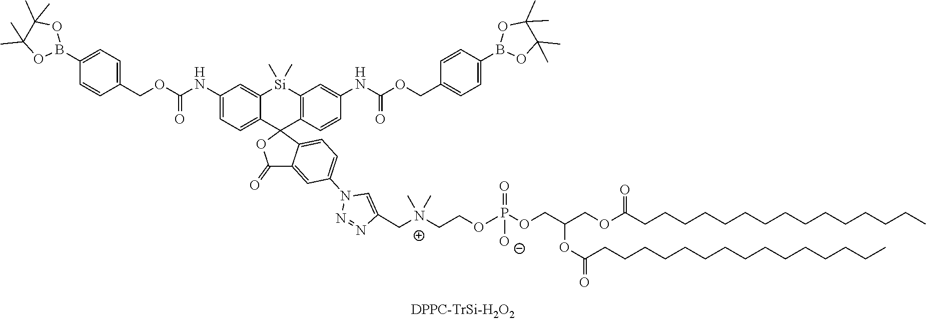 [SMILES: CCCCCCCCCCCCCCCC(=O)OCC(COP(=O)([O-])OCC[N+](C)(C)Cc1cn(-c2ccc3c(c2)C(=O)OC32c3ccc(NC(=O)OCc4ccc(B5OC(C)(C)C(C)(C)O5)cc4)cc3[Si](C)(C)c3cc(NC(=O)OCc4ccc(B5OC(C)(C)C(C)(C)O5)cc4)ccc32)nn1)OC(=O)CCCCCCCCCCCCCCC]

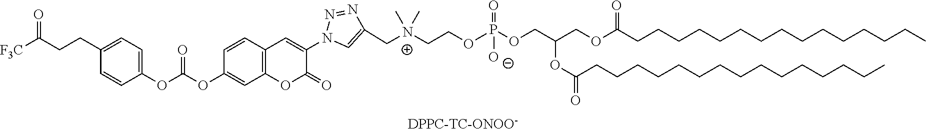 [SMILES: CCCCCCCCCCCCCCCC(=O)OCC(COP(=O)([O-])OCC[N+](C)(C)Cc1cn(-c2cc3ccc(OC(=O)Oc4ccc(CCC(=O)C(F)(F)F)cc4)cc3oc2=O)nn1)OC(=O)CCCCCCCCCCCCCCC ; "DPPC-TC-ONOO-"]

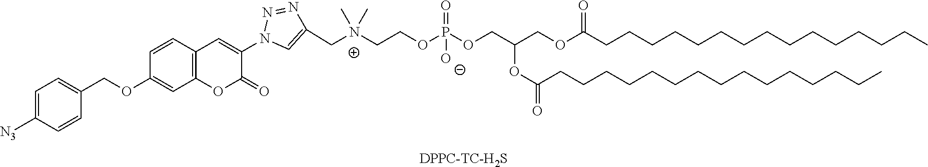 [SMILES: CCCCCCCCCCCCCCCC(=O)OCC(COP(=O)([O-])OCC[N+](C)(C)Cc1cn(-c2cc3ccc(OCc4ccc(N=[N+]=[N-])cc4)cc3oc2=O)nn1)OC(=O)CCCCCCCCCCCCCCC ; "DPPC-TC-H2S"]

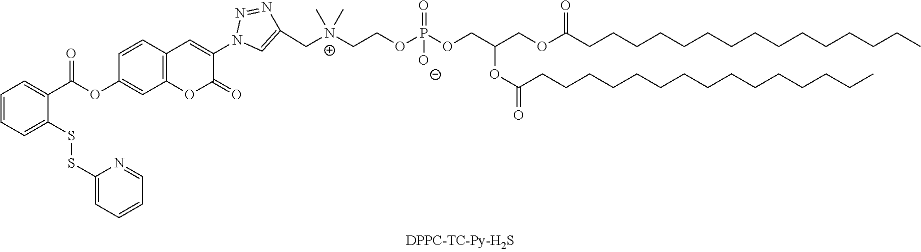 [SMILES: CCCCCCCCCCCCCCCC(=O)OCC(COP(=O)([O-])OCC[N+](C)(C)Cc1cn(-c2cc3ccc(OC(=O)c4ccccc4SSc4ccccn4)cc3oc2=O)nn1)OC(=O)CCCCCCCCCCCCCCC]

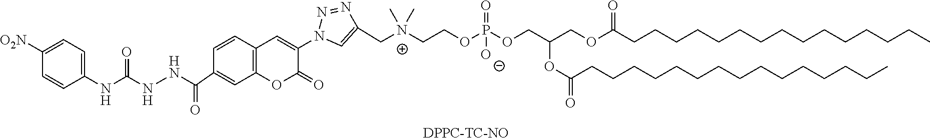 [SMILES: CCCCCCCCCCCCCCCC(=O)OCC(COP(=O)([O-])OCC[N+](C)(C)Cc1cn(-c2cc3ccc(C(=O)NNC(=O)Nc4ccc([N+](=O)[O-])cc4)cc3oc2=O)nn1)OC(=O)CCCCCCCCCCCCCCC ; "DPPC-TC-NO"]

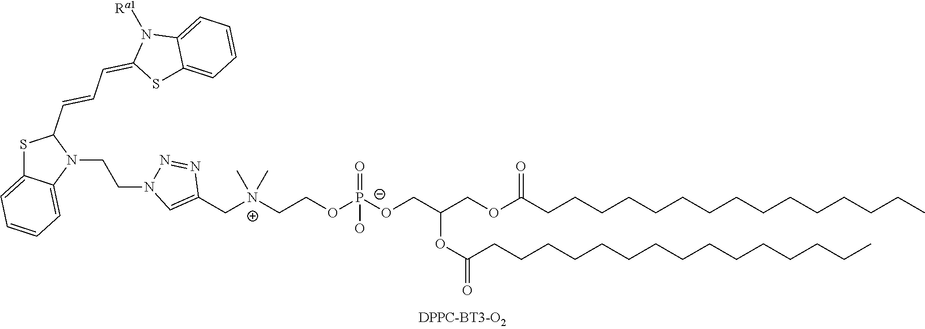

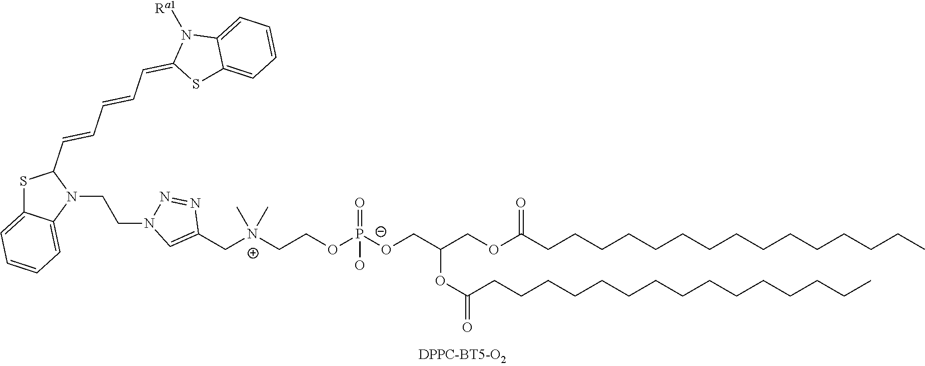

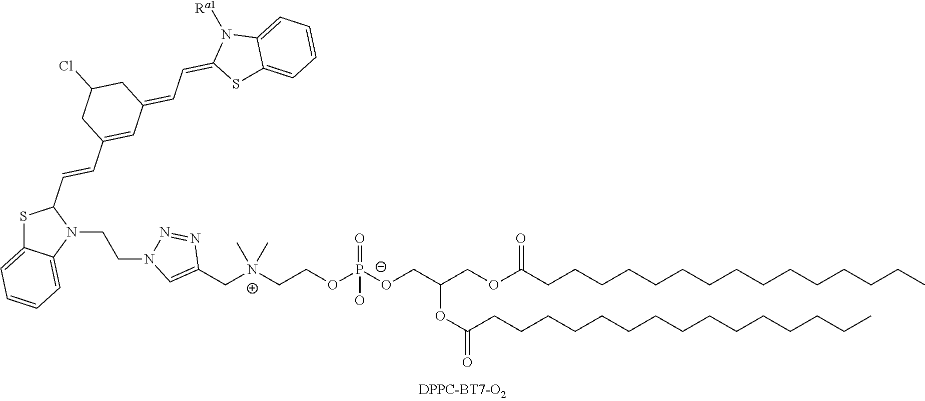

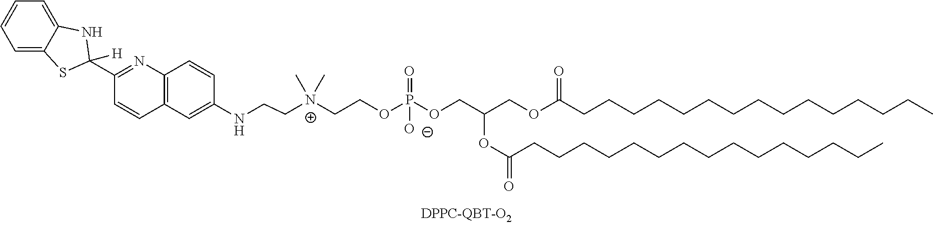 [SMILES: CCCCCCCCCCCCCCCC(=O)OCC(COP(=O)([O-])OCC[N+](C)(C)CCNc1ccc2nc(C3Nc4ccccc4S3)ccc2c1)OC(=O)CCCCCCCCCCCCCCC]

In one aspect, the present disclosure provides a lipid particle comprising:
(a) at least one compound of the present disclosure; and
(b) at least one additional lipid.

In certain embodiments, the lipid particle is a giant vesicle. In certain embodiments, the lipid particle is a nanoparticle.

In certain embodiments, (a) and (b) have a molar ratio ranging from about of about 1:99, 2:98, 3:97, 4:96, 5:95, 6:94, 7:93, 8:92, 9:91, or about 10:90.

In certain embodiments, the at least one additional lipid comprises at least one selected from the group consisting of a phospholipid and cholesterol, or a modified derivative thereof.

In certain embodiments, the at least one additional lipid comprises at least one selected from the group consisting of 1-palmitoyl-2-oleoyl-glycero-3-phosphocholine (POPC), 1-hexadecanoyl-2-(9Z-octadecenoyl)-sn-glycero-3-phosphoglycerol (POPG), 1,2-di-O-octadecenyl-3-trimethylammonium propane (DOTMA), 1,2-dioleoyl-sn-glycero-3-phosphoethanolamine (DOPE), and cholesterol.

### Methods

In one aspect, the present disclosure provides a method of detecting a redox biomarker in a sample, the method comprising:
(a) contacting at least one compound of the present disclosure and/or at least one lipid particle of the present disclosure with the sample, so as to provide a mixture; and
(b) detecting a fluorescent signal in the mixture, wherein the fluorescent signal is indicative of a redox biomarker in the sample.

In certain embodiments, the sample is a biological sample obtained from a subject.

In certain embodiments, the redox biomarker is selected from the group consisting of peroxynitrite (ONOO⁻), hydrogen sulfide (H₂S), hydrogen peroxide (H₂O₂), superoxide (O₂·⁻), and nitric oxide (NO·).

In certain embodiments, the contacting of the compound any of Formulae (I)—(IV) and the redox biomarker in the sample results in chemical modification of the compound of any of Formulae (I)—(IV).

In another aspect, the present disclosure provides a method of diagnosing a disease or disorder associated with oxidative, nitrosative, or nitrative stress in a subject, the method comprising:
(a) contacting at least one compound of the present disclosure and/or at least one lipid particle of the present disclosure with a sample obtained from the subject to provide a mixture; and
(b) detecting a fluorescent signal in the mixture, wherein the fluorescent signal is indicative of a disease or disorder associated with oxidative, nitrosative, or nitrative stress.

In certain embodiments, the disease or disorder associated with oxidative, nitrosative, or nitrative stress is characterized by an elevated concentration of at least one redox biomarker in at least one bodily fluid.

In certain embodiments, the redox biomarker is at least one selected from the group consisting of peroxynitrite (ONOO⁻), hydrogen sulfide (H₂S), hydrogen peroxide (H₂O₂), superoxide (O₂·⁻), and nitric oxide (NO·).

In certain embodiments, the contacting of the compound of the present disclosure with the sample obtained from the subject results in chemical modification of the compound of the present disclosure.

In certain embodiments, the disease or disorder is at least one selected from the group consisting of cancer, diabetes, chronic inflammatory disease or disorder, pulmonary disease or disorder, cardiovascular disease or disorder, neurodegenerative disease or disorder, liver disease or disorder, kidney disease or disorder, and metabolic disease or disorder.

In certain embodiments, the method further comprises administering to the subject at least one compound suitable to treat the disease or disorder associated with oxidative, nitrosative, or nitrative stress.

### EXAMPLES

Various embodiments of the present application can be better understood by reference to the following Examples which are offered by way of illustration. The scope of the present application is not limited to the Examples given herein.

### Example 1: Redox Agent (e.g., Peroxynitrite, Hydrogen Sulfide, and Hydrogen Peroxide) Sensing Phospholipids and Amphiphilic Analogues Thereof

### Peroxynitrite (ONOO⁻)

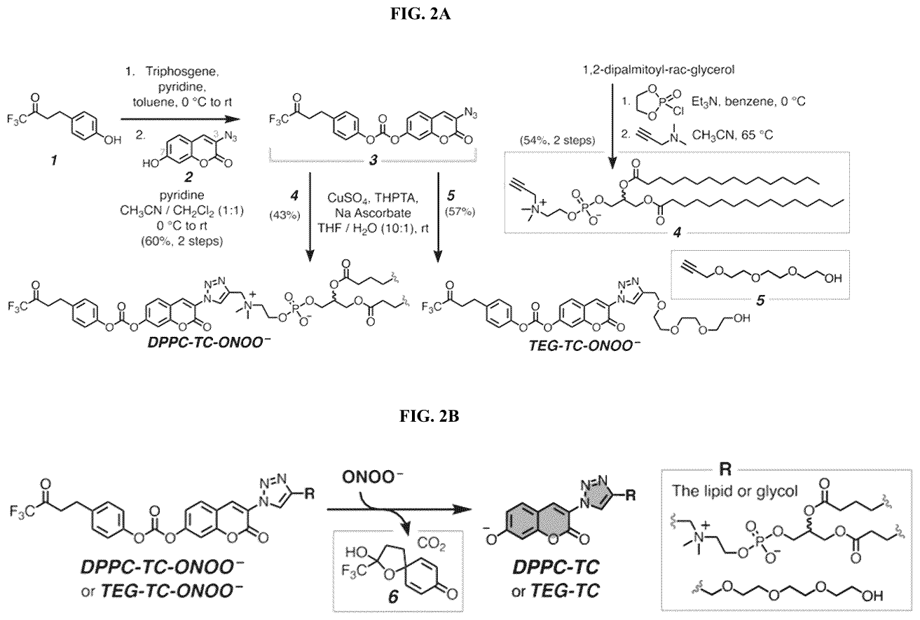

A concise route was developed for the synthesis of peroxynitrite-sensing phospholipid, DPPC-TC-ONOO⁻ (FIG. 2A), which includes 1,2-dipalmitoyl-rac-glycero-3-phosphocholine (rac-DPPC) as the phospholipid framework, 3-triazole-coumarin as the fluorophore, and p-(4,4,4-trifluoro-3-oxobutyl)phenyl as the peroxynitrite-reactive module. In addition, to facilitate investigations of the peroxynitrite-mediated florescence activation mechanism and fidelity, the non-amphiphilic analog TEG-TC-ONOO⁻ was synthesized, which contains a triethylene glycol (TEG) moiety in place of rac-DPPC (FIGS. 2A-2B). TEG-TC-ONOO⁻ exists in a non-aggregated state in aqueous media, making the spectroscopic and spectrophotometric analyses of its redox reactions straightforward. In addition, TEG-TC-ONOO⁻ allows a comparison between the amphiphilic and non-amphiphilic probes in terms of subcellular localization.

For both DPPC-TC-ONOO⁻ and TEG-TC-ONOO⁻, the p-(4,4,4-trifluoro-3-oxobutyl)phenyl and 3-triazole-7-hydroxycoumarin groups were connected through a carbonate linkage. It was reasoned that the carbonate linkage could mask 7-0 of the coumarin, diminishing its fluorescence intensity. In the presence of ONOO−, the p-(4,4,4-trifluoro-3-oxobutyl)phenyl moiety would undergo an oxidative spirocyclization. Without wishing to be bound by any theory, it was hypothesized that water could add to the resulting carbonate by nucleophilic addition, eliminating oxa-spiro[4,5] decenone 6a and subsequently uncaging the coumarin motif via decarboxylation to afford either DPPC-TC or TEG-TC (FIG. 2B).

Both DPPC-TC-ONOO⁻ and TEG-TC-ONOO⁻ were synthesized through convergent routes in which the longest linear sequence required three chemical steps. First, 1,1,1-trifluoro-4-(4-hydroxyphenyl)butan-2-one (1a) was converted to its chloroformate using triphosgene and successively connected with 3-azido-7-hydroxycoumarin (2a) to furnish the carbonate 3a in 60% isolated yield over two steps. This peroxynitrite-sensitive compound served as a common intermediate in the construction of DPPC-TC-ONOO⁻ and TEG-TC-ONOO⁻. Through the copper-catalyzed azide alkyne cycloaddition reaction, the carbonate 3a was conjugated with either the propargylated rac-DPPC 4a or propargyl-TEG-OH 5 to afford DPPC-TC-ONOO⁻ (43%) or TEG-TC-ONOO⁻ (57%), respectively. To obtain the alkynyl lipid 4a, 1,2-dipalmitoyl-rac-glycerol was converted to its cyclic phosphate triester using ethylene chlorophosphite and opened the subsequent ring by the nucleophilic addition of 3-dimethylamino-1-propyne.

Hydrogen Sulfide (H₂S) and Hydrogen Peroxide (H₂O₂)

Both DPPC-TC-H₂S and DPPC-TF-H₂O₂ possess a modular design in which the sensor module, caged coumarin or fluorescein, is connected to rac-DPPC with a triazole ring (FIG. 3). In addition, to facilitate quantitative investigations of the redox-mediated fluorescence activation mechanism, kinetics, and fidelity, the non-amphiphilic analogs TEG-TC-H₂S and TEG-TF-H₂O₂ were synthesized, which contain a triethylene glycol (TEG) in place of the 1,2-dipalmitoyl-rac-glycero-3-phosphocholine (rac-DPPC) module to enhance water solubility. The syntheses of all probes make use of one of two building blocks, the propargylated rac-DPPC 1b or the readily available propargyl-TEG-OH 2b. To obtain 1b, 1,2-dipalmitoyl-rac-glycerol was converted to its cyclic phosphate triester using ethylene chlorophosphite and opened the subsequent phosphate ring via nucleophilic addition of 3-dimethylamino-1-propyne.

Through convergent routes, the amphiphilic and non-amphiphilic redox probes were synthesized in three or four longest linear sequences. The H₂S probe precursors DPPC-TC and TEG-TC, which are also the H₂S-mediated redox products of DPPC-TC-H₂S and TEG-TC-H₂S, were obtained from 3-azido-7-hydroxycoumarin (4b) via copper-catalyzed azide-alkyne cycloaddition (CuAAC) reaction with the respective conjugation partner, 1b or 2b. These precursors were then alkylated at their 7-0 atom using 4-azidobenzyl bromide (3b) to afford DPPC-TC-H₂S and TEG-TC-H₂S. The H₂O₂ probes DPPC-TF-H₂O₂ and TEG-TF-H₂O₂ were synthesized in three steps from the azido dicarbonate 7b. First, 4-hydroxymethylphenyl-boronic acid pinacol ester was converted to its chloroformate 5b using triphosgene and successively connected with 5-azidofluorescein (6b), obtained from 5-aminofluorescein, to furnish the azido dicarbonate 7b in 46% isolated yield. This molecule served as a common intermediate for both DPPC-TF-H₂O₂ and TEG-TF-H₂O₂ via CuAAC reaction with the propargylated rac-DPPC 1b and propargyl-TEG-OH 2b, respectively.

### Example 2: Redox Sensing Occurs Via Oxidative Uncaging of the Coumarin or Fluorescein Moieties

### Peroxynitrite (ONOO⁻)

To gain a mechanistic insight into the reaction of the probes with ONOO⁻, liquid chromatography was conducted, followed by high-resolution mass spectroscopy (LC-HRMS). The amphiphilic probe, DPPC-TC-ONOO⁻, displayed poor electrospray ionization profile, while TEG-TC-ONOO⁻ provided evidence for the formation of its oxidative decarboxylation product, TEG-TC upon mixing with ONOO⁻. Further, the progressive formation of the oxa-spiro[4,5] decenone 6a was observed, supporting the proposed oxidative elimination process (FIG. 2B).

The pKₐ of TEG-TC was determined to be 7.1±0.1 using UV-Vis spectrophotometric measurements. Under the confocal imaging conditions (pH 7.5-8.5 with cell-free assays, pH 7.4 with cells), a substantial population of the 7-hydroxycoumarin should exist in its aryloxide form. The change in coumarin fluorogenicity was assessed upon uncaging the probes from the oxobutylphenyl carbonate by measuring the relative fluorescence quantum yield (Φ_{Frel}) values (Table 2). Coumarin 343 was used as a standard, which has an absolute fluorescence quantum yield (Φ_{Frel}) of 0.63 in ethanol. The Φ_{Frel} values of TEG-TC-ONOO⁻ (entry 1) and DPPC-TC-ONOO⁻ (entry 3) were measured as 0.08 and 0.09, respectively. Each with a relatively low Φ_{Frel} and molar extinction coefficient, the probes were validated to exhibit weak brightness as a consequence of their coumarin 7-O-atom being derivatized as the carbonate. The Φ_{Frel} values for TEG-TC (entry 2) and DPPC-TC (entry 4), which were synthesized independently, were measured as 0.66 and 0.64, respectively. Taken together, these results suggested that conversion of the caged coumarins to their uncaging products enhances the fluorescence quantum yield by ≥7-fold and brightness by ˜30-fold.

TABLE 2
Characterization of exemplary peroxynitrite probes and uncaged forms thereof

| | Exₘₐₓ | Emₘₐₓ | Ext. Coeff. | Quantum | | Relative |
|---|---|---|---|---|---|---|
| Compound | (nm) | (nm) | (M⁻¹ · cm⁻¹) | Yield | Brightness | Brightness |
| TEG-TC-ONOO⁻ | 400.0 | 472.0 | 4475 | 0.08 | 358 | 1.4 |
| TEG-TC | 397.0 | 471.4 | 15613 | 0.66 | 10305 | 41.1 |
| DPPC-TC-ONOO⁻ | 395.0 | 471.2 | 2784 | 0.09 | 251 | 1.0 |
| DPPC-TC | 398.0 | 472.0 | 13749 | 0.64 | 8799 | 35.1 |

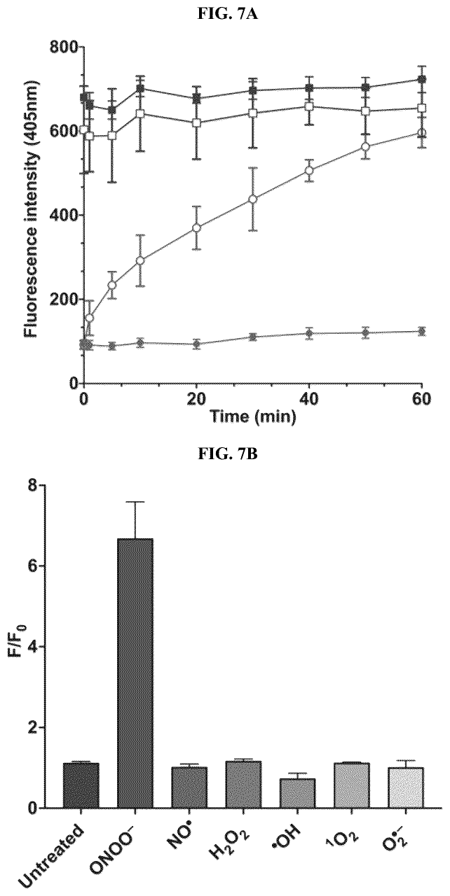

The response rate and magnitude of TEG-TC-ONOO⁻ against ONOO⁻ was evaluated by measuring the relative fluorescence intensities of samples containing TEG-TC-ONOO⁻ and TEG-TC (positive control) at 405 nm excitation (FIG. 7A). These samples, prepared in Tris buffer (50 mM, pH 7.5), were measured over 60 minutes of incubation with and without ONOO⁻. Physiologically normal ONOO⁻ production rate in cells has been estimated to be in the single-digit μM range, while reaching up to 100 μM under dysregulated conditions. Guided by this information, and in agreement with the fluorescence titration conditions employed for cytosolic resorufin-based peroxynitrite sensors, it was reasoned that administering 600 μM of ONOO⁻ into a pH 7.5 medium would provide a steady level of effective ONOO⁻ within a biologically relevant concentration range of −10-20 μM/min. Upon addition of ONOO⁻ (600 PM), the sample containing TEG-TC-ONOO⁻ exhibited a rapid increase in fluorescence, reaching ˜3-fold higher fluorescence within the first 10 minutes compared to the sample untreated. The difference in fluorescence between the peroxynitrite-treated and untreated samples reached up to 5-fold at the end of 60 minutes. During this time period, the relative fluorescence intensity of the sample containing TEG-TC-ONOO⁻ became ˜85% of that of the sample containing TEG-TC (positive control). This trend of increasing fluorescence output was mirrored in the level of conversion of TEG-TC-ONOO⁻ into TEG-TC based on UV-Vis spectrophotometric measurements.

The peroxynitrite-specificity of the probe design was assessed by subjecting TEG-TC-ONOO⁻ to other redox species, including NO·, hydrogen peroxide (H₂O₂), singlet oxygen (¹O₂), hydroxyl radicals (HO·), and O₂·⁻ (FIG. 7B). Change in fluorescence intensity following excitation at 405 nm was measured over 60 minutes of incubation of TEG-TC-ONOO⁻ with each species and presented based on the signal-to-background ratio F/F₀, where F and F₀ are defined as the fluorescence with and without the analyte. In the absence of a redox species, F/F₀ for TEG-TC-ONOO⁻ increased only 13% (F/F₀=1.13) over 60 minutes. Treatment of TEG-TC-ONOO⁻ with ONOO⁻ gave rise to ˜570% increase in fluorescence (F/F₀=6.69).

Notably, incubating TEG-TC-ONOO⁻ with species other than ONOO⁻ resulted in only negligible levels of change in fluorescence. NO·, a free radical RNS that serves as a signaling molecule in cells, had essentially no effect on fluorescence intensity (F/F₀=1.03). The most significant fluorescence enhancement was observed for H₂O₂, with a fluorescence intensity 16% stronger than that of the background (F/F₀=1.16). These results indicate that the relative fluorescence enhancement of TEG-TC-ONOO⁻ is nearly 36-fold for ONOO⁻ compared to the other RNS and ROS tested in this study.

Hydrogen Sulfide (H₂S) and Hydrogen Peroxide (H₂O₂)

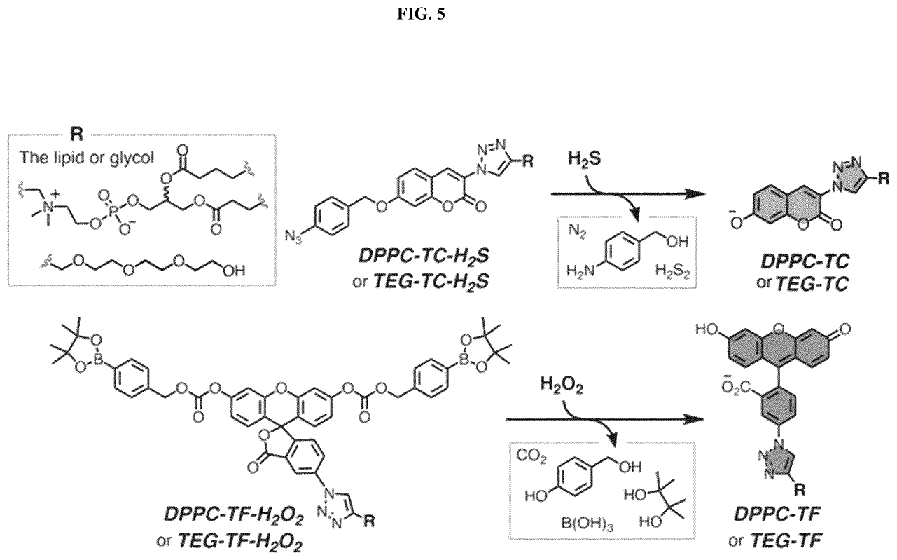

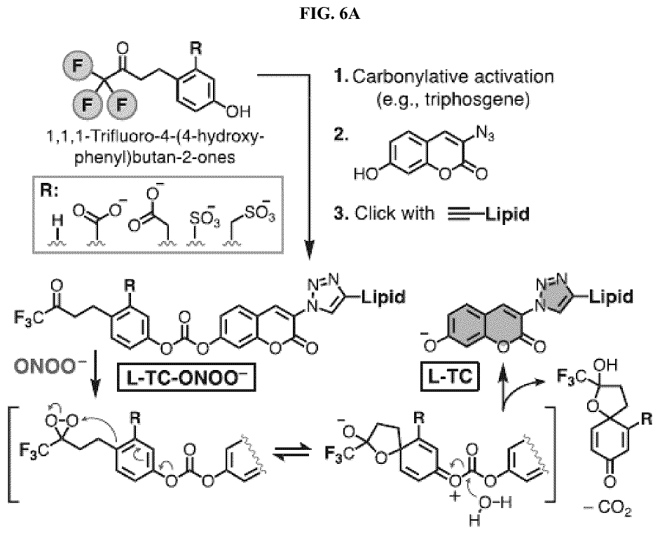

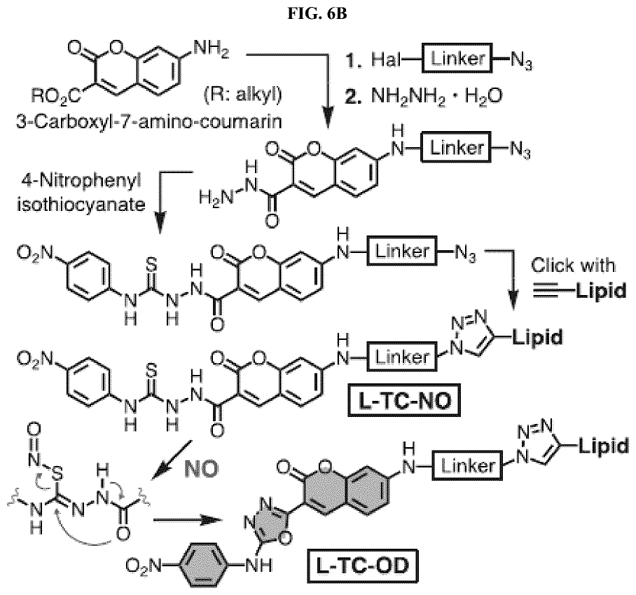

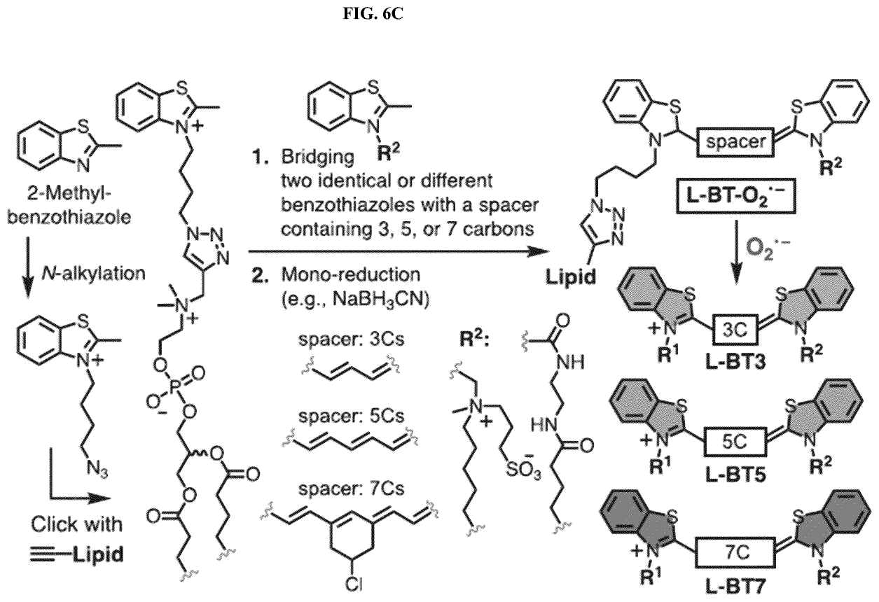

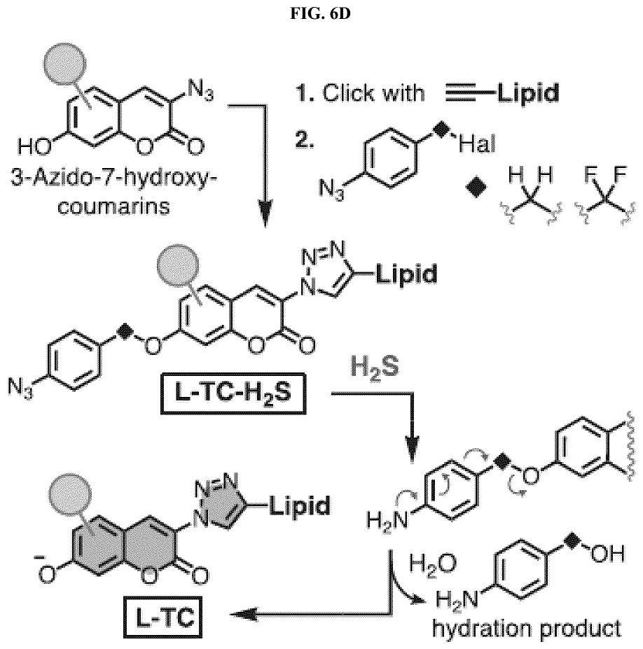

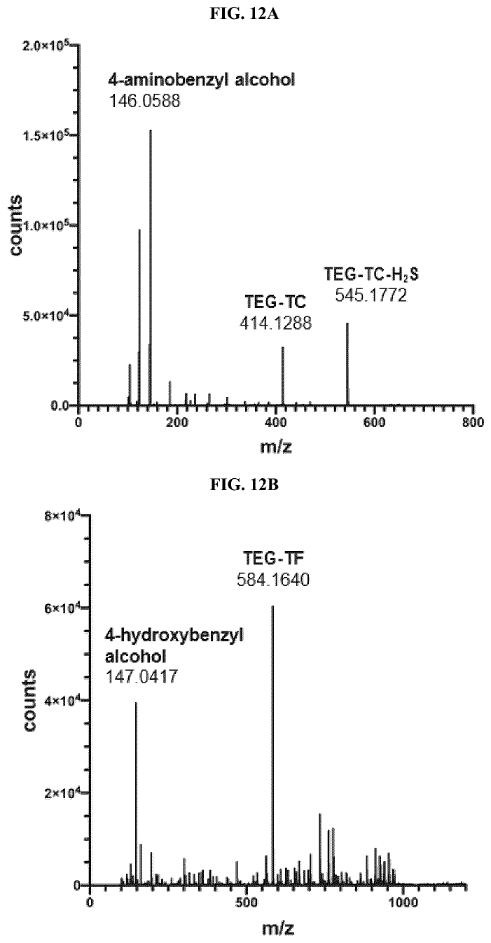

Mechanistic insight into the chemistry of the sensors was gained through investigating the reactions between the probe molecules and their cognate redox species (FIG. 5) by high-resolution mass spectrometry (HRMS) (FIGS. 12A-12B). It is worth noting that the reaction samples containing amphiphilic probes, DPPC-TC-H₂S and DPPC-TF-H₂O₂, displayed poor electrospray ionization profiles in both positive and negative ion modes. Their non-amphiphilic analogs, TEG-TC-H₂S and TEG-TF-H₂O₂, provided evidence for the formation of the uncaged products, TEG-TC and TEG-TF, upon mixing with H₂S and H₂O₂, respectively. The reaction aliquot from the TEG-TC-H₂S sample incubated with H₂S (2 hours) showed 4-aminobenzyl alcohol, suggesting a debenzylation process triggered by the reduction of the aryl azide moiety by H₂S. The TEG-TF-H₂O₂ sample incubated with H₂O₂ (1 hour) showed 4-hydroxybenzyl alcohol, suggesting a multi-step elimination cascade triggered by the oxidation of the phenylboronic pinacol ester to the benzyl alcohol, which then underwent debenzylation, followed by decarboxylation.

Next, the impact of caging and uncaging of the coumarin or fluorescein moieties on their fluorogenicity was assessed by measuring the relative fluorescence quantum yield (Φ_{Frel}) values (Table 3). Coumarin 343 was used as the standard for measuring Φ_{Frel} of the triazole-coumarin compounds (TEG-TC-H₂S, TEG-TC, DPPC-TC-H₂S, and DPPC-TC) whereas fluorescein for the triazole-fluorescein compounds (TEG-TF-H₂O₂, TEG-TF, and DPPC-TF-H₂O₂, and DPPC-TF). Relative to coumarin 343, whose absolute fluorescence quantum yield (Φ_{F}) is 0.63 in ethanol, Φ_{Frel}s of the H₂S probes TEG-TC-H₂S (entry 1) and DPPC-TC-H₂S (entry 3) were determined to be 0.07 and 0.03, respectively. Their uncaging products, TEG-TC (entry 2) and DPPC-TC (entry 4), exhibited 10-to-20-fold increase in quantum yield, with 28-fold and 120-fold enhanced brightness, respectively. The anionic form of the coumarin should exhibit a stronger fluorescence compared to its neutral analog.

The pKₐ of TEG-TC was previously found to be to be 7.1±0.1 using UV-Vis spectrophotometric measurements. Under the fluorescence activation assay conditions that are employed here (Tris 50 mM, pH 7.5), a substantial population of the coumarin moiety is expected to be in its aryloxide anion form. Relative to fluorescein, whose ΦF is 0.79 in ethanol, Φ_{Frel} values of the H₂O₂ probes TEG-TF-H₂O₂ (entry 5) and DPPC-TF-H₂O₂ (entry 7) were determined to be 0.04 and 0.02, respectively. Their uncaging products, TEG-TF (entry 6) and DPPC-TF, (entry 8) exhibited 20-to-30-fold increase in quantum yield and 49-fold or higher enhanced brightness. These results validated that caging the coumarin moiety through benzylation of its 7-O atom or the fluorescein moiety through carbonylation of its 3′ and 6′-O atoms decreases the relative fluorescence quantum yield and brightness.

TABLE 3
Characterization of exemplary ROS and SOS probes and uncaged forms thereof

| | Exₘₐₓ | Emₘₐₓ | Ext. Coeff. | Quantum | | Relative |
|---|---|---|---|---|---|---|
| Compound | (nm) | (nm) | (M⁻¹ · cm⁻¹) | Yield | Brightness | Brightness |
| TEG-TC-H₂S | 392.3 | 475.5 | 5347 | 0.07 | 374 | 5.2 |
| TEG-TC | 397.0 | 471.4 | 15613 | 0.66 | 10305 | 143.2 |
| DPPC-TC-H₂S | 396.5 | 473.5 | 24111 | 0.03 | 72 | 1.0 |
| DPPC-TC | 398.0 | 472.0 | 13749 | 0.64 | 8799 | 122.2 |
| TEG-TF-H₂O₂ | 492.0 | 526.0 | 13429 | 0.04 | 537 | 2.5 |
| TEG-TF | 494.3 | 522.6 | 35982 | 0.73 | 26267 | 122.7 |
| DPPC-TF-H₂O₂ | 495.2 | 519.5 | 10685 | 0.02 | 214 | 1.0 |
| DPPC-TF | 498.1 | 523.6 | 28990 | 0.63 | 18264 | 85.3 |

Samples were prepared using ethanol for entries 1, 2, and 4 (i.e., TEG-TC-H₂S, TEG-TC, and DPPC-TC) brightness compared to that of DPPC-TC-H₂S; for entries 5, 6, and 8 brightness compared to that of DPPC-TF-H₂O₂.

Currently, there are mixed reports on physiological concentrations of free H₂S, demonstrating that the real-time measurements of its concentration have been a long standing challenge. A physiological concentration range of 30-300 μM is considered to be normal, with levels in the central nervous system being typically higher than those in the plasma. Recently, mammalian cell responses triggered by H₂S, such as angiogenesis of endothelial cells, have been investigated using 10-600 μM exogenous H₂S.

On the basis of this study, it was reasoned that administering 500 μM Na₂S into a pH 7.5 medium (Tris, 5 mM) would generate a steady stream of H₂S at a concentration that falls within this biologically relevant concentration range. At this pH, roughly 20% of the sulfur species is expected to exist in the form of H₂S (˜100 μM), as the pKₐ of NaSH is ˜6.9. As for H₂O₂, it was sought to employ the concentration conditions under which the biogenesis of H₂O₂ has been investigated through peroxidase kinetics analyses. In accordance with these analyses, the rate of H₂O₂ production from isolated peroxisomes has been estimated to be 90 nmol/min per gram of rodent liver. Furthermore, a concentration range of 10-100 μM is characterized as an oxidative stress under physiological conditions based on protein activity assays in human alveolar adenocarcinomic cells. In light of these reports, H₂O₂ concentrations ranging from 10-100 μM were used in the investigations of TEG-TF-H₂O₂ and DPPC-TF-H₂O₂.

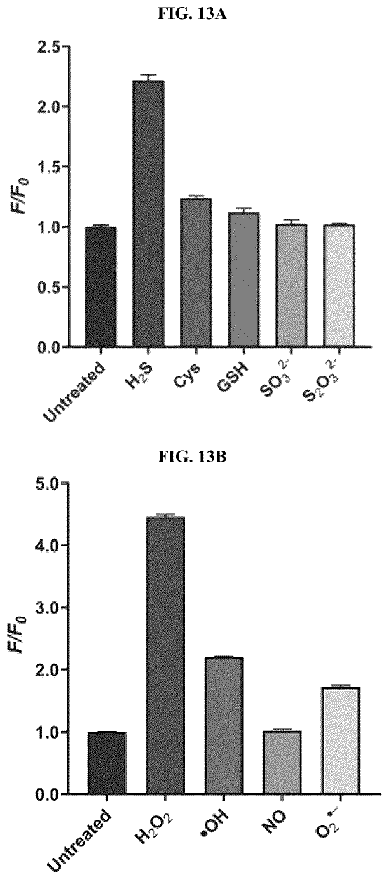

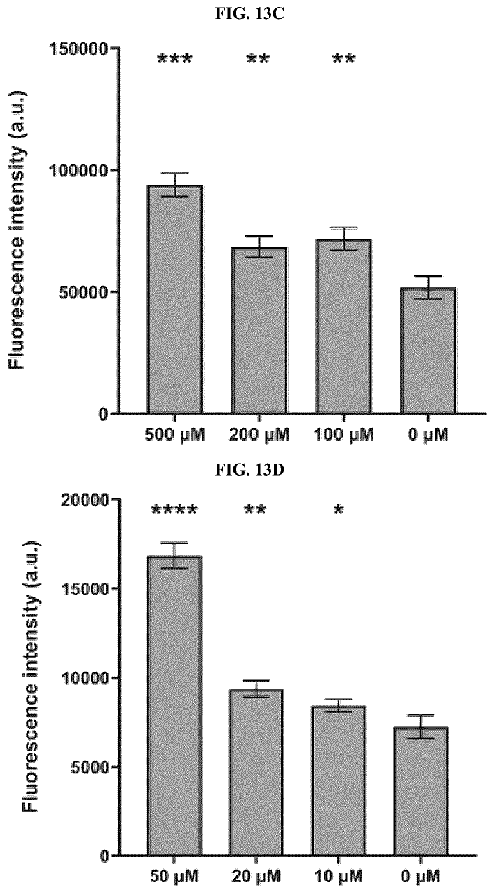

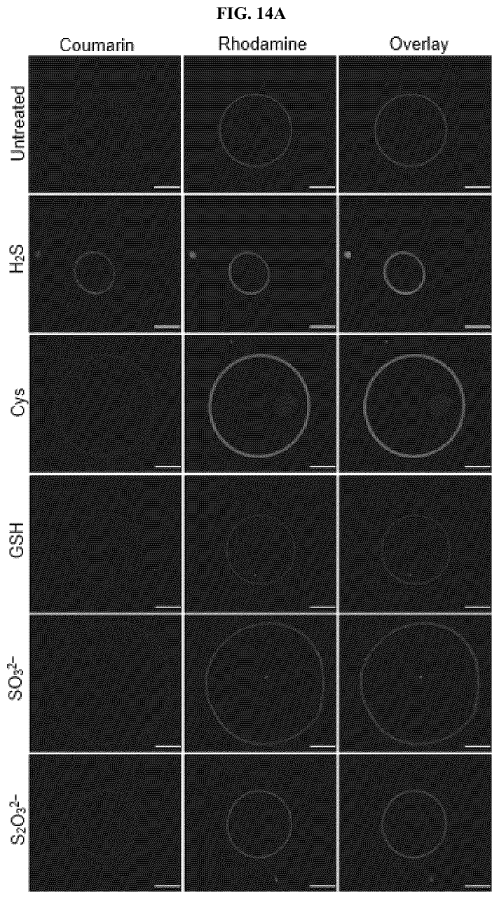

The redox-specificities of the non-amphiphilic probes, TEG-TC-H₂S or TEG-TF-H₂O₂, were assessed by subjecting them to various RSS or ROS (FIGS. 13A-13B). Change in fluorescence intensity following excitation of each sample containing the respective probe was measured over 120 minutes of incubation and presented based on the signal-to-background ratio F/F₀, where F and F₀ are defined as the fluorescence with and without the redox species. The F/F₀ of the untreated samples were set to 1 for comparisons. Upon addition of 1 mM H₂S, the sample containing TEG-TC-H₂S exhibited a steady increase in fluorescence intensity, reaching 2.2-fold fluorescence compared to the sample untreated over 2 hours of incubation (FIG. 13A). Samples containing TEG-TF-H₂O₂ and treated with 100 μM H₂O₂ presented a steady fluorescence enhancement within 2 hours, resulting in 4.4-fold higher fluorescence than those of the untreated samples (FIG. 13B). Interestingly the untreated sample showed a 277% of fluorescence increase compared to the fluorescence at 1 min, indicating that TEG-TF-H₂O₂ was possibly being hydrolyzed at pH 7.5.

The specificity of TEG-TC-H₂S toward H₂S was evaluated by comparing its reactivity against different RSS, cysteine (Cys), glutathione (GSH), sulfite (SO₃²⁻), and thiosulfate (S₂O₃²⁻). At 1 mM initial concentration and a total incubation time of 2 hours, these sulfur species induced an increase in coumarin fluorescence by less than 25% compared to the untreated sample (FIG. 13A). The measurements indicated that TEG-TC-H₂S undergoes fluorescence activation by a degree of 5-fold or more in the presence of H₂S, which induced 120% increase in fluorescence intensity, compared to the other RSS. The specificity of TEG-TF-H₂O₂ toward H₂O₂ was also assessed, by incubating the samples containing TEG-TF-H₂O₂ with 100 μM either hydroxyl radical (·OH), nitric oxide (NO), or superoxide radical (O₂·⁻) for 2 hours. Among these three reactive oxygen species, ·OH induced the highest fluorescence increase (2.2-fold), which was lower than that for H₂O₂ (4.4-fold). The relatively low yet observable reactivity of ·OH may have been due, in large part, to the residual unreacted H₂O₂ used for generating ·OH. It should be noted that the redox reaction profiles of DPPC-modified probes may differ from those observed for the TEG-modified analogs. This discrepancy may stem from the difference in availability of the probe's reactive motif to the aqueous redox milieu.

Further, the LoDs for TEG-TC-H₂S or TEG-TF-H₂O₂ were determined by evaluating the statistical differences between F and F₀ at varied concentrations of H₂S and H₂O₂, respectively, where F and F₀ are the fluorescence intensities with and without the corresponding redox active species. Here, LoD is defined as the minimum redox active species concentration at which F is statistically higher than F₀ using the one-tailed Student's t test. The LoD of TEG-TC-H₂S was determined as 100 μM (FIG. 13C), and the LoD of TEG-TF-H₂O₂ was determined as 10 μM (FIG. 13D).

### Example 3: Giant Vesicles Respond to Peroxynitrite, Hydrogen Sulfide, or Hydrogen Peroxide by Lighting Up at the Membrane

### Peroxynitrite (ONOO⁻)

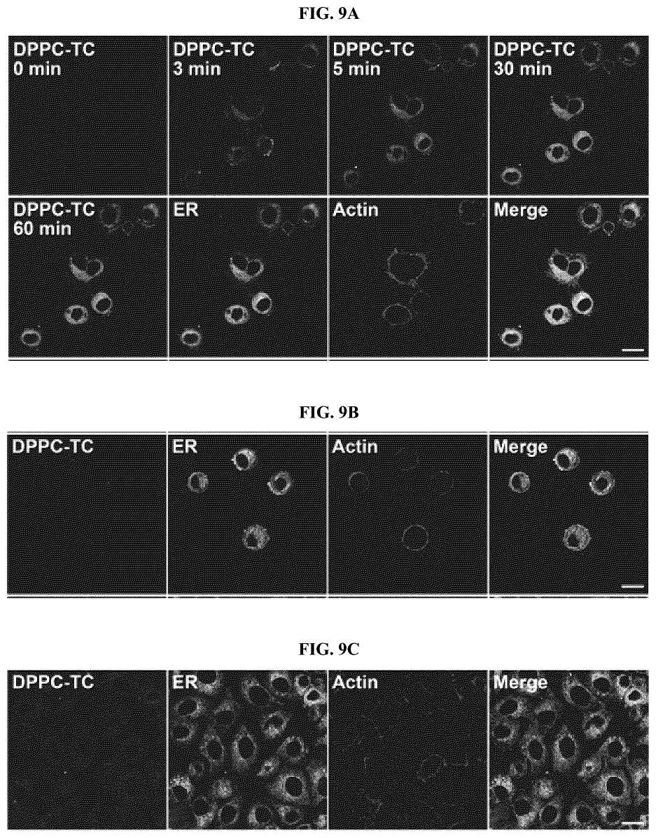

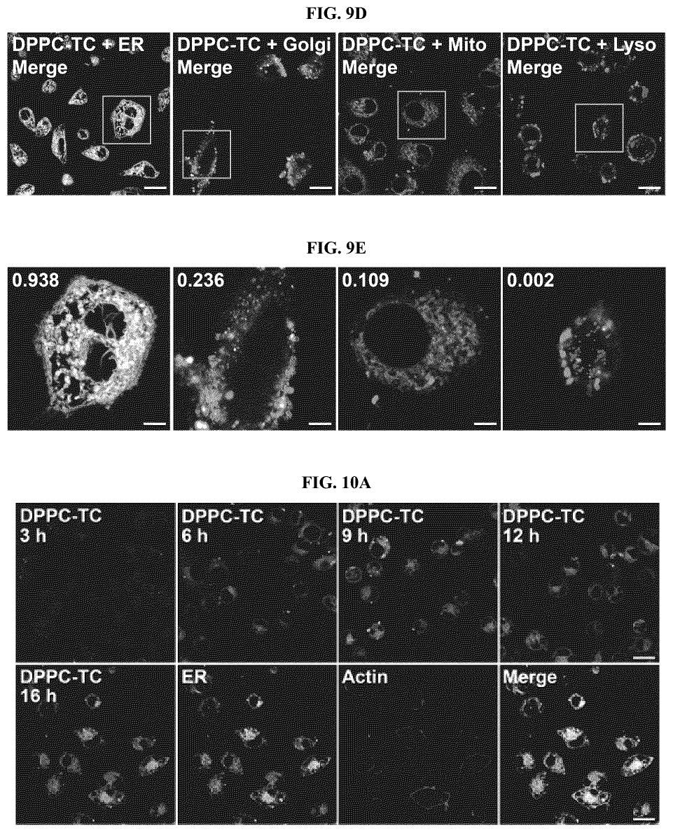

In most biomimetic systems that rely on compartmentalization, physical membrane boundaries are comprised of self-assembled lipids. Biomimetic membranes can be represented by GVs, which have dimensional properties (1-30 μm in diameter) relevant to those of mammalian cells (10-100 μm), display physical characteristics feasible for confocal fluorescence imaging, and are utilized for bottom-up construction of biomimetic systems. The membrane localization and chemical reactivity of DPPC-TC-ONOO⁻ in GVs of mixed unilamellar and multilamellar populations was studied (FIG. 9A). Given the longer timescale required for data acquisition in microscopy compared to fluorescence measurements by plate reader, the pH of imaging buffers was set to 8.5, which extended the lifetime of peroxynitrite.

GVs were produced from POPC and DPPC-TC-ONOO⁻ (˜99:1 molar ratio) using electroformation. To label vesicle membranes, 1,2-dioleoyl-sn-glycero-3-phosphoethanolamine-N-(lissamine rhodamine B sulfonyl) (18:1 Liss-Rhod PE) in 0.05 mol percent of the total lipid composition was employed. Sucrose solution (400 mM) was used to hydrate the lipid films. A high solute concentration of sucrose facilitated the formation and stabilization of GV. Following electroformation, solutions of GVs were used within 24 hours. Vesicle size distribution was analyzed by dynamic light scattering (DLS), confirming the presence of GVs. DLS analysis showed that 91% of vesicles had diameters ranging from ˜5 to 20 μm, with 48% had an average size of 5 μm in diameter. GVs were then imaged using fluorescence confocal microscopy. The vesicles were treated with the redox species, then mounted directly onto a microscope slide. Although significant vesicle motility was observed, the population density was sufficiently high to locate some static vesicles. Addition of ONOO− (100 μM final concentration) into mixture of GVs led to a substantial increase in membrane fluorescence at 405 nm excitation (FIG. 9B), which was consistent with the observations of TEG-TC-ONOO⁻ in the vesicle-free assays (FIG. 7B). Notably, DPPC-TC-ONOO⁻ makes the redox-sensing function of GVs highly selective towards ONOO−.

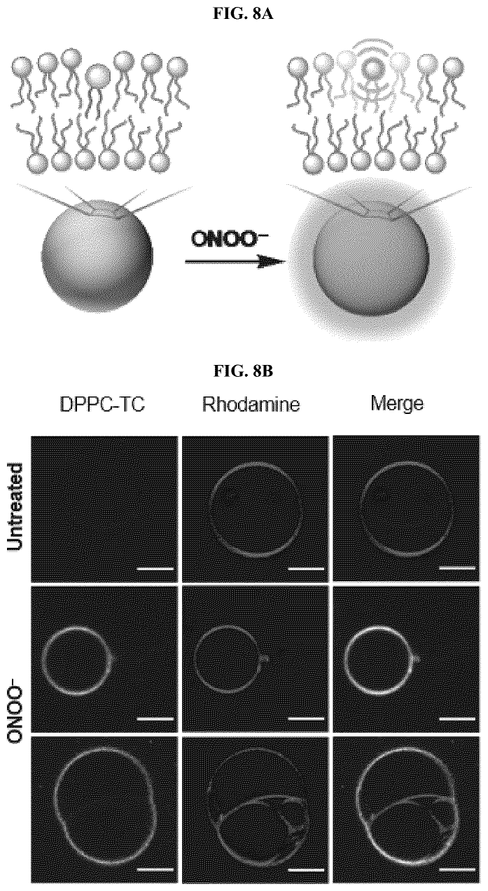

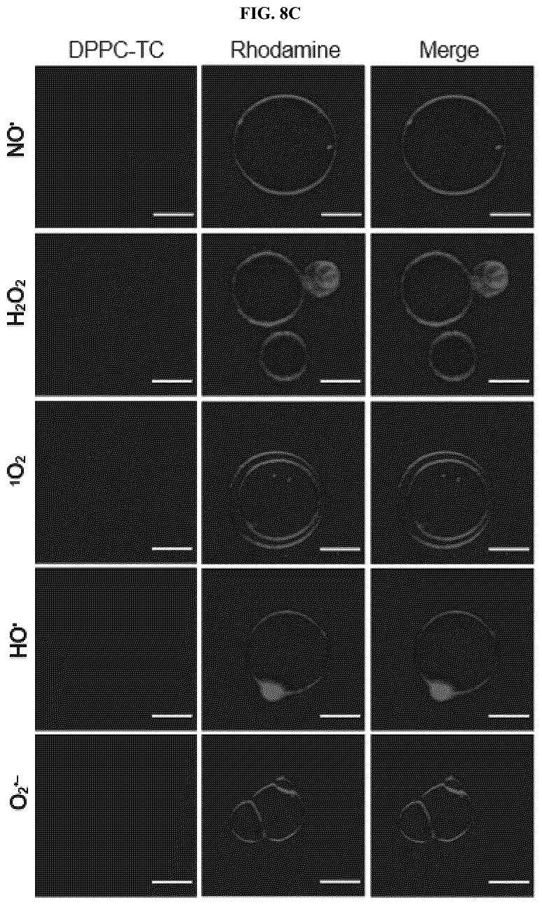

Incubation (5 min) with other redox species, including NO·, H₂O₂, HO·, ¹O₂, and O₂·⁻, resulting in in essentially no fluorescence generation following excitation at 405 nm (FIG. 8C). Taken together, these results indicate that DPPC-TC-ONOO⁻ (1) can localize into the vesicle membranes made of POPC, a natural phospholipid, (2) induces no biophysical alteration to the membrane upon being activated by ONOO−, and (3) detection of ONOO− can be achieved with high redox selectivity.

Hydrogen Sulfide (H₂S) and Hydrogen Peroxide (H₂O₂)

The redox sensing abilities of giant vesicles (GVs) comprising DPPC-TC-H₂S or DPPC-TF-H₂O₂ were investigated. In most biomimetic systems that rely on compartmentalization, physical membrane boundaries are comprised of phospholipids and cholesterol. These lipid self-assemblies have dimensional properties (1-30 μm in diameter) relevant to those of a mammalian plasma membrane (10-100 μm), display physical characteristics feasible for confocal fluorescence imaging, and are conveniently utilized for bottom-up construction of biomimetic protocells.

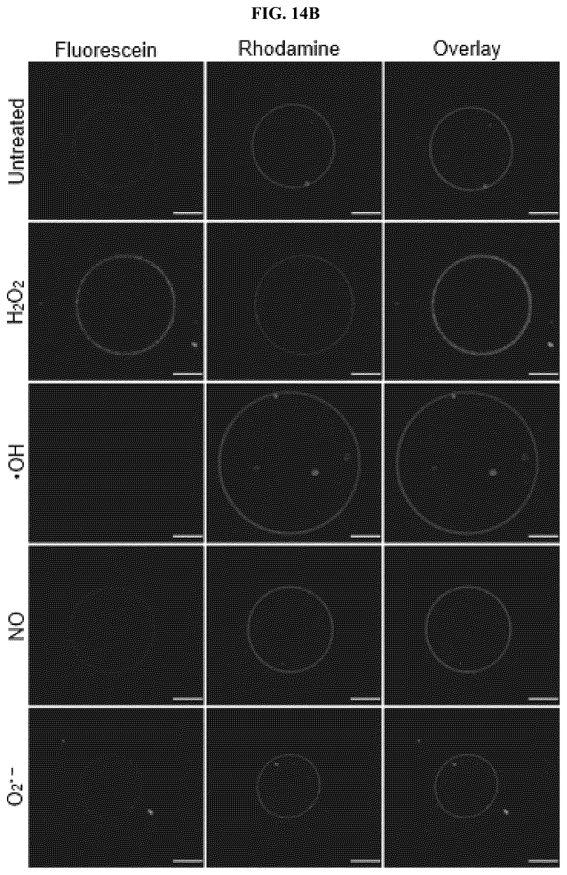

Guided by these principles, GVs (unilamellar in majority) were produced from a bulk mixture of POPC, POPG, and cholesterol (˜49:21:30 molar ratio) using electroformation and the resulting GVs were imaged by a confocal microscope (FIGS. 14A-14B). To fluorescently label the vesicle membranes, 1,2-dioleoyl-sn-glycero-3-phosphoethanolamine-N-(lissamine rhodamine B sulfonyl) (18:1 Liss-Rhod PE) at 0.1 mol % of the total lipid composition was used. A buffer solution containing sucrose (300 mM) and Tris (5 mM) at pH 7.5 was used to hydrate the lipid films. A high solute concentration of sucrose facilitated the formation and stabilization of GVs.

The vesicle size distribution was analyzed by dynamic light scattering (DLS), confirming the presence of GVs. DLS analysis showed that for the GVs containing DPPC-TF-H₂O₂, 86.4% of vesicles had diameters ranging from ˜1 to 20 μm, with 12.7% had an average size of 5 μm in diameter, while 76.2% of the GVs containing DPPC-TC-H₂S are at an average size of 2 μm in diameter. All GVs were freshly prepared prior to confocal imaging, treated with the redox species, and then mounted directly onto a microscope slide. GVs containing DPPC-TC-H₂S, POPC, POPG, and cholesterol displayed a significant increase in fluorescence upon incubation with H₂S (500 μM) over 1 hour, while incubation with equimolar of the other RSS (Cys, GSH, SO₃²⁻, and S₂O₃²⁻) reached fluorescence intensities similar to those of the untreated GVs.

These observations were conceptually in line with the assays conducted for the oxidative conditions: GVs containing DPPC-TF-H₂O₂, POPC, POPG, and cholesterol displayed a substantial fluorescence increase at the membrane after being incubated with H₂O₂ (100 PM) over 90 min. In comparison, the fluorescence intensities of those incubated with equimolar of the other ROS (NO, ·OH, and O₂·⁻) were comparable to those for the untreated GVs. Collectively, these results indicated that i) both DPPC-TC-H₂S and DPPC-TF-H₂O₂ can localize to lipid membranes made primarily of POPC, POPG, and cholesterol, ii) the resulting vesicle membrane responds to a specific redox condition, displaying high level of RSS or ROS selectivity, and iii) the fluorescently activated redox products, DPPC-TC and DPPC-TF, remain in the membrane without disrupting it.

### Example 4: DPPC-TC-ONOO Allows Direct Imaging of Intracellular Lipid Environments Targeted by Peroxynitrite

With the utility of DPPC-TC-ONOO⁻ proven in synthetic protocell membranes, incorporation into live HeLa and RAW 264.7 cells was sought, which are relevant to redox-induced stress and have well-recognized pathways for RNS and ROS production. Lipid nanoparticles (LNPs) (˜100 nm in diameter) was used to deliver DPPC-TC-ONOO− into cells. This method circumvented the limited solubility of DPPC-TC-ONOO− due to the relatively hydrophobic butanone-coumarin motif. LNPs were obtained via sonication from 1,2-di-O-octadecenyl-3-trimethylammonium propane (DOTMA), 1,2-dioleoyl-sn-glycero-3-phosphoethanolamine (DOPE), and DPPC-TC-ONOO− (47.5:47.5:5.0 molar ratio), which were hydrated with a 300 mM sucrose solution. DOTMA and DOPE were chosen as the carrier lipids due to their cationic/zwitterionic natures, which has been postulated to enhance uptake through the cell membrane.

The impact of different LNP compositions and incubation times on the cellular incorporation of DPPC-TC-ONOO⁻ was assessed. Initial trials indicated that the molar concentration of DPPC-TC-ONOO− in LNPs influenced both the homogeneity of the LNPs and the fluorescence signal generation within stimulated cells. Specifically, LNPs containing greater than 10 mol % DPPC-TC-ONOO− were considerably more heterogenous in size (up to 20 m observed, in contrast to ˜100 nm) and nonuniform in vesicle lamellarity. Those with less than 4 mol % DPPC-TC-ONOO− led to insufficient fluorescence signal. To balance these counteracting factors, 5 mol % DPPC-TC-ONOO− was used, with 47.5 mol % DOTMA and DOPE, each. In these initial assays, cells were incubated with LNPs for up to 15 hours to complete the uptake and intracellular localization of DPPC-TC-ONOO−. Shorter incubation times (3-to-5 hours) led to its uptake but not subcellular localization. To facilitate extended incubation timeframes, LNPs were prepared in DMEM (Dulbecco's Modified Eagle Medium) instead of HBSS (Hanks' Balanced Salt Solution), which was observed to be deleterious to the cells for incubations exceeding 3 hours. However, high serum concentration in DMEM caused interference between LNPs and serum-protein, resulting in aggregate formation as observed by microscopy. To overcome this obstacle, LNPs (50 μM) were used in a mixture of 2/3 volumetric ratio of sucrose solution and Opti-MEM™, a reduced serum medium known to increase the efficiency of lipofection.

With a feasible liposome preparation and treatment protocol established for live cell imaging, attention was turned to assessing the cytocompatibility of the LNPs, with or without DPPC-TC-ONOO⁻, DPPC-TC, and the oxa-spiro[4,5] decenone 6a. A standard 3-(4,5-dimethylthiazol-2-yl)-2,5-diphenyl tetrazolium bromide (MTT) cell viability assay in HeLa and RAW 264.7 cells treated with these molecules (8-250 μM) over a 24-hour period. The dose-response curves provided high IC50 values for each compound: DPPC-TC-ONOO− (≥100 μM), DPPC-TC (≥50 μM), and 6 (≥70 μM). These results suggested that the probe-containing LNPs (50 μM) and the byproducts of the reaction from peroxynitrite (DPPC-TC and 6) do not induce cytotoxicity under the cell imaging conditions used.

The biological utility of DPPC-TC-ONOO⁻ was first demonstrated by imaging live HeLa cells that are under oxidative and nitrosative stress (FIGS. 9A-9E). For a physiologically relevant stress model, the standard stimulation condition involving IFN-γ/LPS/PMA was employed. Treatment of cells with IFN-γ/LPS leads to the endogenous production of nitric oxide and PMA provokes increased levels of superoxide. These two reactive species combine to form peroxynitrite, as described elsewhere herein. HeLa cells treated with LNPs containing DPPC-TC (positive control) exhibited strong coumarin fluorescence at 405/475 nm. In contrast, those treated with LNPs containing DPPC-TC-ONOO⁻ and left unstimulated (negative control) generated no observable signal.

Upon endogenous peroxynitrite generation through stimulation with IFN-γ/LPS/PMA, a substantial increase in fluorescence at 405/475 nm was observed (FIG. 9A). Notably, upon PMA addition post IFN-γ/LPS stimulation, the intracellular fluorescence was observable at the 3-minute time point, the earliest possible image acquisition time after cell treatment in this imaging setup. This indicated that, in the background of stimulated iNOS function, PMA instantly leads to ONOO− production (presumably by activating NADPH oxidase directly) and that DPPC-TC-ONOO− can rapidly sense proximal ONOO−. The change in signal intensity after 30 minutes was negligible, suggesting the possibility of probe consumption and/or lack of proximal peroxynitrite.

The intracellular redox selectivity of DPPC-TC-ONOO⁻ was also assessed using N-(3-(aminomethyl)benzyl)-acetamidine, known as 1400 W, which inhibits the production of NO· by irreversibly binding to iNOS. The cells treated with 1400 W only (FIG. 9B) or treated with 1400 W and IFN-γ/LPS/PMA (FIG. 9C) generated no observable signal in the DPPC-TC channel. These controls showed that DPPC-TC-ONOO⁻ selectively senses peroxynitrite generated from iNOS in response to nitrosative but not oxidative stress.

To determine whether DPPC-TC-ONOO⁻ localizes and functions at a specific subcellular compartment in HeLa, a comprehensive colocalization study was performed with dyes specific for lipid-rich and membrane-enclosed organelles, including the ER, mitochondrion, Golgi apparatus, and lysosome (FIGS. 9D-9E). In addition, actin stain was used to gain a visual insight into the cytoskeletal structure of the whole cell.

To assess the degree of colocalization quantitatively, the Pearson's correlation coefficients (PCC) of the signal from DPPC-TC overlapping with that from the organelle tracker dyes were used. The DPPC-TC signal overlapped almost perfectly (PCC=0.938) with the perinuclear signal of ER-Tracker™ Green, suggesting a high statistical correlation and preference of DPPC-TC-ONOO− to localize in the ER. Cells stained with dyes specific for Golgi, mitochondria, and lysosomes displayed less significant correlation.

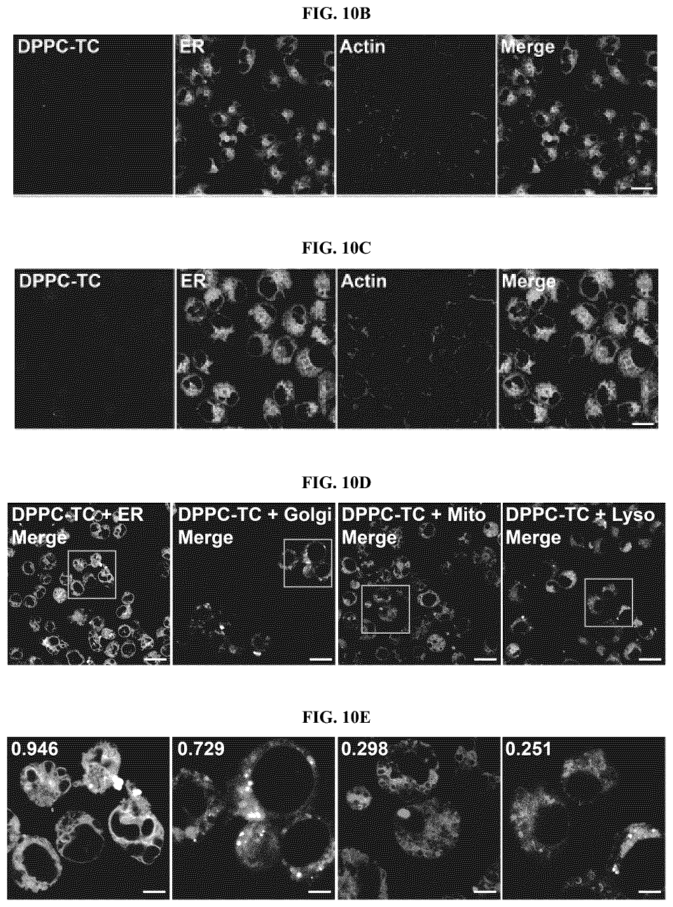

The investigations were expanded to include murine-derived macrophage model, RAW-264.7 (FIGS. 10A-10E). RNS play important roles in macrophage activation and differentiation as part of the inflammatory response. Treatment of RAW-264.7 cells with LPS leads to the expression of iNOS, which produces NO·, which then reacts with O₂·⁻, generating ONOO−. RAW-264.7 cells were treated with LNPs containing either DPPC-TC (positive control) or DPPC-TC-ONOO⁻ (FIGS. 10A-10E). Those treated with LNPs containing DPPC-TC-ONOO⁻ and left unstimulated generated negligible levels of signal at 405/475 nm (“DPPC-TC channel”). Endogenous peroxynitrite production through stimulation with LPS led to a substantial increase in fluorescence intensity over 16 hours (FIG. 10A). The intracellular redox selectivity of DPPC-TC-ONOO⁻ was evaluated using 1400 W. The cells treated with 1400 W only (FIG. 10B) or treated with 1400 W and LPS (FIG. 10C) generated little-to-no signal in the DPPC-TC channel. Assessment of DPPC-TC-ONOO⁻ localization in RAW-264.7 cells was carried out using the ER, Golgi, mitochondrion, and lysosome trackers (FIGS. 10D-10E). The PCC values for the merged images of ER (0.946) and Golgi (0.729) suggested that DPPC-TC-ONOO⁻ localizes primarily in the ER and then Golgi. This observation suggests that the probe localization is not exclusive for the ER, likely due to the abundance of ER-Golgi intermediate compartments (ERGIC) in RAW-264.7 cells and increased prevalence in cross talk between the ER and Golgi.

In contrast to the observations with DPPC-TC-ONOO−, its non-amphiphilic counterpart, TEG-TC-ONOO−, displayed inferior specificity in regard to subcellular localization. It is worth noting that the use of LNPs proved unsuccessful for cellular uptake of either TEG-TC or TEG-TC-ONOO−. Therefore, cells were treated with TEG-TC (positive control) or TEG-TC-ONOO− directly and incubated for 20 minutes. Those treated with TEG-TC-ONOO− were then stimulated via the methods described above. Both the positive controls and stimulated cells displayed DPPC-TC signal throughout the cells, including the nucleus. In the case of RAW 264.7 cells, the intracellular homogeneity of the signal was especially substantial.

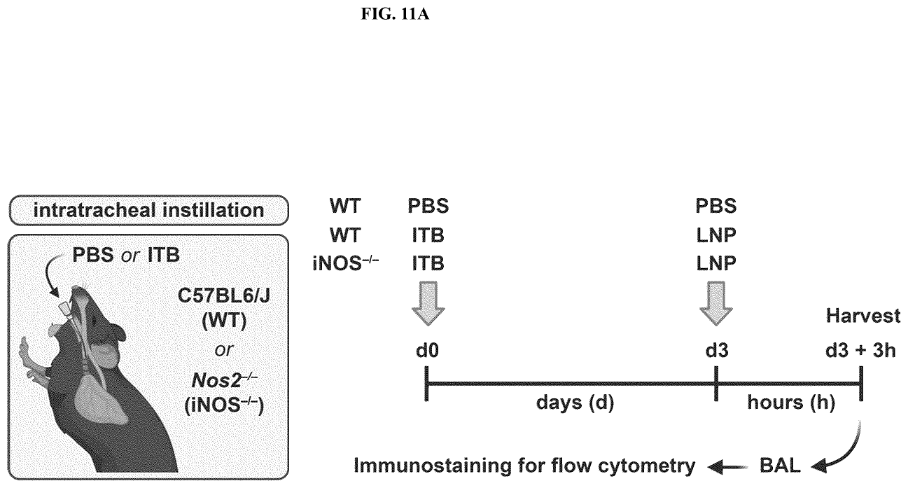

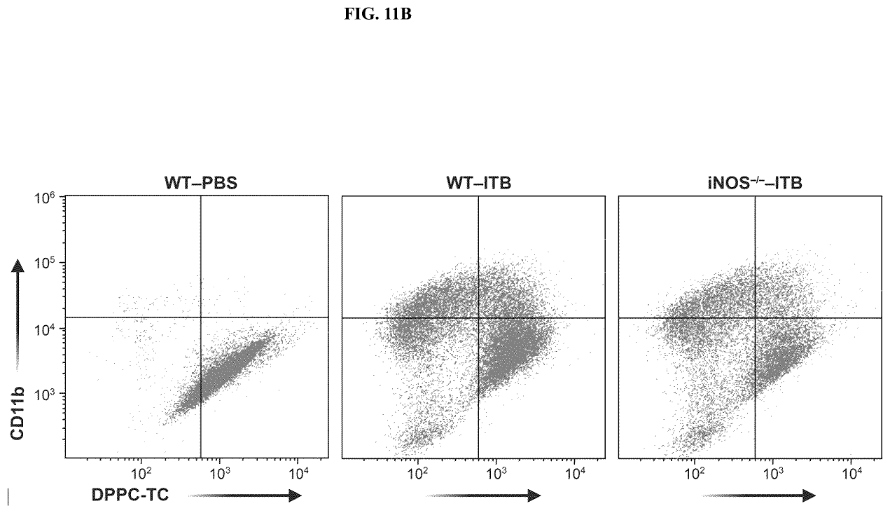

After successfully imaging the iNOS dependent peroxynitrite activity in both HeLa and RAW264.7, the potential of DPPC-TC-ONOO⁻ for detecting peroxynitrite generated in lung tissue injury using a pre-established murine model of ALI was explored (FIGS. 11A-11B). During ALI, release of cytokines and chemokines, such as tumor necrosis factor-alpha (TNF-α) and interleukin-1 beta (IL-10), recruit macrophages and other immune cells to the site of injury. During the initial phase of injury (d0-d3), pro-inflammatory macrophages are dominant, which is associated with higher levels of iNOS expression.

Herein, a previously reported ITB method was employed, which causes a severe inflammatory response characterized by infiltration of immune cells, which is dependent upon iNOS activation. For a generalizable and biocompatible instillation protocol, LNP concentrations ranging from 0.5 to 2.0 mM were evaluated.

LNPs carrying DPPC-TC (5 mol % of the lipid content) were employed in C57BL6/J mice and the viability of the cells from BAL was analyzed via flow cytometry. Results indicated 94% viability at 0.5 mM LNP concentration, which was employed for the subsequent experiments. On d0, mice (C57BL6/J or iNOS^{-/-}) were anesthetized and received an intratracheal instillation of either PBS (control) or bleomycin (FIGS. 11A-11B). To detect the peroxynitrite generation during the early inflammatory response, mice were again anesthetized and instilled with 0.5 mM LNPs containing DPPC-TC-ONOO⁻ (5 mol % of the lipid content) on day 3. The animals were sacrificed, and BAL was collected for immunostaining 3 hour-post instillation.

Flow staining was used to define myeloid derived cells (CD45+), migratory (CD11b+) and pulmonary in nature (CD11c⁺).

A significant increase in recruited population as a result of ITB was observed for both WT (13±2.4%) and iNOS−/−(17±6.1%) compared to healthy WT (2±0.8%). This increase was also observed for the migratory, and hence activated, population due to ITB in WT (28±6.6%) and iNOS−/−(20±8.2%), compared to control WT (2±0.5%) (FIG. S14). The migratory population was further analyzed for DPPC-TC signal quantification (Table 4). As indicated by the increase in migratory macrophages in both iNOS−/− and C57Bl6/J mice following ITB, inflammatory activation was not altered significantly by the lack of NO· generation. However, the degree of DPPC-TC fluorescence generated in response to ITB in iNOS−/− (entry 3) was no different from control (entry 1), while it was significantly increased in C57BL6/J (entry 2). These data indicate that DPPC-TC-ONOO⁻ was successfully delivered to the BAL cells in vivo and was able to quantify the production of ONOO⁻ with ALI.

TABLE 4
Characterization of the DPPC-TC positive-to-negative
(p:n) ratio in CD11b⁺ macrophage population
from BAL cells (n = 3 per group)

| Entry | Treatment | DPPC-TC p:n ratio |
|---|---|---|
| 1 | WT-PBS | 0.55 ± 0.021 |
| 2 | WT-ITB | 0.72 ± 0.008 |
| 3 | iNOS^{-/-}-ITB | 0.56 ± 0.018 |

### ENUMERATED EMBODIMENTS

The following exemplary embodiments are provided, the numbering of which is not to be construed as designating levels of importance:

Embodiment 1 provides a compound selected from the group consisting of:

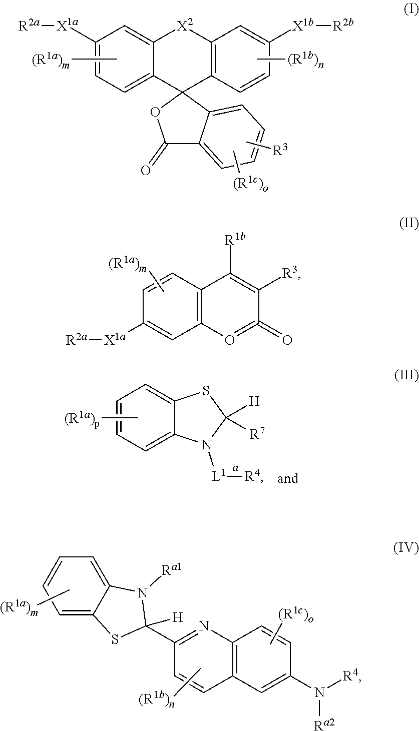

wherein:
R^{1a}, R^{1b}, R^{1c}, R^{1d}, R^{1e}, R^{1f}, and R^{1g}, if present, are each independently selected from the group consisting of optionally substituted C₁-C₆ alkyl, optionally substituted C₃-C₈ cycloalkyl, optionally substituted C₂-C₈ heterocycloalkyl, optionally substituted C₆-C₁₀ aryl, optionally substituted C₂-C₁₀ heteroaryl, halogen, CN, NO₂, OR^{A}, N(R^{A})(R^{B}), C(═O)OR^{A}, C(═O)N(R^{A})(R^{B}), S(═O)₂N(R^{A})(R^{B}), S(═O)N(R^{A})(R^{B}), OC(═O)R^{A}, and N(R^{A})C(═O)R^{B};
R^{2a} and R^{2b}, if present, are each independently selected from the group consisting of:

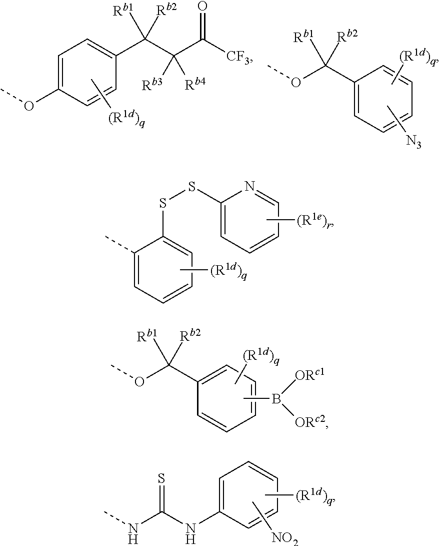

B(OR^{c1})(OR^{c2}), N(R^{A})(R^{B}), and OR^{A},
   wherein no more than one of R^{2a} and R^{2b} is N(R^{A})(R^{B}) or OR^{A};
R³, if present, is

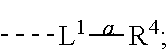

R⁴ is selected from the group consisting of optionally substituted C₁-C₃₀ alkyl, optionally substituted C₂-C₃₀ heteroalkyl, optionally substituted C₂-C₃₀ alkenyl, optionally substituted C₂-C₃₀ alkynyl,

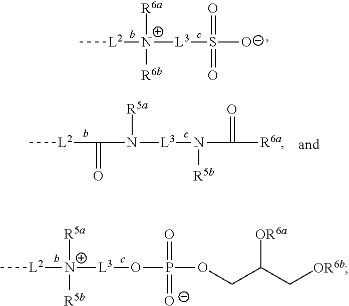

R^{5a} and R^{5b}, if present, are each independently selected from the group consisting of H, optionally substituted C₁-C₆ alkyl, optionally substituted C₃-C₈ cycloalkyl, optionally substituted C₆-C₁₀ aryl, and optionally substituted C₂-C₁₀ heteroaryl;
R^{6a} and R^{6b}, if present, are each independently selected from the group consisting of optionally substituted C₁-C₃₀ alkyl, optionally substituted C₂-C₃₀ heteroalkyl, optionally substituted C₂-C₃₀ alkenyl, optionally substituted C₂-C₃₀ alkynyl, C(═O)(optionally substituted C₁-C₃₀ alkyl), C(═O)(optionally substituted C₂-C₃₀ heteroalkyl), C(═O)(optionally substituted C₂-C₃₀ alkenyl), and C(═O)(optionally substituted C₂-C₃₀ alkynyl);
R⁷, if present, is

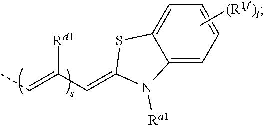

L¹, if present, is selected from the group consisting of:

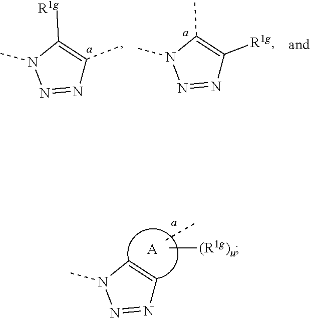

ring A, if present, is selected from the group consisting of C₅-C₁₂ cycloalkyl and C₂-C₁₀ heterocycloalkyl;
L² and L³ are each independently selected from the group consisting of optionally substituted C₁-C₁₂ alkylenyl, optionally substituted C₂-C₁₂ heteroalkylenyl, optionally substituted C₃-C₁₂ cycloalkylenyl, optionally substituted C₂-C₁₀ heterocycloalkylenyl, optionally substituted C₆-C₁₀ arylenyl, and optionally substituted C₂-C₁₂ heteroarylenyl;
X^{1a} and X^{1b}, if present, are each independently selected from the group consisting of a bond, —C(═O), —OC(═O)—, and —N(R^{A})C(═O)—;
X², if present, is selected from the group consisting of O and Si(R^{A})(R^{B});
R^{a1} and R^{a2} if present, are each independently selected from the group consisting of H, optionally substituted C₁-C₆ alkyl, C(═O)(optionally substituted C₁-C₆ alkyl), and hydrocarbyl; R^{b1}, R^{b2}, R^{b3}, and R^{b4}, if present, are each independently selected from the group consisting of H and optionally substituted C₁-C₆ alkyl;
R^{c1} and R^{c2}, if present, are each independently selected from the group consisting of H and optionally substituted C₁-C₆ alkyl, or
   R^{c1} and R^{c2} combine with the atoms to which they are bound to form an optionally substituted C₂-C₈ heterocycloalkyl;
each occurrence of R^{d1}, if present, is independently selected from the group consisting of H and optionally substituted C₁-C₆ alkyl,
   wherein two R^{d1} groups having a 1,3-relationship can combine with the atoms to which they are bound to form an optionally substituted C₅-C₁₀ cycloalkenyl;
R^{A} and R^{B} are each independently selected from the group consisting of H, optionally substituted C₁-C₆ alkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₆-C₁₀ aryl, optionally substituted C₂-C₁₀ heteroaryl;
m, n, and o are each independently selected from the group consisting of 0, 1, 2, and 3;
p, q, r, and t are each independently selected from the group consisting of 0, 1, 2, 3, and 4
s is selected from the group consisting of 1, 2, 3, and 4; and
u is selected from the group consisting of 0, 1, 2, 3, 4, 5, 6, 7, 8, 9, and 10;
or a salt, solvate, stereoisomer, or isotopologue thereof.

Embodiment 2 provides the compound of Embodiment 1, wherein R⁴ is selected from the group consisting of

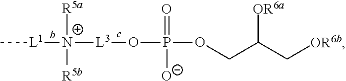

—(CH₂)₁₋₁₀C(═O)NH(CH₂)₂₋₁₀NHC(═O)(CH₂)₀₋₉CH₃, —(CH₂)₁₋₁₀N⁺[(CH₂)₀₋₉CH₃]₂(CH₂)₁₋₁₀S(═O)₂O⁻ and

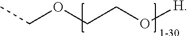

Embodiment 3 provides the compound of Embodiment 1 or 2, wherein R⁴ is selected from the group consisting of:

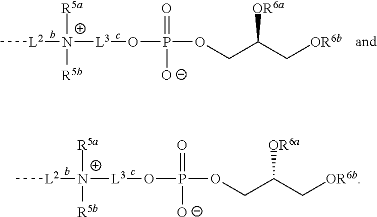

Embodiment 4 provides the compound of any one of Embodiments 1-3, wherein L² and L are each independently selected from the group consisting of:
—CH₂—, —(CH₂)₂—,

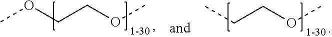

Embodiment 5 provides the compound of any one of Embodiments 1-4, wherein R^{5a} and R^{5b} are each independently CH₃.

Embodiment 6 provides the compound of any one of Embodiments 1-5, wherein R^{6a} and R^{6b} are each independently C(═O)(C₁₀-C₂₀ alkyl), optionally wherein R^{6a} and R^{6b} are each independently C(═O)(pentadecanyl).

Embodiment 7 provides the compound of any one of Embodiments 1-6, wherein R⁴ is:

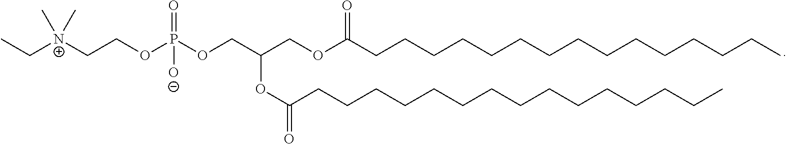 [SMILES: CCCCCCCCCCCCCCCC(=O)OCC(COP(=O)([O-])OCC[N+](C)(C)CC)OC(=O)CCCCCCCCCCCCCCC]

Embodiment 8 provides the compound of any one of Embodiments 1-7, wherein L¹ is selected from the group consisting of:

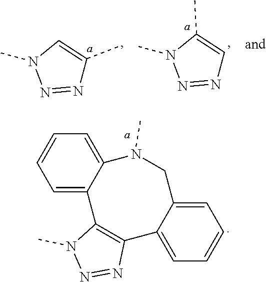

Embodiment 9 provides the compound of any one of Embodiments 1-8, wherein at least one of R^{2a} and R^{2b} is selected from the group consisting of:
B(OH)₂,

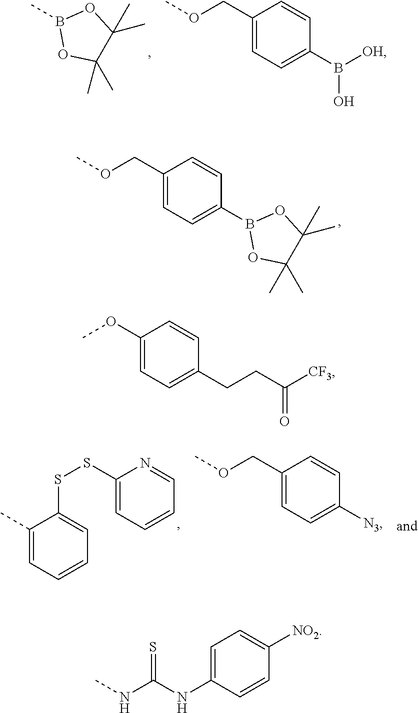

Embodiment 10 provides the compound of any one of Embodiments 1-9, wherein the compound is selected from the group consisting of:

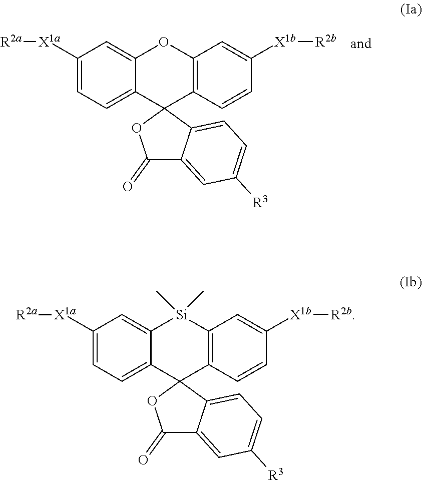

Embodiment 11 provides the compound of Embodiment 10, wherein one of the following applies:
(a) X^{1a} and X^{2a} combine to form

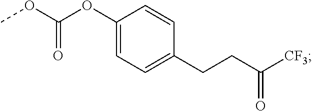

and X^{1b} and X^{2b} combine to form

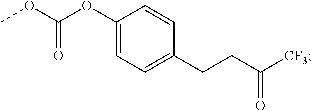

(b) X^{1a} and X^{2a} combine to form

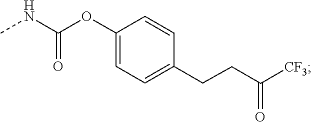

and X^{1b} and X^{2b} combine to form

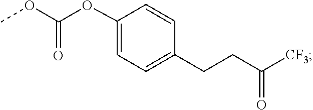

(c) X^{1a} and X^{2a} combine to form

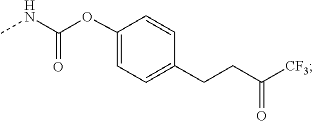

and X^{1b} and X^{2b} combine to form

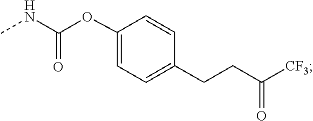

(d) X^{1a} and X^{2a} combine to form

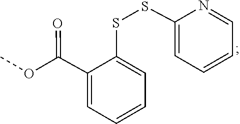

and X^{1b} and X^{2b} combine to form

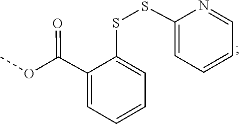

(e) X^{1a} and X² combine to form

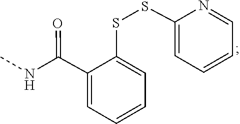

and X^{1b} and X^{2b} combine to form

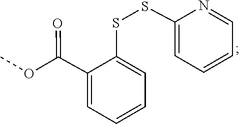

(f) X^{1a} and X^{2a} combine to form

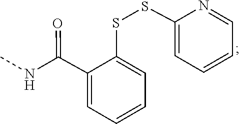

and X^{1b} and X^{2b} combine to form

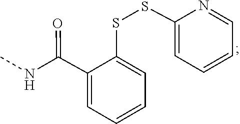

(g) X^{1a} and X^{2a} combine to form

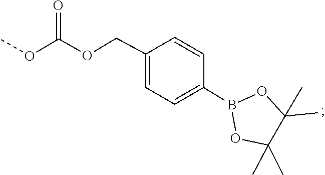

and X^{1b} and X^{2b} combine to form

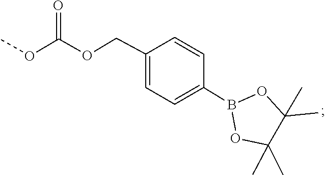

(h) X^{1a} and X^{2a} combine to form

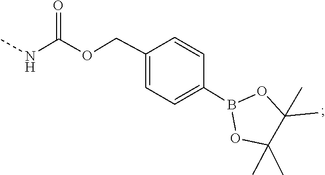

and X^{1b} and X^{2b} combine to form

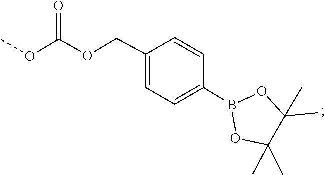

and
(i) X^{1a} and X^{2a} combine to form

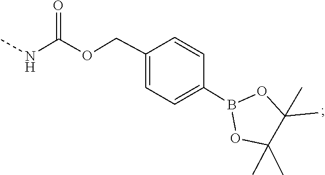

and X^{1b} and X^{2b} combine to form

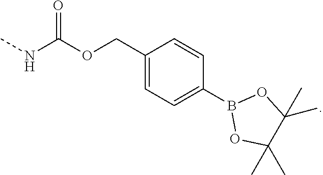

Embodiment 12 provides the compound of any one of Embodiments 1-9, wherein the compound is:

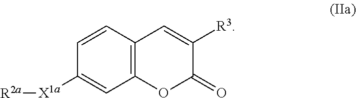

Embodiment 13 provides the compound of Embodiment 12, wherein one of the following applies:
(a) X^{1a} and X^{2a} combine to form

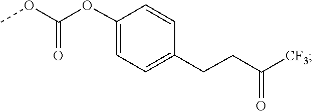

(b) X^{1a} and X^{2a} combine to form

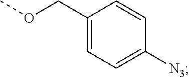

(c) X^{1a} and X^{2a} combine to form

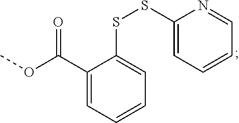

and
(d) X^{1a} and X^{2a} combine to form

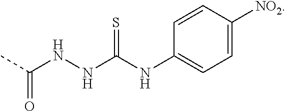

Embodiment 14 provides the compound of any one of Embodiments 1-7, wherein the compound is:

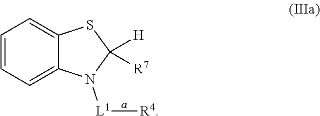

Embodiment 15 provides the compound of Embodiment 14, wherein L¹ is —(CH₂)₂—.

Embodiment 16 provides the compound of Embodiment 14 or 15, wherein R⁷ is selected from the group consisting of:

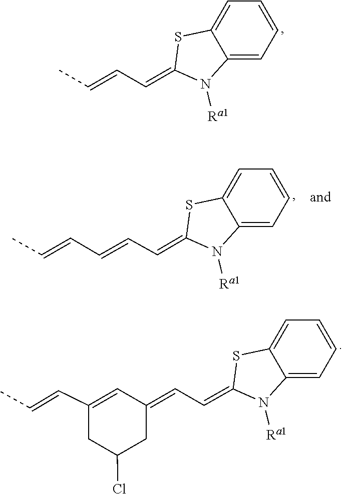

Embodiment 17 provides the compound of Embodiment 16, wherein R^{a1} is selected from the group consisting of H and CH₃.

Embodiment 18 provides the compound of any one of Embodiments 1-7, wherein the compound is:

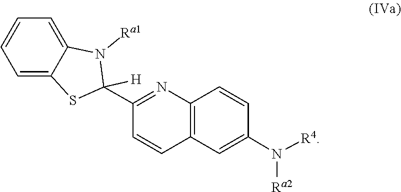

Embodiment 19 provides the compound of Embodiment 18, wherein R^{a1} and R^{a2} are each independently selected from the group consisting of H and CH₃.

Embodiment 20 provides the compound of any one of Embodiments 1-19, which is selected from the group consisting of:

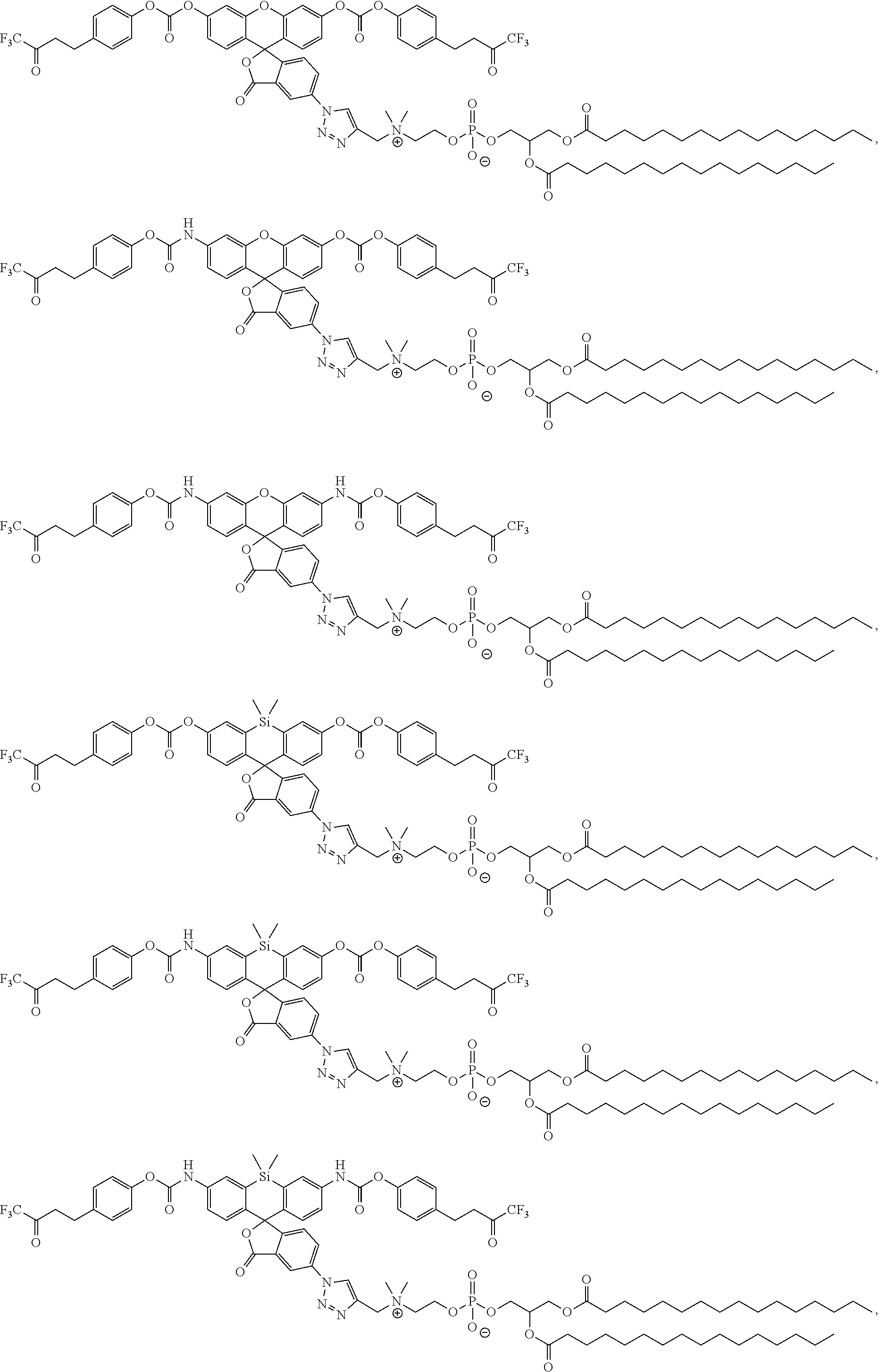

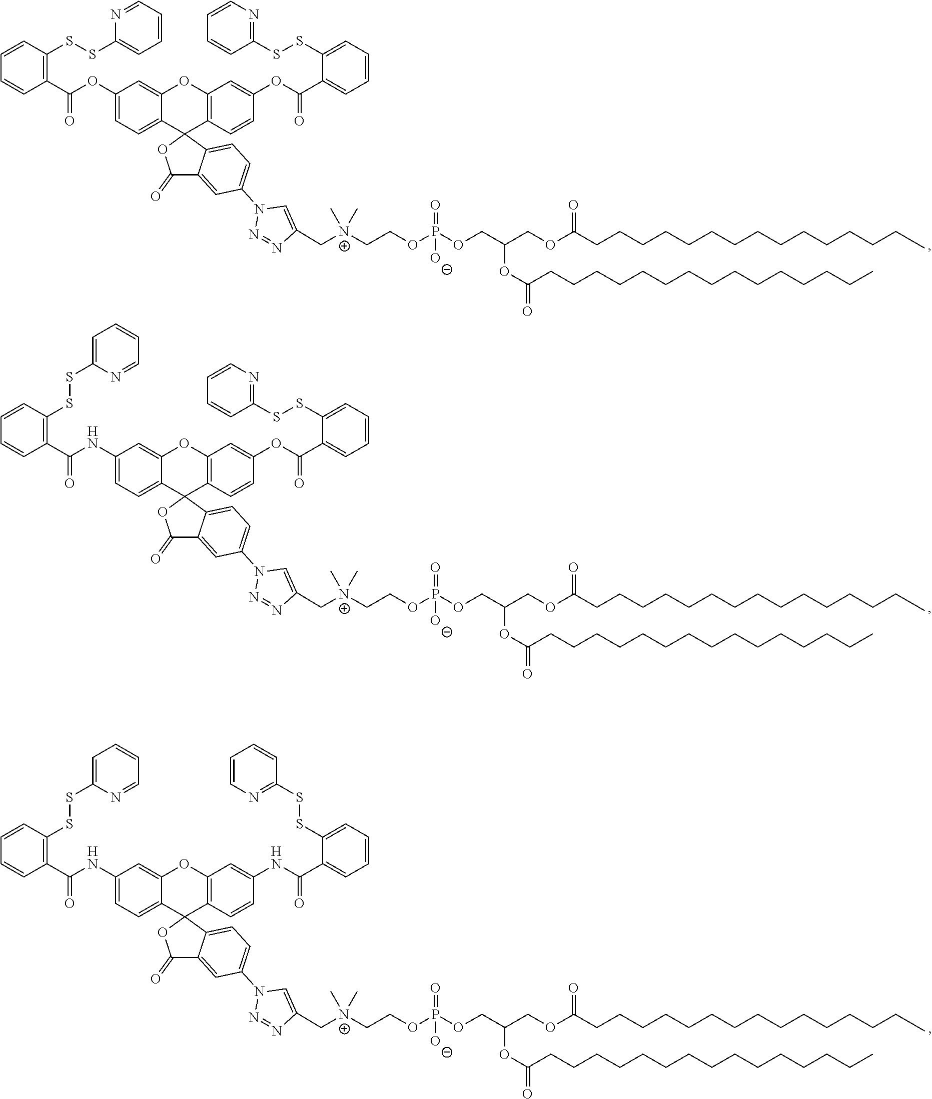

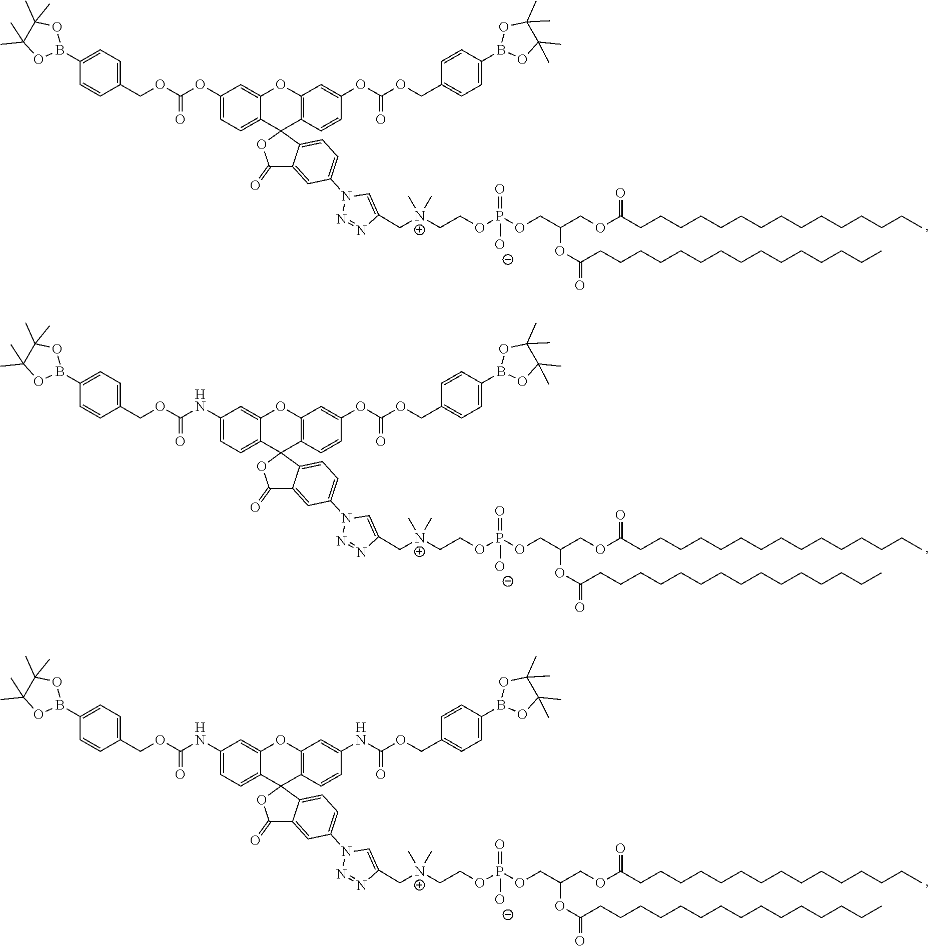

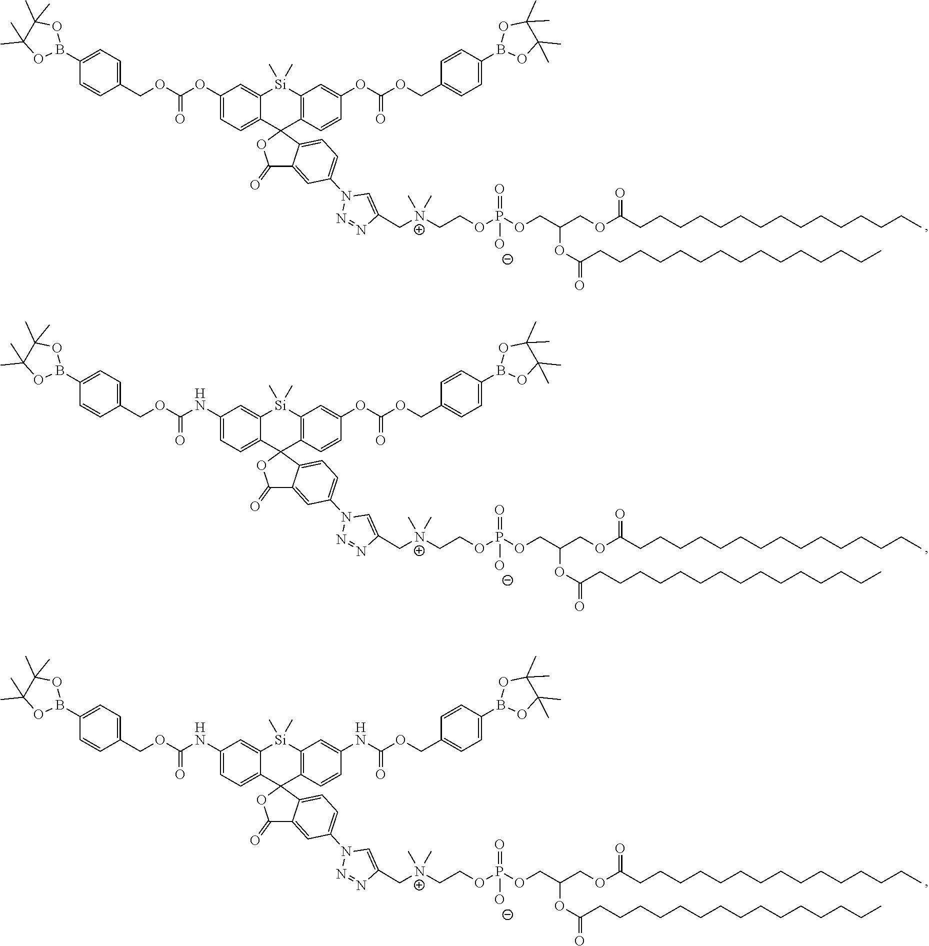

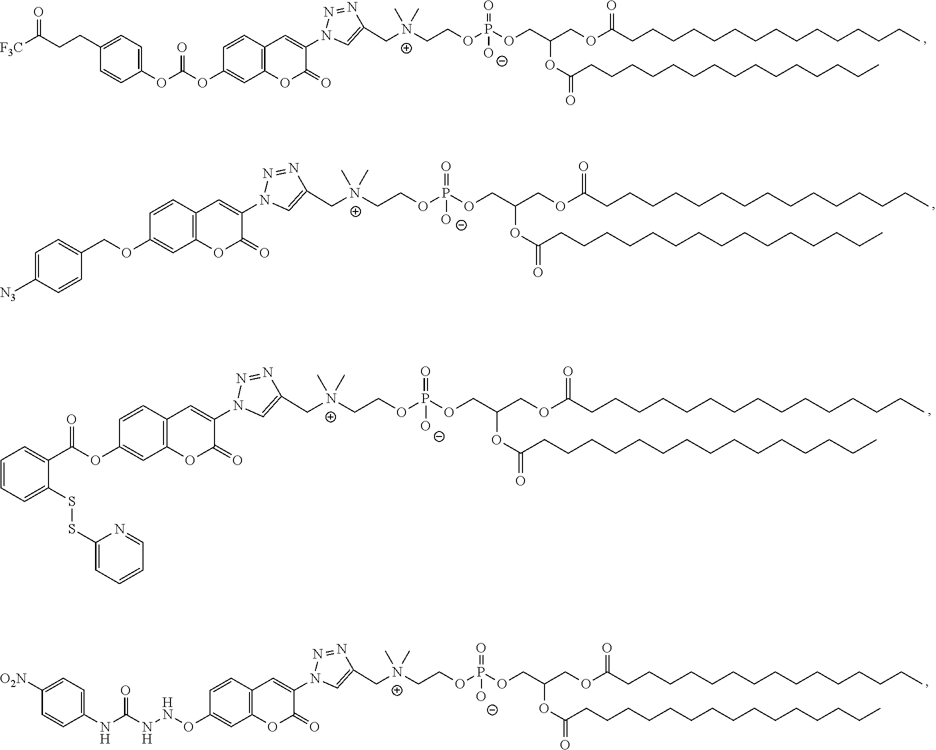

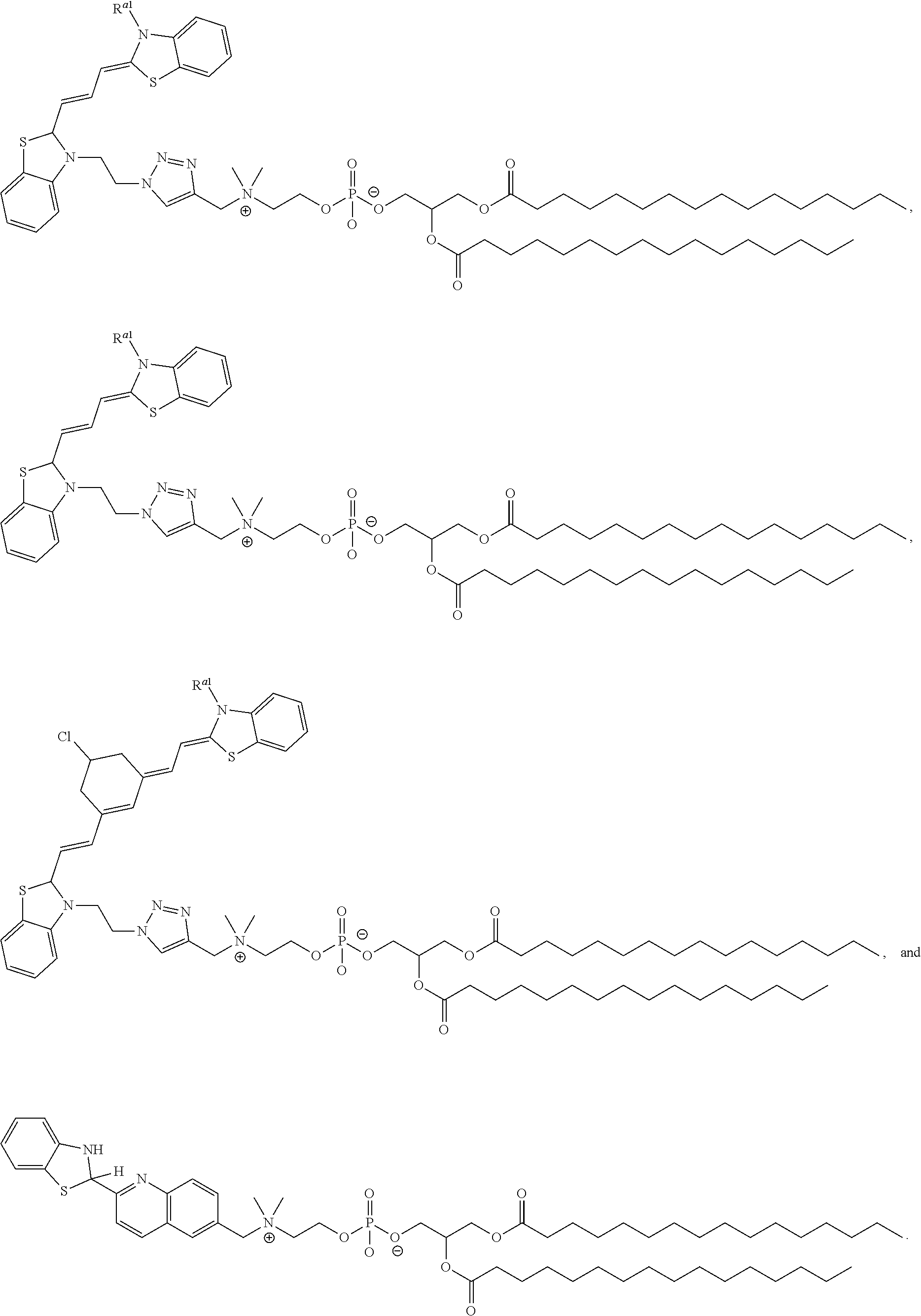

Embodiment 21 provides a lipid particle comprising:
(a) at least one compound of any one of Embodiments 1-20; and
(b) at least one additional lipid.

Embodiment 22 provides the lipid particle of Embodiment 21, wherein (a) and (b) have a molar ratio ranging from about of about 1:99 to about 10:90.

Embodiment 23 provides the lipid particle of Embodiment 21 or 22, wherein the at least one additional lipid comprises at least one selected from the group consisting of a phospholipid and cholesterol, or a modified derivative thereof.

Embodiment 24 provides the lipid particle of any one of Embodiments 21-23, wherein the at least one additional lipid comprises at least one selected from the group consisting of POPC, 1-hexadecanoyl-2-(9Z-octadecenoyl)-sn-glycero-3-phosphoglycerol (POPG), DOTMA, DOPE, and cholesterol.

Embodiment 25 provides a method of detecting a redox biomarker in a sample, the method comprising:
(a) contacting the compound of any of Formulae (I)—(IV) of any one of Embodiments 1-20 and/or the lipid particle of any one of Embodiments 21-24 with the sample to provide a mixture; and
(b) detecting a fluorescent signal in the mixture, wherein the fluorescent signal is indicative of a redox biomarker in the sample.

Embodiment 26 provides the method of Embodiment 25, wherein the sample is a biological sample obtained from a subject.

Embodiment 27 provides the method of Embodiment 25 or 26, wherein the redox biomarker is selected from the group consisting of peroxynitrite (ONOO⁻), hydrogen sulfide (H₂S), hydrogen peroxide (H₂O₂), superoxide (O₂·⁻), and nitric oxide (NO·).

Embodiment 28 provides the method of any one of Embodiments 25-27, wherein the contacting of the compound any of Formulae (I)—(IV) with the redox biomarker in the sample results in chemical modification of the compound of any of Formulae (I)—(IV).

Embodiment 29 provides a method of diagnosing a disease or disorder associated with oxidative, nitrosative, or nitrative stress in a subject, the method comprising:
(a) contacting the compound of any of Formulae (I)—(IV) of any one of Embodiments 1-20 and/or the lipid particle of any one of Embodiments 21-24 with a sample obtained from the subject to provide a mixture; and
(b) detecting a fluorescent signal in the mixture, wherein the fluorescent signal is indicative of a disease or disorder associated with oxidative, nitrosative, or nitrative stress.

Embodiment 30 provides the method of Embodiment 29, wherein the disease or disorder associated with oxidative, nitrosative, or nitrative stress is characterized by an elevated concentration of at least one redox biomarker in at least one bodily fluid.

Embodiment 31 provides the method of Embodiment 30, wherein the redox biomarker is at least one selected from the group consisting of peroxynitrite (ONOO⁻), hydrogen sulfide (H₂S), hydrogen peroxide (H₂O₂), superoxide (O₂·⁻), and nitric oxide (NO·).

Embodiment 32 provides the method of any one of Embodiments 29-31, wherein the contacting of the compound any of Formulae (I)—(IV) and the sample obtained from the subject results in chemical modification of the compound of any of Formulae (I)—(IV).

Embodiment 33 provides the method of any one of Embodiments 29-32, wherein the disease or disorder is at least one selected from the group consisting of cancer, diabetes, chronic inflammatory disease or disorder, pulmonary disease or disorder, cardiovascular disease or disorder, neurodegenerative disease or disorder, liver disease or disorder, kidney disease or disorder, and metabolic disease or disorder.

Embodiment 34 provides the method of any one of Embodiments 29-33, wherein the method further comprises administering to the subject at least one compound suitable to treat the disease or disorder associated with oxidative, nitrosative, or nitrative stress.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

What is claimed is:

1. A compound selected from the group consisting of: 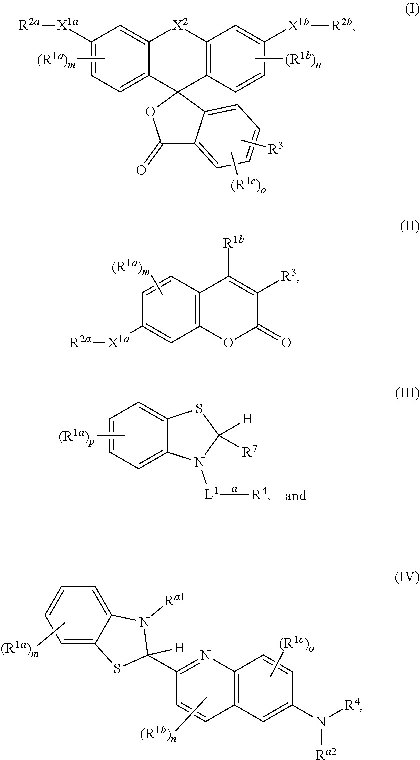
wherein:
R^{1a}, R^{1b}, R^{1c}, R^{1d}, R^{1e}, R^{1f}, and R^{1g}, if present, are each independently selected from the group consisting of optionally substituted C₁-C₆ alkyl, optionally substituted C₃-C₈ cycloalkyl, optionally substituted C₂-C₈ heterocycloalkyl, optionally substituted C₆-C₁₀ aryl, optionally substituted C₂-C₁₀ heteroaryl, halogen, CN, NO₂, OR^{A}, N(R^{A})(R^{B}), C(═O)OR^{A}, C(═O)N(R^{A})(R^{B}), S(═O)₂N(R^{A})(R^{B}), S(═O)N(R^{A})(R^{B}), OC(═O)R^{A}, and N(R^{A})C(═O)R^{B};
R^{2a} and R^{2b}, if present, are each independently selected from the group consisting of: 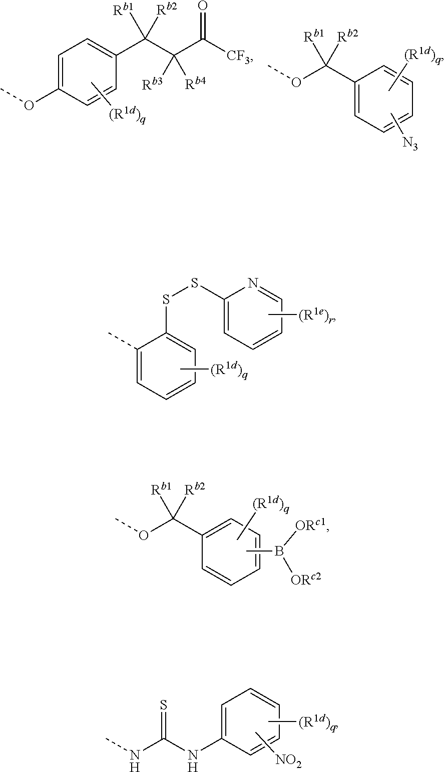
B(OR^{c1})(OR^{c2}), N(R^{A})(R^{B}), and OR^{A},
wherein no more than one of R^{2a} and R^{2b} is N(R^{A})(R^{B}) or OR^{A};
R³, if present, is 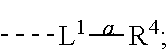
R⁴ is selected from the group consisting of optionally substituted C₁-C₃₀ alkyl, optionally substituted C₂-C₃₀ heteroalkyl, optionally substituted C₂-C₃₀ alkenyl, optionally substituted C₂-C₃₀alkynyl, 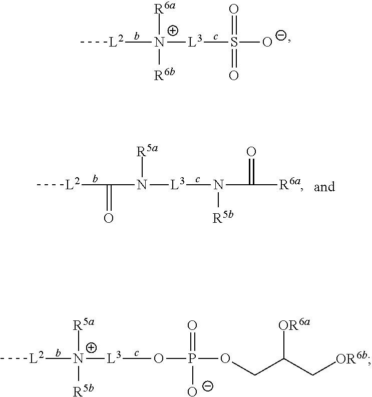
R^{5a} and R^{5b}, if present, are each independently selected from the group consisting of H, optionally substituted C₁-C₆ alkyl, optionally substituted C₃-C₈ cycloalkyl, optionally substituted C₆-C₁₀ aryl, and optionally substituted C₂-C₁₀ heteroaryl;
R^{6a} and R^{6b}, if present, are each independently selected from the group consisting of optionally substituted C₁-C₃₀ alkyl, optionally substituted C₂-C₃₀ heteroalkyl, optionally substituted C₂-C₃₀ alkenyl, optionally substituted C₂-C₃₀ alkynyl, C(═O)(optionally substituted C₁-C₃₀ alkyl), C(═O)(optionally substituted C₂-C₃₀ heteroalkyl), C(═O)(optionally substituted C₂-C₃₀ alkenyl), and C(═O)(optionally substituted C₂-C₃₀ alkynyl);
R⁷, if present, is 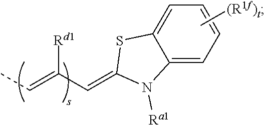
L¹, if present, is selected from the group consisting of: 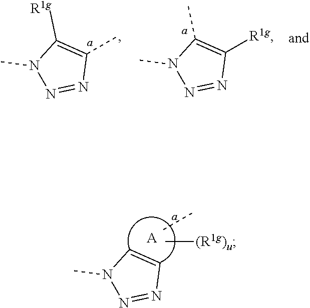
ring A, if present, is selected from the group consisting of C₅-C₁₂ cycloalkyl and C₂-C₁₀ heterocycloalkyl;
L² and L³ are each independently selected from the group consisting of optionally substituted C₁-C₁₂ alkylenyl, optionally substituted C₂-C₁₂ heteroalkylenyl, optionally substituted C₃-C₁₂ cycloalkylenyl, optionally substituted C₂-C₁₀ heterocycloalkylenyl, optionally substituted C₆-C₁₀ arylenyl, and optionally substituted C₂-C₁₂ heteroarylenyl;
X^{1a} and X^{1b}, if present, are each independently selected from the group consisting of a bond, —C(═O), —OC(═O)—, and —N(R^{A})C(═O)—;
X², if present, is selected from the group consisting of O and Si(R^{A})(R^{B});
R^{a1} and R^{a2} if present, are each independently selected from the group consisting of H, optionally substituted C₁-C₆ alkyl, C(═O)(optionally substituted C₁-C₆ alkyl), and hydrocarbyl;
R^{b1}, R^{b2}, R^{b3}, and R^{b4}, if present, are each independently selected from the group consisting of H and optionally substituted C₁-C₆ alkyl;
R^{c1} and R^{c2}, if present, are each independently selected from the group consisting of H and optionally substituted C₁-C₆ alkyl, or
R^{c1} and R^{c2} combine with the atoms to which they are bound to form an optionally substituted C₂-C₈ heterocycloalkyl;
each occurrence of R^{d1}, if present, is independently selected from the group consisting of H and optionally substituted C₁-C₆ alkyl,
wherein two R^{d1} groups having a 1,3-relationship can combine with the atoms to which they are bound to form an optionally substituted C₅-C₁₀ cycloalkenyl;
R^{A} and R^{B} are each independently selected from the group consisting of H, optionally substituted C₁-C₆ alkyl, optionally substituted C₃-C₈ cycloalkyl, optionally substituted C₂-C₈ heterocycloalkyl, optionally substituted C₂-C₆ alkenyl, optionally substituted C₂-C₆ alkynyl, optionally substituted C₆-C₁₀ aryl, optionally substituted C₂-C₁₀ heteroaryl;
m, n, and o are each independently selected from the group consisting of 0, 1, 2, and 3;
p, q, r, and t are each independently selected from the group consisting of 0, 1, 2, 3, and 4
s is selected from the group consisting of 1, 2, 3, and 4; and
u is selected from the group consisting of 0, 1, 2, 3, 4, 5, 6, 7, 8, 9, and 10;
or a salt, solvate, stereoisomer, or isotopologue thereof.

2. The compound of claim 1, wherein at least one of the following applies:
(a) R⁴ is selected from the group consisting of 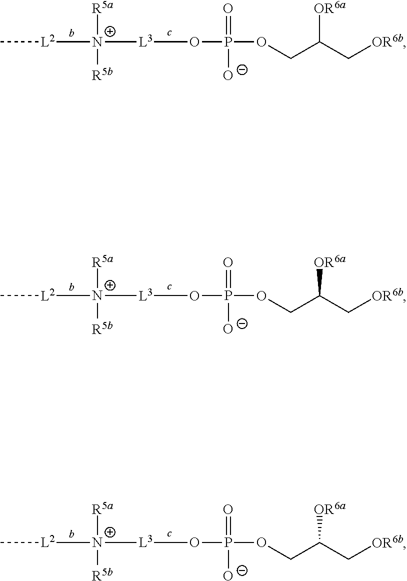
—(CH₂)₁₋₁₀C(═O)NH(CH₂)₂₋₁₀NHC(═O)(CH₂)₀₋₉CH₃, —(CH₂)₁₋₁₀N⁺[(CH₂)₀₋₉CH₃]₂(CH₂)₁₋₁₀S(═O)₂O⁻, and 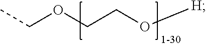
(b) L² and L³ are each independently selected from the group consisting of —CH₂—, —(CH₂)₂—, 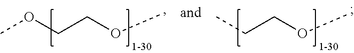
(c) R^{5a} and R^{5b} are each independently CH₃; and
(d) R^{6a} and R^{6b} are each independently C(═O)(C₁₀-C₂₀ alkyl), optionally wherein R^{6a} and R^{6b} are each independently C(═O)(pentadecanyl).

3. The compound of claim 1, wherein at least one of the following applies:
(a) R⁴ is: 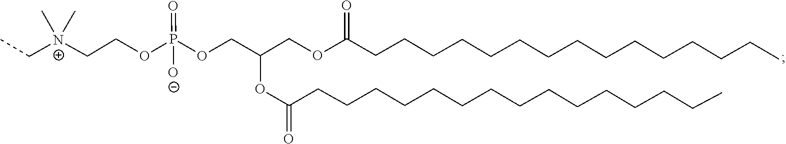
(b) LU is selected from the group consisting of: 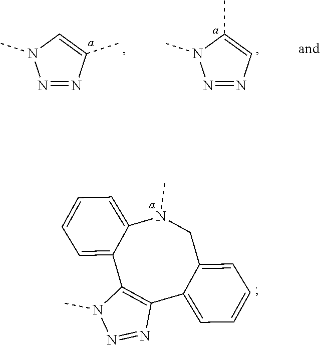
and
(c) at least one of R^{2a} and R^{2b} is selected from the group consisting of:
B(OH)₂,

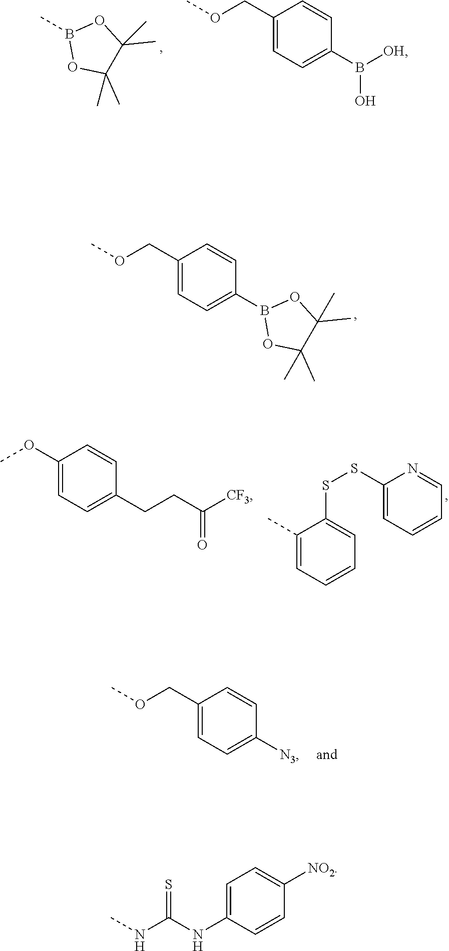

4. The compound of claim 1, wherein the compound is selected from the group consisting of:

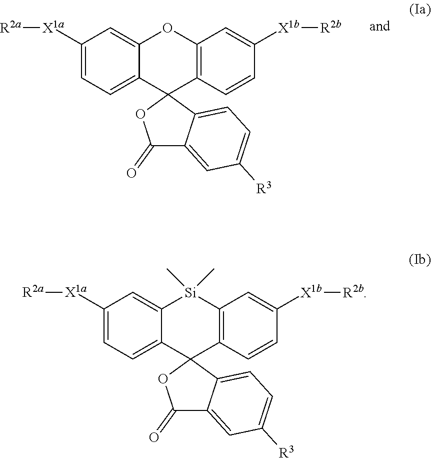

5. The compound of claim 4, wherein one of the following applies:
(a) X^{1a} and X^{2a} combine to form 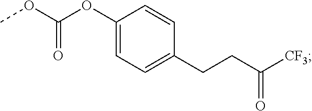
and X^{1b} and X^{2b} combine to form 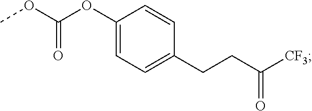
(b) X^{1a} and X^{2a} combine to form 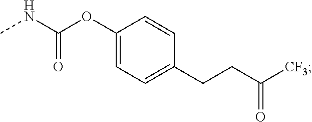
and X^{1b} and X^{2b} combine to form 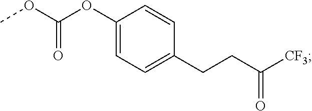
(c) X^{1a} and X^{2a} combine to form 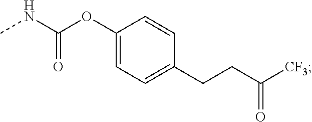
and X^{1b} and X^{2b} combine to form 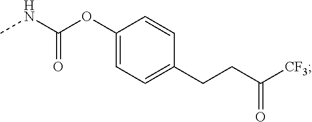
(d) X^{1a} and X^{2a} combine to form 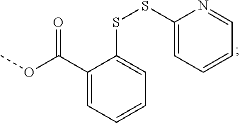
and X^{1b} and X^{2b} combine to form 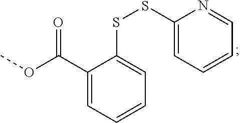
(e) X^{1a} and X^{2a} combine to form 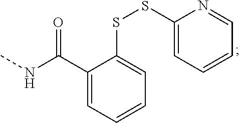
and X^{1b} and X^{2b} combine to form 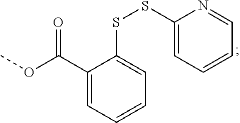
(f) X^{1a} and X^{2a} combine to form 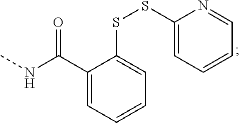
and X^{1b} and X^{2b} combine to form 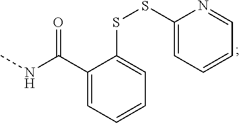
(g) X^{1a} and X^{2a} combine to form 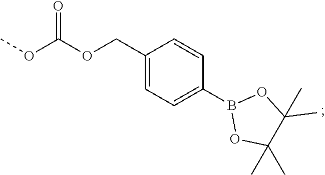
and X^{1b} and X^{2b} combine to form 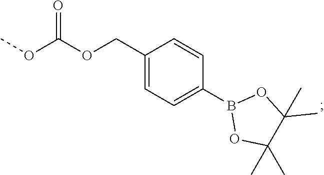
(h) X^{1a} and X^{2a} combine to form 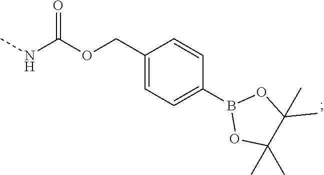
and X^{1b} and X^{2b} combine to form 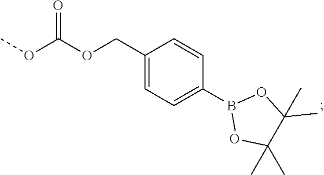
and
(i) X^{1a} and X^{2a} combine to form 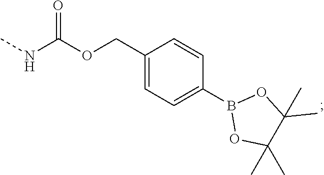
and X^{1b} and X^{2b} combine to form

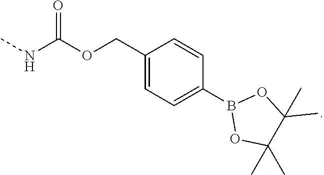

6. The compound of claim 1, wherein the compound is:

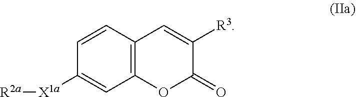

7. The compound of claim 6, wherein one of the following applies:
(a) X^{1a} and X^{2a} combine to form 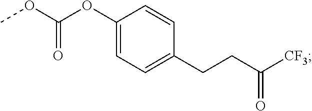
(b) X^{1a} and X^{2a} combine to form 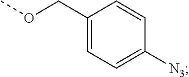
(c) X^{1a} and X^{2a} combine to form 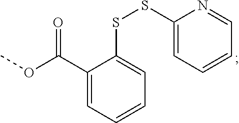
and
(d) X^{1a} and X^{2a} combine to form

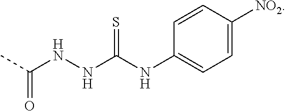

8. The compound of claim 1, wherein the compound is:

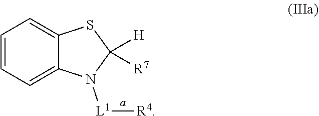

9. The compound of claim 8, wherein at least one of the following applies:
(a) L¹ is —(CH₂)₂—;
(b) R⁷ is selected from the group consisting of: 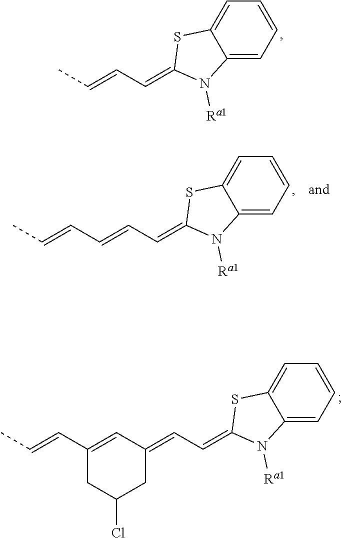
and
(c) R^{a1} is selected from the group consisting of H and CH₃.

10. The compound of claim 1, wherein the compound is:

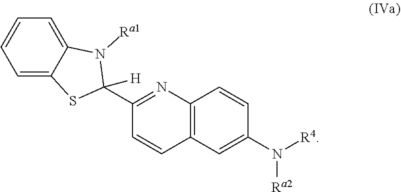

11. The compound of claim 10, wherein R^{a1} and R^{a2} are each independently selected from the group consisting of H and CH₃.

12. The compound of claim 1, which is selected from the group consisting of:

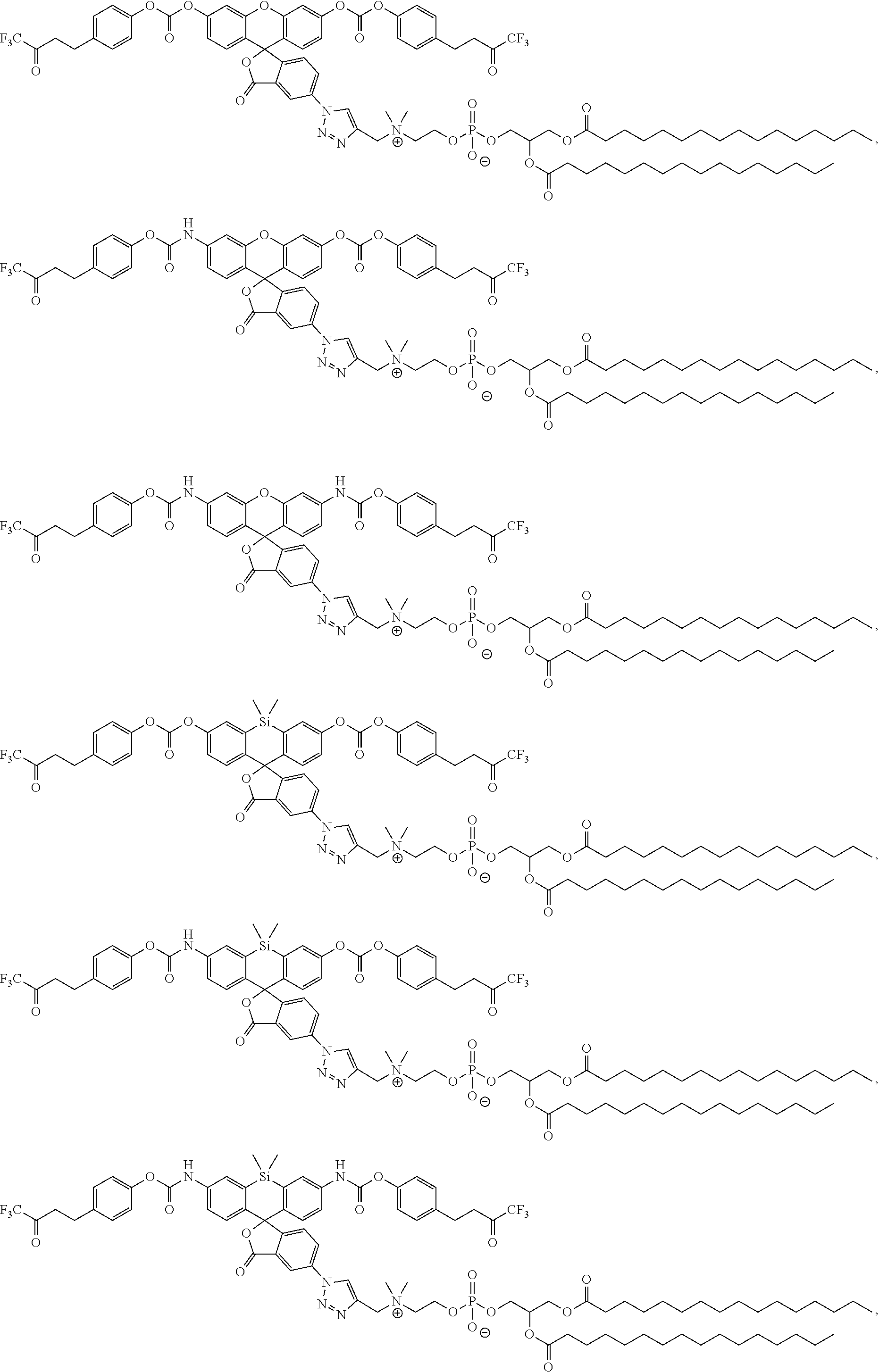

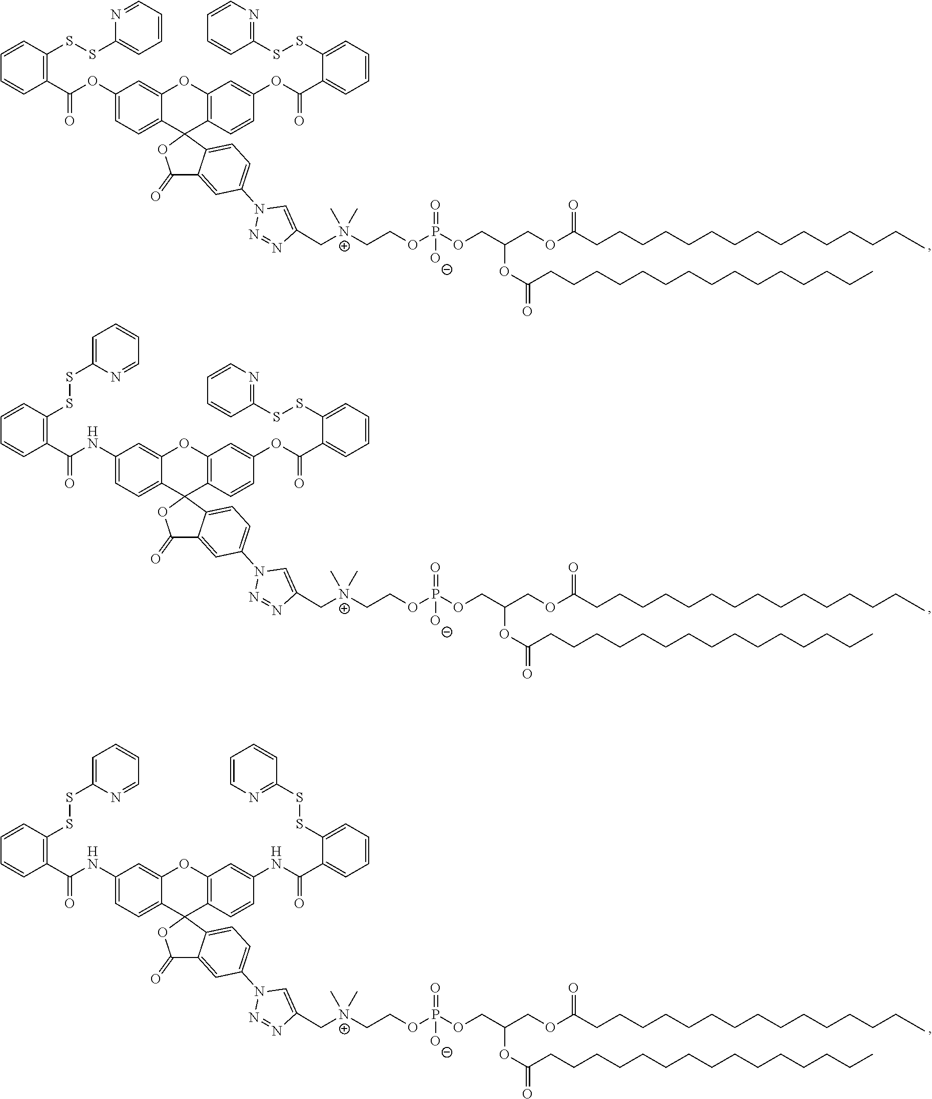

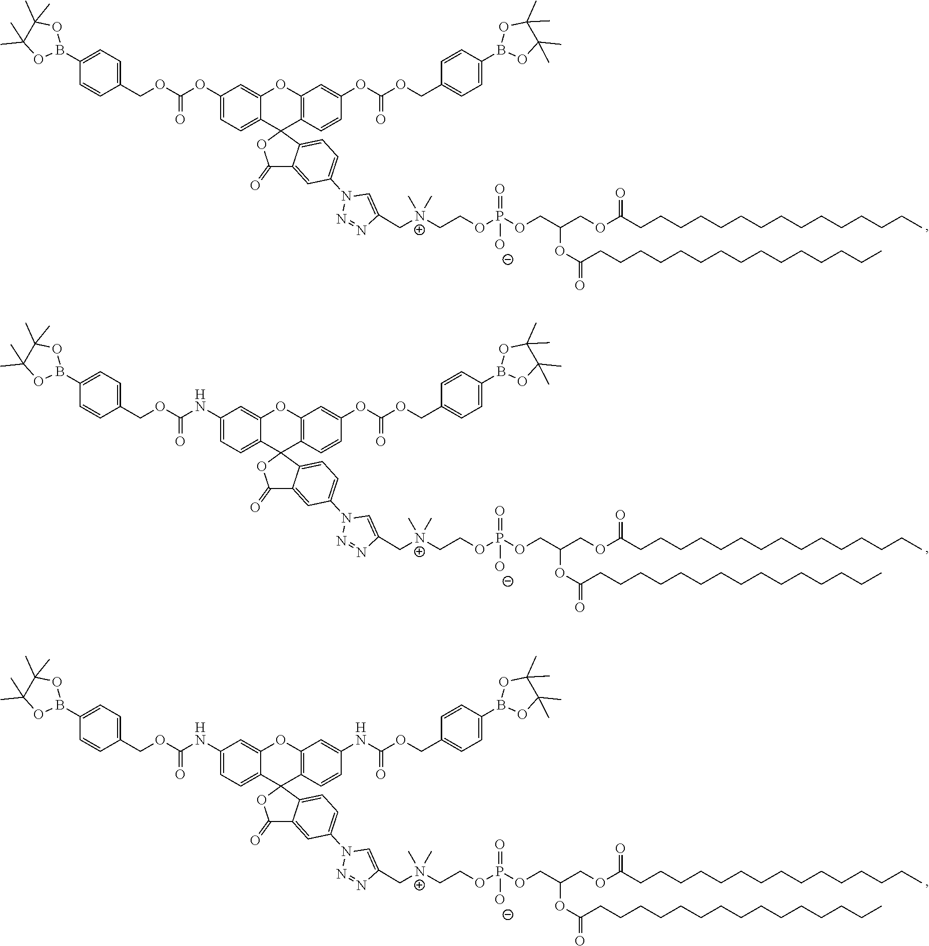

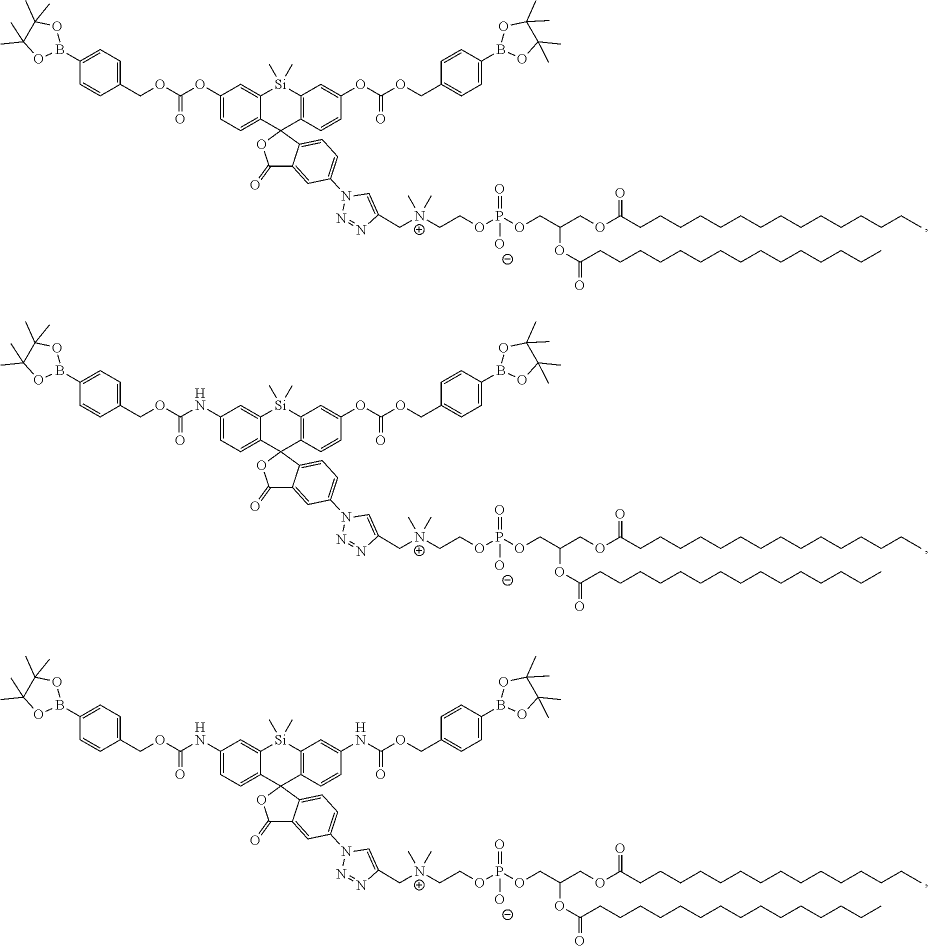

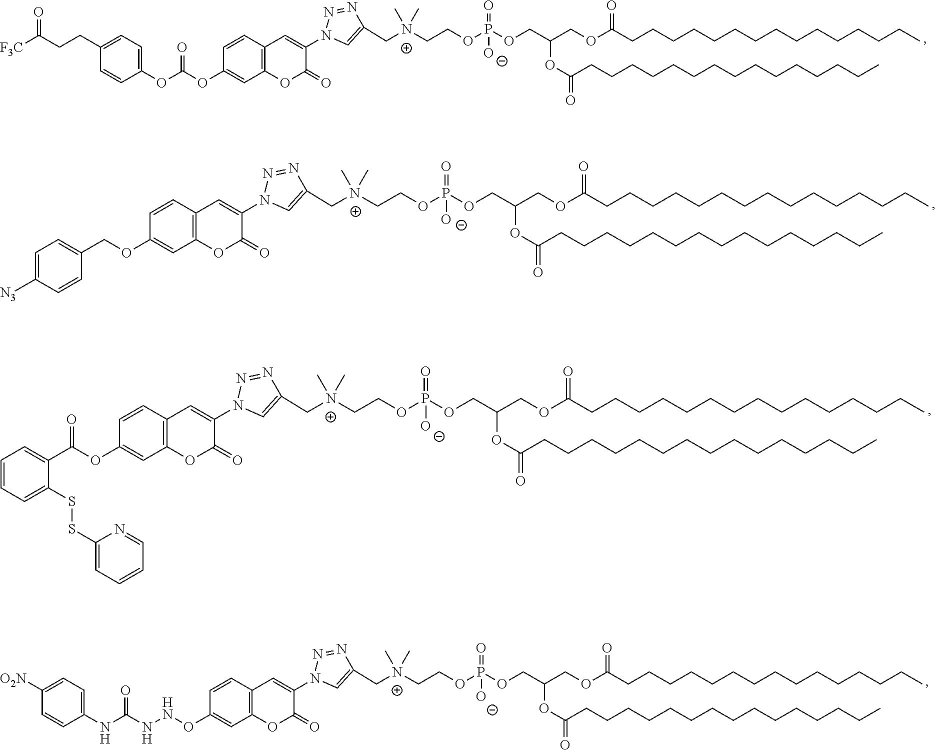

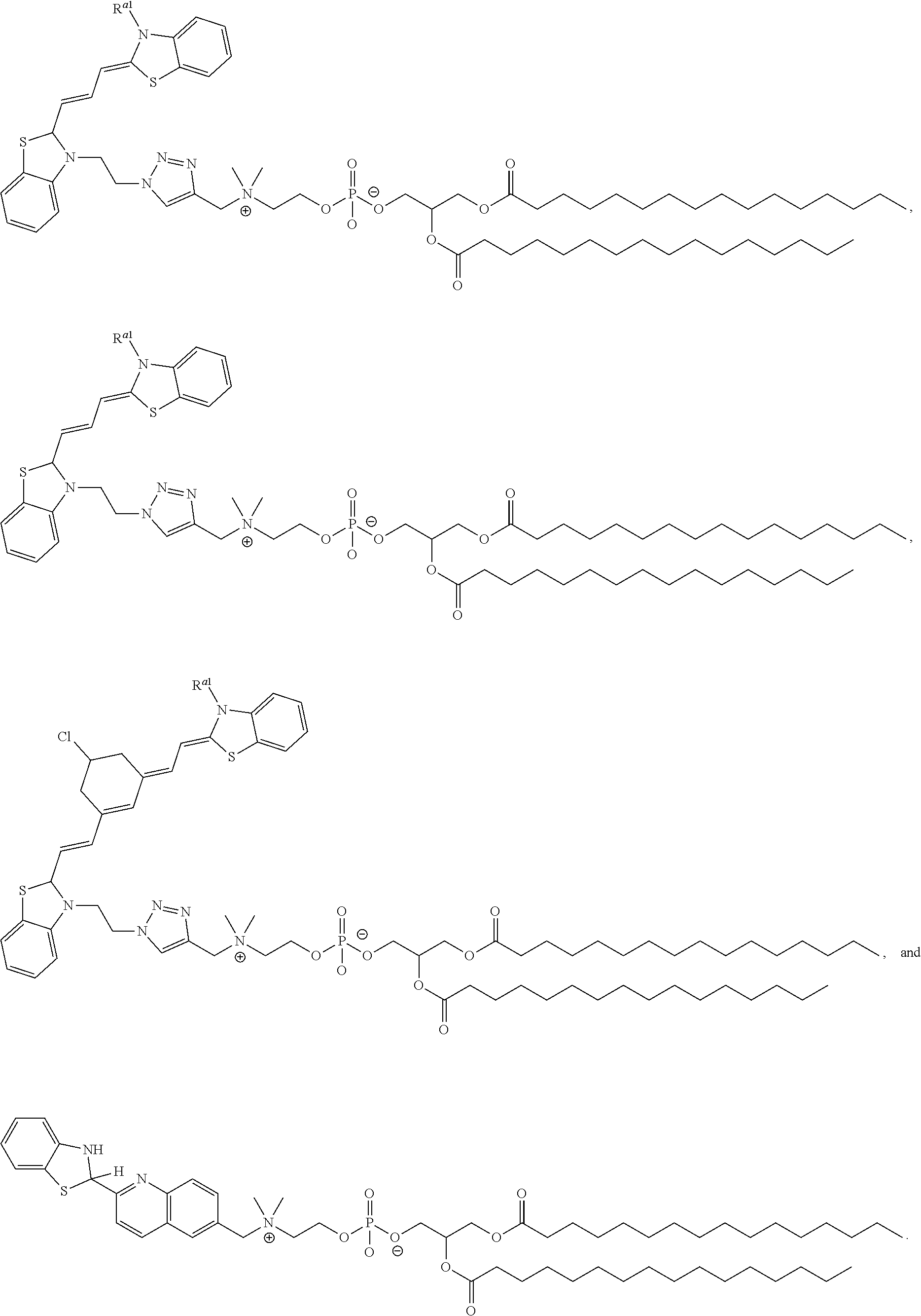

13. A lipid particle comprising:
(a) at least one compound of claim 1; and
(b) at least one additional lipid.

14. The lipid particle of claim 13, wherein at least one of the following applies:
(i) the at least one compound and the at least one additional lipid have a molar ratio ranging from about of about 1:99 to about 10:90 (compound:additional lipid);
(ii) the at least one additional lipid comprises at least one selected from the group consisting of a phospholipid and cholesterol, or a modified derivative thereof; and
(c) the at least one additional lipid comprises at least one selected from the group consisting of POPC, 1-hexadecanoyl-2-(9Z-octadecenoyl)-sn-glycero-3-phosphoglycerol (POPG), DOTMA, DOPE, and cholesterol.

15. A method of detecting a redox biomarker in a sample, the method comprising:
(a) contacting the compound of any of Formulae (I)—(IV) of claim 1 or a lipid particle thereof with the sample to provide a mixture; and
(b) detecting a fluorescent signal in the mixture, wherein the fluorescent signal is indicative of a redox biomarker in the sample.

16. The method of claim 15, wherein at least one of the following applies:
(a) the sample is a biological sample obtained from a subject;
(b) the redox biomarker is selected from the group consisting of peroxynitrite (ONOO⁻), hydrogen sulfide (H₂S), hydrogen peroxide (H₂O₂), superoxide (O₂·⁻), and nitric oxide (NO·); and
(c) the contacting of the compound any of Formulae (I)—(IV) with the redox biomarker in the sample results in chemical modification of the compound of any of Formulae (I)—(IV).

17. A method of diagnosing a disease or disorder associated with oxidative, nitrosative, or nitrative stress in a subject, the method comprising:
(a) contacting the compound of any of Formulae (I)—(IV) of claim 1 or a lipid particle thereof with a sample obtained from the subject to provide a mixture; and
(b) detecting a fluorescent signal in the mixture, wherein the fluorescent signal is indicative of a disease or disorder associated with oxidative, nitrosative, or nitrative stress.

18. The method of claim 17, wherein at least one of the following applies:
(a) the disease or disorder associated with oxidative, nitrosative, or nitrative stress is characterized by an elevated concentration of at least one redox biomarker in at least one bodily fluid;
(b) the redox biomarker is at least one selected from the group consisting of peroxynitrite (ONOO⁻), hydrogen sulfide (H₂S), hydrogen peroxide (H₂O₂), superoxide (O₂·⁻), and nitric oxide (NO·); and
(c) the contacting of the compound any of Formulae (I)—(IV) and the sample obtained from the subject results in chemical modification of the compound of any of Formulae (I)—(IV).

19. The method of claim 17, wherein the disease or disorder is at least one selected from the group consisting of cancer, diabetes, chronic inflammatory disease or disorder, pulmonary disease or disorder, cardiovascular disease or disorder, neurodegenerative disease or disorder, liver disease or disorder, kidney disease or disorder, and metabolic disease or disorder.

20. The method of claim 17, wherein the method further comprises administering to the subject at least one compound suitable to treat the disease or disorder associated with oxidative, nitrosative, or nitrative stress.
